(19) United States
(12) Patent Application Publication
(10) Pub. No.: US 2025/0325710 A1
(43) Pub. Date: Oct. 23, 2025

(54) Advanced In Vivo Platform to Study Human Neural Maturation and Circuit Integration

(71) Applicant: The Board of Trustees of the Leland Stanford Junior University, Stanford, CA (US)

(72) Inventors: Sergiu P. Pasca, Stanford, CA (US); Omer Revah, Stanford, CA (US); Felicity Gore, Stanford, CA (US); Kevin Kelley, Stanford, CA (US); Karl Deisseroth, Stanford, CA (US)

(21) Appl. No.: 18/870,244

(22) PCT Filed: Apr. 19, 2023
(86) PCT No.: PCT/US2023/019129

§ 371 Date: Nov. 27, 2024

Related U.S. Application Data
(60) Provisional application No. 63/350,367, filed on Jun. 8, 2022.
(60) Provisional application No. 63/351,147, filed on Jun. 10, 2022.

(51) Int. Cl. A61K 49/00

(52) U.S. Cl. CPC A61K 49/0008

(57) ABSTRACT

The present disclosure provides a method of producing a non-human mammalian animal model comprising human neural tissue, the method comprising introducing a first human neural organoid into a central nervous system location of a newborn non-human mammal; and allowing the newborn non-human mammal to mature to produce the non-human mammalian animal model comprising human neural tissue. The present disclosure provides a method of modeling a neuropsychiatric disorder. The present disclosure also provides a method of determining the effectiveness of a candidate agent on a neuropsychiatric disorder. The present disclosure provides a method for altering the behavior of a mammal. Also provided are non-human mammalian animal models comprising human neural tissue.

### CROSS-REFERENCE TO RELATED APPLICATIONS

Pursuant to 35 U.S.C. § 119 (e), this application claims priority to the filing dates of U.S. Provisional Application Ser. No. 63/350,367 filed on Jun. 8, 2022 and U.S. Provisional Application Ser. No. 63/351,147 filed on Jun. 10, 2022, the disclosures of which applications are herein incorporated by reference.

### ACKNOWLEDGEMENT OF GOVERNMENT RIGHTS

This invention was made with Government support under contract MH115012 awarded by the National Institutes of Health. The Government has certain rights in the invention.

### INTRODUCTION

Human brain development is a remarkable self-organizing process in which cells proliferate, differentiate, migrate, and wire to form functioning neural circuits that are subsequently refined by sensory experience (Kelley, K. W. et al. Cell. 2022 Jan. 6;185 (1): 42-61). A critical challenge to understanding brain development, particularly in the context of disease, is a lack of access to human brain tissue. By applying instructive signals to human induced pluripotent stem cells (hiPSC) grown in tridimensional (3D) cultures, the generation of self-organizing organoids resembling specific brain regions was previously shown, including that of regionalized neural organoids also known as human cortical spheroids (hCS) (Pasca, A. M. et al. Nat Methods. 2015 July; 12 (7): 671-8). hCS recapitulate certain features of the cerebral cortex (Pasca, A. M. et al. Nat Methods. 2015 July; 12 (7): 671-8; Yoon, S. J. et al. Nat Methods. 2019 January; 16 (1): 75-78), including specification of cortical progenitors, neurons and astrocytes, and they can be assembled with other organoids to study cell migration (Birey, F. et al. Nature. 2017 May 4; 545 (7652): 54-59); however, there are several limitations that restrict their broader applications in understanding neural circuit development and function. Specifically, in vitro systems lack the microenvironment that guide development in vivo. Moreover, hCS do not receive meaningful sensory input that shapes neural circuits. Finally, they are not integrated into circuits that can generate behavioral outputs, and this is critical in modeling behaviorally-defined neuropsychiatric disease.

### SUMMARY

Provided herein are methods for the production of non-human mammalian animal models comprising human neural tissue as the result of transplantation of human derived neural organoids. Also provided are methods for modeling human neuropsychiatric disorders in non-human mammalian animal models.

The present disclosure provides a method of producing a non-human mammalian animal model comprising human neural tissue, the method comprising introducing a first human neural organoid into a central nervous system location of a newborn non-human mammal; and allowing the newborn non-human mammal to mature to produce the non-human mammalian animal model comprising human neural tissue.

In some cases, the present disclosure provides a method of modeling a neuropsychiatric disorder, the method comprising introducing a first human neural organoid produced from a cellular biological sample from an individual living with a neuropsychiatric disorder into a first central nervous system location of a newborn non-human mammal; allowing the newborn non-human mammal to mature to produce the non-human mammalian animal model comprising a first human neural tissue; and characterizing the first human neural tissue to model the neuropsychiatric disorder.

In some cases, the present disclosure provides a method of determining the effectiveness of a candidate agent on a neuropsychiatric disorder, the method comprising administering the candidate agent to the non-human mammalian animal model produced by the methods of the invention; assaying the human neural tissue; and comparing the results of the assaying with mammals administered a control agent that is not the candidate agent.

In some cases, the present disclosure provides a method for altering the behavior of a mammal, the method comprising introducing a first human neural organoid into a first central nervous system location of a newborn non-human mammal; allowing the newborn non-human mammal to mature to produce a non-human mammal comprising a first human neural tissue; implanting an optical fiber into the first human neural tissue; training the mammal on an operant conditioning paradigm using the optical fiber; and stimulating the first human neural tissue using the optical fiber, wherein the first human neural tissue comprises a first light activatable polypeptide, wherein stimulating the first human neural tissue results in a change in the activity of the neurons in said first human neural tissue; to alter the behavior of the mammal.

These and other objects, advantages, and features of the invention will become apparent to those persons skilled in the art upon reading the details of the subject methods and compositions as more fully described below.

Non-human mammalian animal models are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-11. Transplantation of human cortical organoids in the developing rat cortex a. Schematic of experimental design. hCS generated from hiPSC are transplanted at day 30-60 of differentiation into the somatosensory cortex of newborn athymic rats b. Coronal and top view T2-weighted MR images showing t-hCS in somatosensory cortex at 2 months post-transplantation. c. Quantification of transplantations success rate shown per hiPSC line and cortical/subcortical position d. Coronal MRI images (left) and corresponding 3D volume reconstructions showing t-hCS growth over three months. e. Overview of example t-hCS in rat cortex. Scale bar, 1 mm. f. Representative immunocytochemistry images of t-hCS showing from top, left to right: PPP1R17 (4 months), CTIP2 (4 months) PDGRFα (8 months), NeuN (8 months), SOX9 and GFAP (8 months) and IBA1 (8 months). Scale bar, 20 μm. Co-expression of HNA indicates cells of human origin. g. Single nuclei RNA-seq: UMAP visualization of all clustered high-quality t-hCS nuclei after Seurat integration (n=3 t-hCS samples from 2 hiPSC lines). h. Gene expression violin plots for selected marker genes. i. GO term enrichment analysis (one-sided Fisher's exact test) of genes significantly up-regulated in t-hCS glutamatergic neurons compared to hCS glutamatergic neurons. Cyc. prog., cycling progenitor; OPC, oligodendrocyte progenitor cell, Oligo, oligodendrocyte; GluN_UL, upper layer glutamatergic neuron; GluN_DL, deep layer glutamatergic neuron; GluN_DL/SP, deep layer and subplate glutamatergic neurons; RELN, Reelin neurons.

FIG. 2A-2P. Advanced neuronal features in t-hCS reveal activity-dependent disease phenotypes in human cortical neurons a. 3D-reconstruction of biocytin-filled hCS and t-hCS neurons at 8 months of in vitro and, respectively, in vivo differentiation showing differences in dendrite complexity and morphology. b. Quantification of morphological features (soma diameter, total number of dendrites and total dendritic length) (hCS: n=8 neurons from 3 hiPSC lines, 1-2 hCS per line; t-hCS: n=6 neurons from 3 hiPSC lines, 1 animal per line; t-test, ** P<0.01, t-test, *P<0.05, Kolmogorov-Smirnov test, *** P<0.001, respectively). c. 3D-reconstructed dendritic branches of hCS and t-hCS at 8 months of differentiation. Red asterisks indicate putative dendritic spines. Quantification of dendritic spine density (hCS: n=8 neurons from 3 hiPSC lines, 1-2 hCS per line; t-hCS: n=6 neurons from 3 hiPSC lines, 1 animal per line; t-test, *** P<0.001. d. Quantification of the resting membrane potential (hCS: n=25 neurons from 3 hiPSC lines, 1-3 hCS per line; t-hCS: n=18 neurons from 3 hiPSC lines, 1-2 animals per line, Kolmogorov-Smirnov test, *** P<0.001). e. Repetitive action potential firing in hCS and t-hCS induced by increasing current injections and quantification of the maximal firing rate (hCS: n=25 neurons from 3 hiPSC lines, 1-3 hCS per line; t-hCS: n=18 neurons from 3 hiPSC lines, 1-2 animals per line, Kolmogorov-Smirnov test, *** P<0.001). f. Current traces showing spontaneous EPSCs in hCS and t-hCS neurons at 8 months of differentiation and quantification of synaptic events frequency (hCS: n=25 neurons from 3 hiPSC lines, 1-3 spheroid per lines; t-hCS: n=18 neurons from 3 hiPSC lines, 1-2 animals per line; Kolmogorov-Smirnov test, *** P<0.001). For FIGS. 2b-f, hCS and t-hCS from line 1208-2 are taken from the same differentiation batch maintained in parallel. g. Gene set enrichment analysis (one-sided Fisher's exact test) of genes significantly up-regulated in t-hCS glutamatergic neurons compared to hCS glutamatergic neurons with gene sets of both early-response (ERG) and late-response (LRG) activity-dependent genes identified from an in-vivo mouse study¹³ and human-specific LRGs from in vitro neurons¹⁴. h. Scaled and pseudobulked expression across snRNA-seq replicates of above LRG genes that are significantly up-regulated in t-hCS glutamatergic neurons. i. Immunostaining showing SCG2 expression in t-hCS (top) and hCS (bottom) neurons. Scale bar, 25 μm. j. Transplantation of hCS generated from 3 control and 3 TS hiPSC lines into newborn rats. k. 3D-reconstruction of biocytin-filled t-hCS neurons from TS and controls at 8 months of. l. Quantification of the mean dendrite length (control: n=19 neurons from 3 hiPSC lines, 1-2 spheroid per line; TS: n=21 neurons from 3 hiPSC lines, 1-2 animals per line. Kolmogorov-Smirnov test, *** P<0.001). m. 3D-reconstructed dendritic branches from hCS and t-hCS at 8 months of differentiation and quantification of dendritic spine density (Control: n=16 neurons from 3 hiPSC lines, 1-2 hCS per line; TS: n=21 neurons from 3 hiPSC lines, 1-2 animals per line. Kolmogorov-Smirnov test, *** P<0.001). Red asterisks indicate putative dendritic spines. n. Current traces showing sEPSCs in hCS and t-hCS neurons at 8 months of differentiation. o. Cumulative frequency plots and quantification of synaptic events frequency and amplitude (Control: n=32 neurons from 3 hiPSC lines, 1-2 hCS per line; TS: n=26 neurons from 3 hiPSC lines, 1-2 animals per line. Kolmogorov-Smirnov test, ** P<0.01). p. Sholl analysis comparison of the dendritic complexity of TS neurons and control neurons in hCS and t-hCS. Dotted line shows postnatal human L2/3 pyramidal neurons for comparison.

FIG. 3A-3S. Transplanted hCS receive sensory-related inputs a-c. Monosynaptic rabies tracing to identify anatomical inputs to t-hCS. a. Schematic of approach for identification of inputs to t-hCS using monosynaptic rabies tracing. b. Top, representative images of GFP and the human-specific marker STEM121 expression at the border between t-hCS and rat cortical cells. Bottom, GFP expression in the rat ipsilateral ventrobasal nucleus (VB, left) and ipsilateral somatosensory cortex (S1, right). Scale bars, 50 μm c. Quantification of GFP-expressing cells (n=4 rats). d-e. Identification of netrin-G1 positive thalamic terminals in t-hCS. d. Bright field image of coronal section containing t-hCS and VB. e. Representative images of netrin-G1 and STEM121 expression in t-hCS (left) and VB (right). f-h. Electrophysiological characterization of rat-human connectivity. f. Schematic of experimental preparation. g. Left, Current traces from a representative t-hCS neuron following electrical stimulation in nearby rat somatosensory cortex with (purple) or without (black) NBQX. Middle, quantification of EPSC amplitude with or without NBQX (paired t-test, *P<0.05). Right, percentage of t-hCS neurons that displayed EPSCs in response to electrical stimulation of rat somatosensory cortex. h. Left, Current traces from a representative t-hCS neuron following electrical stimulation in the rat internal capsule with (purple) or without (black) NBQX. Middle, quantification of EPSC amplitude with or without NBQX (Wilcoxon test, *P<0.05). Right, percentage of t-hCS neurons that displayed EPSCs in response to electrical stimulation of the rat internal capsule. i-j. Spontaneous activity in t-hCS. i. Left, schematic of experimental preparation for two-photon calcium imaging of spontaneous activity in t-hCS. Middle, GCaMP6 s expression in t-hCS at approximately 150 days post-transplantation. Right, example timelapse images of GCaMP6 s fluorescence. j. Top, heatmap of z-scored fluorescence traces from anesthetized recording of spontaneous activity. Bottom, population-averaged z-scored activity. k-s. Evoked activity in t-hCS. k. Schematic of experimental preparation for two-photon calcium imaging of t-hCS activity in response to whisker deflection. l. Single trial responses to whisker stimulation from a representative example cell. Top, heatmap of single trial z-scored fluorescence traces aligned to whisker deflection at time zero (left) or randomly generated timestamps (right). Bottom, trial-averaged z-scored activity. m. Population-averaged z-scored responses of all cells aligned to whisker deflection at time zero (red) or randomly generated timestamps (grey). n. Schematic of experimental preparation for extracellular electrophysiological recording of optogenetically identified t-hCS neurons in response to whisker deflection. o. Representative raw voltage traces from a putative t-hCS unit during blue laser stimulation (left) or whisker deflection (right). Red arrows indicate the first light-evoked (left) or whisker deflection-evoked (right) spike. p. Spike waveforms of light and whisker deflection responses from the putative unit shown in panel o. q. Single trial responses to whisker stimulation from a representative light-responsive example single unit. Top, raster plot of single trial spiking activity aligned to whisker deflection at time zero (left) or randomly generated timestamps (right). Bottom, trial-averaged z-scored firing rate. r. Population-averaged z-scored firing rates of all light-responsive units aligned to whisker deflection at time zero (red) or randomly generated timestamps (grey). s. Quantification of evoked spiking activity. Left, proportion of light-responsive units significantly modulated by whisker deflection (n=3 rats). Right, latency to peak z-score (n=3 rats, n=5 (light green), 4 (dark green), and 4 (cyan) whisker deflection-modulated units per rat).

FIG. 4A-4M. Transplanted hCS make functional connections onto rat neurons and modulate behavior a. t-hCS axonal projections throughout the rat brain. Top, schematic of approach for identifying t-hCS axonal projections. Bottom, Representative images of EYFP expression in t-hCS axons throughout the rat brain. Scale bars, 100 μm. b-g. Electrophysiological characterization of human-rat connectivity. b. Schematic of experimental approach. c. Example blue light-evoked photocurrents (top) and voltage responses (bottom) in EYFP⁺ t-hCS cells. d. Example blue light-evoked photocurrents (top) and voltage responses (bottom) in EYFP⁻ rat cells. e. Current traces from an example rat neuron following blue light stimulation of t-hCS axons with TTX and 4-AP (green), TTX (grey), or in aCSF (black). f. Current traces from example rat neuron following blue light stimulation of t-hCS axons with (purple) or without (black) NBQX. g. Left, latency of blue light-evoked responses in rat cells (n=16 cells, latency=7.13+/−0.5 ms). Middle, amplitude of light evoked EPSCs recorded with or without NBQX (n=7 cells, paired t-test, *** P<0.001). Right, percentage of rat cells that demonstrated EPSCs in response to blue light. h-l. Behavioral characterization of human-rat connectivity. h. Schematic of behavioral task. i. Behavioral performance of an example animal on day 1 (left) or day 15 (right) of training. Left, mean number of licks performed on all blue or red light trials on day 1 (n=150 blue light trials, n=150 red light trials). Left center, cumulative lick count across red and blue light trials on day 1. Right center, mean number of licks performed on all blue or red light trials on day 15 (n=150 blue light trials, n=150 red light trials, two-way ANOVA F(1, 586)=273.2, *** P<0.001). Right, cumulative lick count on red and blue light trials on day 15. j. Behavioral performance of all animals transplanted with t-hCS expressing ChR2-EYFP on day 1 or day 15 of training (n=9 rats, two-way repeated-measures ANOVA F(1, 8)=11.90, ** P<0.01). k. Behavioral performance of all animals transplanted with hCS expressing a control fluorophore on day 1 or day 15 of training (n=9, two-way repeated-measures ANOVA F(1, 8)=3.43, P>0.05). l. Evolution of preference score for all animals transplanted with hCS expressing either ChR2-EYFP or a control fluorophore (n=9 ChR2, n=9 control, two-way ANOVA F(1, 16)=18.93, *** P<0.001). m. c-Fos expression in response to optogenetic activation of t-hCS in somatosensory cortex. Left, example confocal images. Right, quantification of c-Fos expression across brain areas (n=3 stimulated ChR2 rats, n=3 unstimulated ChR2 rats, n=3 stimulated EYFP rats). Scale bar, 100 μm. Abbreviations: mPFC=medial prefrontal cortex, A1=auditory cortex, ACC=anterior cingulate cortex, piri=piriform cortex, d. striatum=dorsal striatum, l. septum=lateral septum, v. striatum=ventral striatum, S1=somatosensory cortex, HPC=hippocampus, VPM=ventral posteromedial nucleus of thalamus, MDT=mediodorsal nucleus of the thalamus, BLA=basolateral amygdala. VTA=ventral tegmental area, PAG=periaqueductal gray. * P<0.05, ** P<0.01, *** P<0.001.

FIG. 5A-5M. Schematic of the transplantation procedure and effects on animal behavior a-c. Schematics of the surgical approach. d. Left, t-hCS visualized with T2-weighted MRI. Right, t-hCS visualized with DAPI. e. Reconstructed volumes of the same t-hCS calculated from MRI or histological slices t-hCS in 3 brains. f. Quantification of MRI volume reconstruction over time (n=6 t-hCS from 3 hiPSC lines, 2 animals per hiPSC line; one-way repeated measure ANOVA, *P<0.05). g. Quantification of survival in transplanted animals over time. h-j. Animals transplanted with t-hCS do not show behavioral deficits or seizures. h. Distance traveled in open field arena by transplanted (orange) or non-transplanted control animals (grey) for each minute of a 10-minute testing session (left) and across the entire 10-minute testing period (right) (n=11 non-transplanted control rats, 9 transplanted rats; two-way repeated-measures ANOVA F(9, 162)=0.64, P>0.05). i. Discrimination index ((time spent interacting with novel object-time spent interacting with familiar object)/(time spent interacting with novel object+time spent interacting with familiar object)) during novel object test calculated for transplanted (orange) or non-transplanted control animals (grey) (n=11 non-transplanted control rats, 9 transplanted rats; t-test P>0.05). j. Freezing behavior during fear conditioning training, contextual fear memory test, and cued fear memory test for transplanted (orange) or non-transplanted control animals (grey) (n=11 non-transplanted control rats, 9 transplanted rats; two-way repeated-measures ANOVA F(2, 36)=1.32, P>0.05). k. Representative voltage traces from EEG recordings in the frontal and somatosensory cortices of non-transplanted (left) and transplanted (right) rats. l. Power spectral density plots of EEG activity recorded in the somatosensory cortex of non-transplanted control and transplanted rats (n=3 transplanted, 3 control rats; mixed model ANOVA F( )═, P>0.05). m. Power spectral density plots of somatosensory cortex EEG activity recorded simultaneously in the non-transplanted and transplanted hemisphere of individual rats (n=3 transplanted rats; mixed model ANOVA F( )═, P>0.05).

FIG. 6A-6E. Immunohistochemical characterization of t-hCS. a-b. NeuN expression in t-hCS and the surrounding rat brain. c. Quantification of the overlap of HNA and NeuN expression in t-hCS and rat cortex (n=5 t-hCS from 4 hiPSC lines, 1-2 t-hCS per line). d. Representative images of GAD65/67 expression in t-hCS-rat cortex border. e. Rarely observed HNA⁺ GAD65/67+neuron in t-hCS

FIG. 7A-7E. Immunohistochemical characterization of t-hCS continued a. Representative images of SATB2 and CTIP2 expression in t-hCS. b. Example images of SOX2 and NeuN expression in t-hCS. c. Representative images of rat-edothelial-marker-1 (RECA1) and IBA1 expression in t-hCS and nearby rat cortex 3 months after transplantation. d. HNA and IBA1 expression in t-hCS reveals microglia originate from rat. e. GFAP and IBA1 colocalization in t-hCS.

FIG. 8A-8H. Data quality of single nucleus RNA-seq samples and hCS analysis a. The number of snRNA-seq read counts aligned to rat and human genome for each nucleus split by sample. Human nuclei were defined as nuclei with >95% of total reads aligning to the human genome. b. snRNA-seq quality metrics showing the distribution of the number of counts, number of genes, and mitochondrial (MT) gene fraction per cell in each sample. MT gene fraction plotted as boxplots with outlier points shown (outside 1.5 times the interquartile range). Lines denote nuclei quality thresholds. c. Same integrated UMAP as shown in FIG. 1g, colored by t-hCS sample. d. Cell type proportions across t-hCS samples colored by clusters. e. UMAP dimensional reduction visualization of all clustered high-quality hCS nuclei after Seurat integration (n=3 t-hCS samples from 3 hiPSC lines). f. Gene expression violin plots for selected marker genes. g. Same integrated UMAP as shown in panel e, colored by hCS sample. h. Cell type proportions across hCS samples colored by clusters. hCS and t-hCS from 2242-1-d227 are taken from the same differentiation batch maintained in parallel. Cyc. prog., cycling progenitor; Astroglia, astrocyte lineage cell; IPC, intermediate progenitor cell; GluN_UL, upper layer glutamatergic neuron; GluN_DL, deep layer glutamatergic neuron; GluN_DL/SP, deep layer and subplate glutamatergic neurons; RELN, Reelin neurons; IN, GABAergic neurons; Choroid, choroid plexus-like cells; Mening., meningeal-like cells.

FIG. 9A-9J. RNA-seq integration with primary fetal and adult human cortical cell types Describes gene expression in primary fetal and adult human cortical cell types.

FIG. 10A-10C. Electrophysiological and morphological properties of glutamatergic hCS neurons a. Example 3D-reconstruction of biocytin-filled CamKII: eYFP-expressing hCS neuron at 8 months. b. Morphological properties of 3D reconstructed CamkII⁺ hCS neurons (n=8 neurons from 2 hiPSC lines, 1-2 hCS per line). c. Electrophysiological properties of CamKII⁺ hCS neurons (n=16 neurons from 2 hiPSC lines, 1-2 hCS per line).

FIG. 11A-11E Electrophysiological and morphological properties of cortical neurons from postnatal human cerebral cortex a. Schematics showing the location of resected specimens and recording conditions. b. 3D-reconstruction of biocytin-filled human L2/3 pyramidal neurons. c. Quantification of the soma diameter, number of primary branches, total number of dendrites, total length and spine density (n=12 L2/3 neurons from 2 specimens). d. Sholl analysis comparison of the dendritic complexity of L2/3 neurons (n=7 L2/3 neurons from sample 1 and n=8 neurons from sample 2). e. Quantification of membrane capacitance, resting membrane potential, maximal firing rate, spike amplitude, spike half-width and spike threshold of L2/3 pyramidal neurons (n=22 L2/3 neurons from 2 specimens).

FIG. 12A-12G Morphological and electrophysiological properties of control and TS t-hCS neurons a-c. Morphological properties of control and TS t-hCS neurons. a-b. Examples of 3D-reconstructed t-hCS neurons derived from control (3 hiPSC lines from 3 individuals pooled from 2 differentiations) and TS (3 hiPSC lines from 3 individuals, pooled from 2 differentiations). Line identities of filled cells from left to right, Control: 1208-2; 2242-1; 8119-1; 2242-1; 2242-1; 1208-2; and TS: 8303-S3; 8303-S3; 7643-6; 7643-6; 9862-2; 8303-S3; 8303-S3; 8303-S3; 8303-S3; 9862-2; 8303-S3; 7643-6. c. Quantification of the soma diameter, number of primary branches, total number of dendrites and total length (control: n=19 neurons from 3 hiPS cell lines, 1-2 animals per line; TS: n=21 neurons from 3 hiPSC lines, 1-2 animals per line; t-test, P=0.57, Kolmogorov-Smirnov test, *P<0.05, *P<0.05). d-g. Electrophysiological properties of control and TS t-hCS neurons. d. Comparison of the resting membrane potential in hCS and t-hCS (hCS: n=25 neurons from 3 hiPSC lines, 1-3 hCS per line; t-hCS: n=18 neurons from 3 hiPSC lines, 1-2 animals per line; t-test, *** P<0.001). e. Example traces of a single AP firing in control and TS t-hCS neurons, showing differences in AP height (black arrows) and threshold (gray dashed line). f. Comparison of electrophysiological features in control and TS t-hCS neurons, membrane capacitance, resting membrane potential, maximal firing rate, spike amplitude, spike half-width and spike threshold (Control: n=29-31 neurons from 3 hiPSC lines, 1-2 animals per line; TS: n=33-36 neurons from 3 hiPSC lines, 1-2 animals per line; resting membrane potential, *** P<0.001, max firing rate, *** P<0.001; spike amplitude, *** P<0.001; spike threshold, *** P<0.001). g. Cumulative frequency plots and quantification of spontaneous EPSCs decay time and charge (Control: n=32 neurons from 3 hiPSC lines, 1-2 hCS per line; TS: n=26 neurons from 3 hiPSC lines, 1-2 animals per line).

FIG. 13A-13G Electrophysiological characterization of inputs onto t-hCS a. Schematic of experimental preparation for electrically activating rat tissue while performing whole cell recordings from t-hCS neurons. b. Left, current traces from a representative t-hCS neuron following electrical stimulation in white matter with (purple) or without (black) NBQX. Middle, quantification of EPSC amplitude with or without NBQX (paired t-test, ** P<0.01). Right, percentage of t-hCS neurons that displayed EPSCs in response to electrical stimulation in white matter. c. Left, Current traces from a representative t-hCS neuron following electrical stimulation of t-hCS with (purple) or without (black) NBQX. Middle, quantification of EPSC amplitude with or without NBQX (Wilcoxon test, ** P<0.01). Right, percentage of t-hCS neurons that displayed EPSCs in response to electrical stimulation of t-hCS. d. Latency to EPSC in t-hCS neurons following electrical stimulation of somatosensory cortex (S1), internal capsule (IC), white matter (WM), or t-hCS. e. Schematic of experimental preparation for optogenetically activating rat thalamic terminals in t-hCS while performing whole cell recordings from t-hCS neurons. f. Top, example recorded t-hCS neuron. Bottom, current traces from a representative t-hCS neuron following optogenetic activation of rat thalamic terminals in t-hCS with (purple) or without (black) NBQX. g. Left, latency to EPSC in t-hCS neurons following optogenetic activation of rat thalamic terminals in t-hCS. Right, percentage of t-hCS neurons that displayed EPSCs in response to optogenetic activation of rat thalamic terminals in t-hCS.

FIG. 14A-14Q Characterization of t-hCS activity in vivo a-g. Characterization of spontaneous activity in t-hCS in vivo. a-c. Fiber photometry recordings of spontaneous t-hCS activity in vivo. a. Top, Schematic of experimental preparation for fiber photometry recording of spontaneous activity in t-hCS. Bottom, representative image of GcaMP6 s expression in t-hCS. Scale bar, 100 μm. b. Example z-scored fluorescence traces from awake recording of spontaneous activity. c-f. Extracellular recordings of spontaneous t-hCS activity in vivo. c. Top, Schematic of experimental preparation for extracellular electrophysiological recordings of spontaneous activity in t-hCS. Bottom, average waveforms of putative t-hCS units. d. Hidden Markov model used to identify ‘on’ and ‘off’ periods of population activity. Top, raster plot of spontaneous activity of simultaneously recorded units. Bottom, population averaged z-scored activity with ‘on’ states identified by Hidden Markov model overlaid in red. e. Quantification of spontaneous spiking activity. Left, number of spikes each unit contributed to each burst (n=4 rats, 10 (dark green), 12 (light green), 19 (cyan), and 14 (blue) units per rat). Left center, proportion of recorded units that were engaged in each burst (n=4 rats). Right center, ON period duration (n=4 rats). Right, OFF period duration (n=4 rats). f. Histogram of median correlations of each unit with all other simultaneously recorded units recorded in transplanted rats (putative human units, blue, n=48 units from 4 rats) or non-transplanted rats (rat units, red, n=56 units from 3 rats. t-test, *** P<0.001). g. Peak power spectral density frequency of spontaneous activity across all recording modalities used. h-q. Characterization of evoked activity in t-hCS in vivo. h-j. Fiber photometry recordings of t-hCS activity in response to whisker deflection. h. Schematic of experimental preparation for fiber photometry recording of t-hCS neurons in response to whisker deflection. i. Z-scored responses to whisker deflection at time zero (red) or randomly generated timestamps (grey) averaged across animals (n=3 rats). j. Quantification of mean z-score following whisker stimulation compared to baseline (*P<0.05). k-n. Extracellular electrophysiological recordings of t-hCS activity in response to whisker deflection. k. Schematic of experimental preparation for extracellular electrophysiological recordings of t-hCS activity in response to whisker deflection. l. Single trial responses to whisker stimulation from a representative example single unit. Top, raster plot of single trial spiking activity aligned to whisker deflection. Bottom, trial-averaged z-scored firing rate. m. Population-averaged z-scored firing rates of all cells aligned to whisker deflection at time zero (red) or randomly generated timestamps (grey). n. Quantification of evoked spiking activity. Left, proportion of units significantly modulated by whisker deflection (n=4 rats). Right, latency to peak z-score (n=4 rats, n=8 (dark green), 11 (light green), 12 (cyan), and 11 (blue) whisker deflection-modulated units per rat). o-q. Heatmaps of trial-averaged responses of all identified putative t-hCS cells across different recording modalities. o. Top, heatmap of trial-averaged z-scored fluorescence traces from all t-hCS cells recorded with two-photon calcium imaging aligned to whisker deflection (left) or randomly generated timestamps (right). Bottom, population-averaged z-scored fluorescence traces (n=14 cells from 1 rat). p. Top, heatmap of trial-averaged z-scored firing rates from all t-hCS units recorded with extracellular electrophysiology aligned to whisker deflection (left) or randomly generated timestamps (right). Bottom, population-averaged z-scored firing rates (n=42 units from 4 rats). q. Top, heatmap of trial-averaged z-scored firing rates from all opto-tagged t-hCS units recorded with extracellular electrophysiology aligned to whisker deflection (left) or randomly generated timestamps (right). Bottom, population-averaged z-scored firing rates (n=31 units from 3 rats).

The invention is best understood from the following detailed description when read in conjunction with the accompanying drawings.

### Definitions

By “pluripotency” and pluripotent stem cells it is meant that such cells have the ability to differentiate into all types of cells in an organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells, are derived from differentiated somatic cells, that is, cells that had a narrower, more defined potential and that in the absence of experimental manipulation could not give rise to all types of cells in the organism. hiPSC have a human ES-like morphology, growing as flat colonies with large nucleo-cytoplasmic ratios, defined borders and prominent nuclei. In addition, hiPSC express several pluripotency markers known by one of ordinary skill in the art, including but not limited to alkaline phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the hiPSC are capable of forming teratomas. In addition, they are capable of forming or contributing to ectoderm, mesoderm, or endoderm tissues in a living organism.

As used herein, “reprogramming factors” refers to one or more, i.e. a cocktail, of biologically active factors that act on a cell to alter transcription, thereby reprogramming a cell to multipotency or to pluripotency. Reprogramming factors may be provided to the cells individually or as a single composition, that is, as a premixed composition, of reprogramming factors. The factors may be provided at the same molar ratio or at different molar ratios. The factors may be provided once or multiple times in the course of culturing the cells of the subject invention. In some embodiments the reprogramming factor is a transcription factor, including without limitation, Oct3/4; Sox2; Klf4; c-Myc; Nanog; and Lin-28.

Somatic cells are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector. The somatic cells may be fibroblasts, adipocytes, stromal cells, and the like, as known in the art. Somatic cells or hiPSC can be obtained from cell banks, from normal donors, from individuals having a neurologic or psychiatric disease of interest, etc.

Following induction of pluripotency, hiPSC are cultured according to any convenient method, e.g., on irradiated feeder cells and commercially available medium. The hiPSC can be dissociated from feeders by digesting with protease, e.g., dispase, preferably at a concentration and for a period of time sufficient to detach intact colonies of pluripotent stem cells from the layer of feeders. The organoids can also be generated from hiPSC grown in feeder-free conditions, by dissociation into a single cell suspension and aggregation using various approaches, including centrifugation in plates, etc.

Genes may be introduced into the somatic cells or the hiPSC derived therefrom for a variety of purposes, e.g., to replace genes having a loss of function mutation, provide marker genes, etc. Alternatively, vectors are introduced that express antisense mRNA, siRNA, ribozymes, etc. thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as BCL-2. Various techniques known in the art may be used to introduce nucleic acids into the target cells, e.g., electroporation, calcium precipitated DNA, fusion, transfection, lipofection, infection and the like, as discussed above. The particular manner in which the DNA is introduced is not critical to the practice of the invention.

Disease-associated or disease-causing genotypes can be generated in healthy hiPSC through targeted genetic manipulation (CRISPR/CAS9, etc.) or hiPSC can be derived from individuals that carry a disease-related genotype or are diagnosed with a disease. Moreover, neural and neuromuscular diseases with less defined or without genetic components can be studied within the model system. A particular advantage of this method is the fact that edited hiPSC lines share the same genetic background as their corresponding, non-edited hiPSC lines. This reduces variability associated with line-line differences in genetic background. Conditions of neurodevelopmental, neuropsychiatric and neurological disorders that have strong genetic components or are directly caused by genetic or genomic alterations can be modeled with the systems of the invention.

The methods described herein are associated with brain-region specific organoids. Brain-region specific organoids are three-dimensional (3D) aggregates of cells that resemble particular regions of the human brain and contain functional neurons that are normally associated with that region of the brain. These organoids are capable of being maintained in suspension culture for long periods of time, e.g. 2 week, 4 weeks, 1 month, 2 months, 3 months, 4 months, 5 months, 6 months or more, without adhering to a surface, e.g. a surface of a culture dish. By functional neurons, it is intended to mean that the neurons are capable of forming functional synapses with other neurons, either in the same organoid, in another organoid, or with host neurons. The formation of functional synapses can be revealed using calcium imaging, as described in more details in the Examples.

The term “neural organoid” as used herein refers to a range of brain-region specific organoids. Neural organoids encompass any organoid that is comprised of neurons from any part of the brain. Cortical organoids, midbrain organoids, striatal organoids, spinal cord/hindbrain organoids, ventral forebrain organoids, and organoids comprising any combination of the aforementioned organoids are encompassed by the term neural organoids. The terms “organoid” and “spheroid” may be used interchangeably.

The terms “anatomical integration” or “anatomically integrated” as used herein refer to neural tissue that is innervated by host neurons. The human neural tissue present within the non-human mammalian animal model comprises neurons originating from the non-human mammalian animal model nervous system and thus the human neural tissue that is anatomically integrated into the non-human mammalian animal model comprises both human neural tissue and non-human mammalian tissue.

The methods and compositions described herein are also associated with assembloids comprising more than one (e.g. two or three or more) of these brain-region specific organoids or the combination of a neural organoid and cells from another lineage (e.g., cortical organoids and microglia, pericytes, etc.). The assembloids described herein resemble multiple regions of the nervous system and contain functional neural circuits between neurons of one organoid (representing one region) and another organoid (representing another region). For example, the cortico-striatal assembloids resemble the cerebral cortex and striatum of the human brain and contain neurons (e.g. human cortical neurons) projecting from the cortical organoid into the striatal organoid, where these neurons are able functionally synapse with human striatal neurons (e.g. medium spiny neurons) of the striatal organoid. Similar to the organoids, these assembloids are also capable of being maintained for long periods of time without adhering to a surface.

Striatum. The human striatum is a region of the forebrain that is understood to act as an integrative hub for information processing in the brain and in coordinating multiple aspects of voluntary motor control. During development of the nervous system, cells of the striatum arise from the Lateral Ganglionic Eminence (LGE) of the ventral forebrain. The striatum is one of the principal components of the basal ganglia of the forebrain, a group of structures known for facilitating movement and receives inputs from the cerebral cortex, substantia nigra and thalamus. Connectivity between the cortex and striatum is unidirectional with pyramidal cortical neurons projecting into the striatum to synapse with medium spiny neurons, which are estimated to represent around 95% of neurons in the human striatum. GABAergic and cholinergic interneurons form most of the remaining population of neurons in the striatum. In addition to its widespread cortical connectivity, the striatum has extensive bidirectional connections to the midbrain.

The striatum can be divided into two main regions: the dorsal striatum and the nucleus accumbens. The dorsal striatum is associated with mediating cognition involving motor function. Dopaminergic neurons in the substantia nigra project to the dorsal striatum via the nigrostriatal pathway and regulate voluntary movement as part of the basal ganglia circuitry, where dopamine release modulates cortico-striatal transmission in medium spiny neurons expressing the dopamine receptors. The nucleus accumbens is widely associated with its role in the mesolimbic pathway associated with reward and addiction. Dopamine neurons in the ventral tegmental area of the midbrain project into the nucleus accumbens and when activated results in an increase in dopamine levels.

Medium spiny neurons are inhibitory neurons that are the principle neurons of the striatum. They are GABAergic neurons, so named due to their release of the neurotransmitter gamma-aminobutyric acid (GABA). The medium spiny neurons receive excitatory inputs from glutamatergic neurons from the cortex and is the target of dopaminergic neurons from the midbrain, where dopamine is thought to modulate the glutamatergic input.

The medium spiny neurons can be subdivided into two classes based on their projection patterns, as well as their neuropeptide and receptor expression. Medium spiny neurons that send express dopamine D1 receptors form part of the direct pathway. Medium spiny neurons that express dopamine D2 receptors form part of the indirect pathway. Classically, these two striatal medium spiny neuron populations are thought to have opposing effects on basal ganglia output. Activation of the direct medium spiny neurons has been considered to act as a ‘go’ signal to initiate behavior, whilst activation of the indirect medium spiny neurons serves as a ‘brake’ to inhibit behavior (Yager et al. 2015).

Pyramidal cortical neurons are neurons that project from the cerebral cortex to other parts of the nervous system, including the striatum. These neurons are excitatory, glutamatergic neurons.

Dopaminergic neurons are collections of neurons that produce the neurotransmitter dopamine. The neurons mainly originate in two nuclei in the human midbrain—the substantia nigra and the ventral tegmental area.

GABAergic interneurons are inhibitory neurons of the nervous system that play a vital role in neural circuitry and activity. They are so named due to their release of the neurotransmitter gamma-aminobutyric acid (GABA). An interneuron is a specialized type of neuron whose primary role is to modulate the activity of other neurons in a neural network. Cortical interneurons are so named for their localization in the cerebral cortex.

There are interneuron subtypes categorized based on the surface markers they express, including parvalbumin (PV)-expressing interneurons, somatostatin (SST)-expressing interneurons, VIP-expressing, serotonin receptor 5HT3a (5HT3aR)-expressing interneurons, etc. Although these interneurons are localized in their respective layers of the cerebral cortex, they are generated in various subpallial locations.

Morphologically speaking, cortical interneurons may be described with regard to their soma, dendrites, axons, and the synaptic connections they make. Molecular features include transcription factors, neuropeptides, calcium-binding proteins, and receptors these interneurons express, among many others. Physiological characteristics include firing pattern, action potential measurements, passive or subthreshold parameters, and postsynaptic responses, to name a few.

The PV interneuron group represents approximately 40% of the GABAergic cortical interneuron population. This population of interneurons possesses a fast-spiking pattern, and fire sustained high-frequency trains of brief action potentials. Additionally, these interneurons possess the lowest input resistance and the fastest membrane time constant of all interneurons. Two types of PV-interneurons make up the PV interneuron group: basket cells, which make synapses at the soma and proximal dendrite of target neurons, and usually have multipolar morphology and chandelier cells, which target the axon initial segment of pyramidal neurons.

The SST-expressing interneuron group is the second-largest interneuron group. SST-positive interneurons are known as Martinotti cells, and possess ascending axons that arborize layer I and establish synapses onto the dendritic tufts of pyramidal neurons. Martinotti cells are found throughout cortical layers II-VI, but are most abundant in layer V. These interneurons function by exhibiting a regular adapting firing pattern but also may initially fire bursts of two or more spikes on slow depolarizing humps when depolarized from hyperpolarized potentials. In contrast to PV-positive interneurons, excitatory inputs onto Martinotti cells are strongly facilitating.

The third group of GABAergic cortical interneurons is designated as the 5HT3aR interneuron group. VIP-expressing interneurons are localized in cortical layers II and III. VIP interneurons generally make synapses onto dendrites, and some have been observed to target other interneurons. Relative to all cortical interneurons, VIP interneurons possess a very high input resistance. In general they possess a bipolar, bitufted and multipolar morphology. Irregular spiking interneurons possess a vertically oriented, descending axon that extends to deeper cortical layers, and have an irregular firing pattern that is characterized by action potentials occurring irregularly during depolarizations near threshold, and express the calcium-binding protein calretinin (CR). Other subtypes include rapid-adapting, fast-adapting neurons IS2, as well as a minor population of VIP-positive basket cells with regular, bursting, or irregular-spiking firing patterns. Of the VIP-negative 5HT3aR group, nearly 80% express the interneuron marker Reelin. Neurogliaform cells are a type of cortical interneuron that belongs to this category: they are also known as spiderweb cells and express neuropeptide Y (NPY), with multiple dendrites radiating from a round soma.

A transcriptional network plays a role in regulating proper development and specification of GABAergic cortical interneurons, including DLX homeobox genes, LHX6, SOX6 and NKX2-1, LHX8, GSX1, GSX2. The DLX family of homeobox genes, specifically DLX1, DLX2, DLX5, and DLX6, also play a role in the specification of interneuron progenitors, and are expressed in most subpallial neural progenitor cells.

Glutamatergic neurons. The mature cerebral cortex harbors a heterogeneous population of glutamatergic neurons, organized into a highly intricate histological architecture. So-called excitatory neurons are usually classified according to the lamina where their soma is located, specific combinations of gene expression, by dendritic morphologies, electrophysiological properties, etc.

Disease relevance. Dysfunction in neural pathways from the cortex to the striatum (cortico-striatal pathway), which may also involve neural pathways to/from the midbrain, is thought to contribute to severe neuropsychiatric disorders such as schizophrenia, obsessive-compulsive disorder, Tourette syndrome, Huntington's disease, Parkinson's disease and autism spectrum disorder (ASD) (Shepherd and Gordon, 2013). As well as understanding development, the organoids and assembloids described herein are useful to model disorders of the cortico-striatal pathway as well as for testing therapeutics including gene therapy and small molecule drugs.

Schizophrenia. The systems of the present invention provide unique opportunities to study schizophrenia. Schizophrenia is a chronic and severe mental disorder that affects an individual's behavior. The underlying cause of schizophrenia is not known, but the disorder has been associated with abnormal cortical dopamine signaling (Shepherd, 2014).

Obsessive-compulsive disorder (OCD) is a mental disorder in which a person feels the need to perform certain routines repeatedly. Cortico-striatal dysfunction is considered a major factor in OCD pathogenesis and functional imaging has shown increased or otherwise abnormal functional connectivity in the cortico-striatal pathways (Shepherd, 2014). Accordingly, the systems described here provide opportunities to further study OCD and develop potential therapeutic treatments.

Tourette syndrome is a neuropsychiatric movement disorder which is clinically characterized by the presence of vocal and motor tics. Whilst the underlying cause is still unclear, various studies support a hypothesis of a dysfunction in the cortico-striatal networks as a neurobiological substrate of tics. The systems described herein therefore allow for further study of the role of the cortico-striatal networks in Tourette syndrome and support the development of treatments.

Huntington's disease is a neurodegenerative disease characterized by the progressive loss of motor and cognitive function caused by degeneration of selected neuronal populations. Huntington's disease is mainly driven by a genetic defect on chromosome 4 that results in an expanded CAG repeat at the encoding site of huntingtin protein. The neurodegenerative process in Huntington's disease mainly affects the cortex and striatum. In the striatum primarily affects the medium spiny neurons that form part of the indirect pathway. The role of these pathways in Huntington's disease and potential therapeutic treatments can be further studied using the systems described herein.

Parkinson's disease is a progressive nervous system disorder that affects movement. It develops when neurons connecting the substantia nigra in the midbrain to the striatum degenerate, resulting in a loss of dopamine signaling. There is also evidence that there is a cortico-striatal aspect to the disease (Shepherd, 2014). Accordingly, the systems described herein provide an opportunity to further study the circuits underlying Parkinson's disease and to develop new treatments.

Autism spectrum disorder (ASD) is a developmental disorder characterized by defects in social-communication and the presence of repetitive/restricted behaviors, and is associated with defects in the cortico-striatal circuits. Various studies of ASD-associated genes have demonstrated cortico-striatal involvement. Mutations in SHANK3, a postsynaptic scaffolding protein expressed in medium spiny neurons, cause the ASD-related 22q13.3 deletion syndrome, also known as Phelan-McDermid syndrome.

Timothy syndrome (TS) is characterized by multiorgan dysfunction, including severe arrhythmias, webbing of fingers and toes, congenital heart disease, immune deficiency, intermittent hypoglycemia, cognitive abnormalities, epilepsy and ASD. There are two recognized types of Timothy syndrome, classical (type-1) and atypical (type-2). They are both caused by mutations in CACNA1C, the gene encoding the calcium channel Cav1.2 a subunit. Timothy syndrome mutations in CACNA1C cause delayed channel closing, thus increased intracellular calcium. These mutations are in exon 8 (atypical form) and exon 8a (classical form), an alternatively spliced exon. Exon 8a is highly expressed in the heart, brain, gastrointestinal system, lungs, immune system, and smooth muscle. Exon 8 is also expressed in these regions and its level is roughly five-fold higher than exon 8a expression.

Tuberous sclerosis (TS) is a neurocutaneous syndrome that occurs in 1 of 6000 children; 85% of cases involve mutations in the TSC1 gene (9934), which controls the production of hamartin, or the TSC2 gene (16p13.3), which controls the production of tuberin. These proteins act as growth suppressors. If either parent has the disorder, children have a 50% risk of having it. However, new mutations account for two thirds of cases. Central nervous system (CNS) tubers interrupt neural circuits, causing developmental delay and cognitive impairment and may cause seizures, including infantile spasms. Sometimes the tubers grow and obstruct flow of cerebrospinal fluid from the lateral ventricles, causing unilateral hydrocephalus. Sometimes tubers undergo malignant degeneration into gliomas, particularly subependymal giant cell astrocytomas (SEGAs).

22q11.2 deletion syndrome (also known as DiGeorge syndrome or velocardiofacial syndrome) is a primary immunodeficiency disorder that involves T cell defects. It results from gene deletions in the DiGeorge chromosomal region at 22q11.2, which cause dysembryogenesis of structures that develop from pharyngeal pouches during the 8th week of gestation. Most cases are sporadic; boys and girls are equally affected. Inheritance is autosomal dominant. Children with DiGeorge syndrome have a specific profile in neuropsychological tests. They usually have a below-borderline normal IQ, with most individuals having higher scores in the verbal than the nonverbal domains. Some are able to attend main-stream schools, while others are home-schooled or in special classes. The severity of hypocalcemia early in childhood is associated with autism-like behavioral difficulties Adults with DiGeorge syndrome are a specifically high-risk group for developing schizophrenia. About 30% have at least one episode of psychosis and about a quarter develop schizophrenia by adulthood. Individuals with DiGeorge syndrome also have a higher risk of developing early onset Parkinson's disease (PD).

Epilepsy is a group of non-communicable neurological disorders characterized by recurrent epileptic seizures. Epileptic seizures can vary from brief and nearly undetectable periods to long periods of vigorous shaking due to abnormal electrical activity in the brain. These episodes can result in physical injuries, either directly such as broken bones or through causing accidents. In epilepsy, seizures tend to recur and may have no immediate underlying cause. Isolated seizures that are provoked by a specific cause such as poisoning are not deemed to represent epilepsy. The underlying mechanism of epileptic seizures is excessive and abnormal neuronal activity in the cortex of the brain which can be observed in the electroencephalogram (EEG) of an individual. The reason this occurs in most cases of epilepsy is unknown (idiopathic); some cases occur as the result of brain injury, stroke, brain tumors, infections of the brain, or birth defects through a process known as epileptogenesis. Known genetic mutations are directly linked to a small proportion of case.

The terms “astrocytic cell,” “astrocyte,” etc. encompass cells of the astrocyte lineage, i.e. glial progenitor cells, astrocyte precursor cells, and mature astrocytes, which for the purposes of the present invention arise from a non-astrocytic cells (i.e., glial progenitors). Astrocytes can be identified by markers specific for cells of the astrocyte lineage, e.g. GFAP, ALDH1L1, AQP4, EAAT1 and EAAT2, etc. Markers of reactive astrocytes include S100, VIM, LCN2, FGFR3 and the like. Astrocytes may have characteristics of functional astrocytes, that is, they may have the capacity of promoting synaptogenesis in primary neuronal cultures; of accumulating glycogen granules in processes; of phagocytosing synapses; and the like. A “astrocyte precursor” is defined as a cell that is capable of giving rise to progeny that include astrocytes.

Astrocytes are the most numerous and diverse neuroglial cells in the CNS. An archetypal morphological feature of astrocytes is their expression of intermediate filaments, which form the cytoskeleton. The main types of astroglial intermediate filament proteins are glial fibrillary acidic protein (GFAP) and vimentin; expression of GFAP, ALDH1L1 and/or AQP4P are commonly used as a specific marker for the identification of astrocytes.

The terms “oligodendrocyte,” “oligodendrocyte progenitor cell,” etc. can encompass cells of the oligodendrocyte lineage, i.e. neural progenitor cells that ultimately give rise to oligodendrocytes, oligodendrocyte precursor cells, and mature and myelinating oligodendrocytes, which for the purposes of the present invention arise from a non-oligodendrocyte cell by experimental manipulation. Oligodendrocytes may have functional characteristics, that is, they may have the capacity of myelinating neurons; and the like. An “oligodendrocyte precursor” or “oligodendrocyte progenitor cell” is defined as a cell that is capable of giving rise to progeny that include oligodendrocytes. Oligodendrocytes may be present in the assembloids.

Oligodendrocytes are the myelin-forming cells of the central nervous system. An oligodendrocyte extends many processes which contact and repeatedly envelope stretches of axons. Subsequent condensation of these wrapped layers of oligodendrocyte membrane form the myelin sheath. One axon may contain myelin segments from many different oligodendrocytes.

Calcium sensors. Neural activity causes rapid changes in intracellular free calcium, which can be used to track the activity of neuronal populations. Art-recognized sensors for this purpose include fluorescent proteins that fluoresce in the presence of changes in calcium concentrations. These proteins can be introduced into cells, e.g. hiPSC, by including the coding sequence on a suitable expression vector, e.g. a viral vector, to genetically modify neurons generated by the methods described herein. GCaMPs are widely used protein calcium sensors, which are comprised of a fluorescent protein, e.g. GFP, the calcium-binding protein calmodulin (CaM), and CaM-interacting M13 peptide, although a variety of other sensors are also available. Many different proteins are available, including, for example, those described in Zhao et al. (2011) Science 333:1888-1891; Mank et al. (2008) Nat. Methods 5 (9): 805-11; Akerboom et al. (2012) J. Neurosci. 32 (40): 13819-40; Chen et al. (2013) Nature 499 (7458): 295-300; etc.; and as described in U.S. Pat. Nos. 8,629,256, 9,518,980 and 9,488,642 and 9,945,844.

Optogenetics integrates optics and genetic engineering to measure and manipulate neurons. Actuators are genetically-encoded tools for light-activated control of proteins; e.g., opsins and optical switches. Opsins are light-gated ion channels or pumps that absorb light at specific wavelengths. Opsins can be targeted and expressed in specific subsets of neurons, allowing precise spatiotemporal control of these neurons by turning on and off the light source. Channel rhodopsins typically allow the fast depolarization of neurons upon exposure to light through direct stimulation of ion channels. Chlamydomonas reinhardtii Channelrhodopsin-1 (ChR1) is excited by blue light and permits nonspecific cation influx into the cell when stimulated. Examples of ChRs from other species include: CsChR (from Chloromonas subdivisa), CoChR (from Chloromonas oogama), and SdChR (from Scherffelia dubia). Synthetic variants have been created, for example ChR2 (H134R), C1V1 (t/t), ChIEF; ChETA, VChR1, Chrimson, ChrimsonR, Chronos, PsChR2, CoChR, CsChR, CheRiff, and the like. Alternatively, ChR variants that inhibit neurons have been created and identified, for example GtACR1 and GtACR2 (from the cryptophyte Guillardia theta), and variants such as iChloC, SwiChRca, Phobos, Aurora. Halorhodopsin, known as NpHR (from Natronomonas pharaoni), causes hyperpolarization of the cell when triggered with yellow light, variants include Halo, eNpHR, eNpHR2.0, eNpHR3.0, Jaws. Archaerhodopsin-3 (Arch) from Halorubrum sodomense is also used to inhibit neurons.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

### DETAILED DESCRIPTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a reprogramming factor polypeptide” includes a plurality of such polypeptides, and reference to “the induced pluripotent stem cells” includes reference to one or more induced pluripotent stem cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods for Producing Non-Human Mammalian Animal Models

As summarized above, methods are provided for producing a non-human mammalian animal model comprising human neural tissue, the method comprising introducing a first human neural organoid into a first central nervous system location of a newborn non-human mammal; and allowing the newborn non-human mammal to mature to produce the non-human mammalian animal model comprising human neural tissue.

The non-human mammalian animal may be any non-human mammalian animal. For example, the non-human mammalian animal includes, without limitation, canines; felines; equines; bovines; ovines; rodentia, such as mice or rats, etc. and primates, e.g., non-human primates, and humans. In a preferred embodiment, the non-human mammalian animal is a rat. In some embodiments, the non-human mammalian animal is newborn animal is between 1-10 days in age after birth. For instance, the newborn non-human mammalian animal may be at least 1 day old, 2 days old, 3 days old, 4 days old, 5 days old, 6 days old, 7 days, 8 days old, 9 days old, or 10 days old.

In some embodiments, the non-human mammalian animal is immunocompromised. In these embodiments, the non-human mammalian animal may be immunocompromised for any reason. For instance, the immunocompromise may be the result of including, without limitation, a genetic mutation, a chemical treatment, etc. When the immunocompromise is the result of a genetic mutation, the genetic mutation may be any mutation or set of mutations that results in immunosuppression. When the immunocompromised non-human mammalian animal is a mouse, the mouse may comprise any genetic mutation or set of genetic mutations that result in immunosuppression. For instance, the immunocompromised mouse includes, without limitation, an athymic nude mouse, a BALB/c nude mouse, CD-1 nude mouse, a Fox Chase SCID mouse, a Fox Chase SCID beige mouse, a hACE2-NCG mouse, a NCG mouse, a NOD SCID mouse, a NIH-III nude mouse, a NU/NU mouse, a SCID hairless congenic, a SCID hairless outbred mouse, a NCI SCID/NCr mouse, etc. When the immunocompromised non-human mammalian animal is a rat, the rat may comprise any genetic mutation or set of genetic mutations that result in immunosuppression. For instance, the immunocompromised rat includes, without limitation, a RNU nude rat, a SRG rat, an athymic rat, etc.

When the immunocompromise is the result of a chemical treatment, the chemical treatment may be any chemical treatment that results in immunosuppression. For instance, the chemical treatment includes, without limitation, glucocorticoids such as prednisolone, dexamethasone, etc.; cytostatic drugs such as methotrexate, cyclophosphamide, azathioprine, etc.; mycophenolate; immunophilin drugs such as rapamycin, tacrolimus, cyclosporine A, etc.; everolimus; cell therapies directed to the suppression of proliferation of specific cells such as mesenchymal stem cells, regulatory T cells, etc.; antibody treatments such as rituximab anti-thymocyte globulin, anti-lymphocyte globulin, etc.; blockage of costimulatory pathways such as CD28/B7, etc. Other known chemical treatments have been described in the art, such as in Diehl R. et al. (Cell Mol Immunol. 2017 February; 14 (2): 146-179) which has specifically been incorporated by reference.

The methods of the present disclosure comprise introducing a first human neural organoid into a non-human mammalian animal. The first human neural organoid may be any human neural organoid deemed useful. For example, the human neural organoid includes, without limitation, striatal organoids, ventral forebrain organoids, cortical organoids, midbrain organoids, spinal organoids, combinations of the aforementioned organoids, etc. In some embodiments, the human neural organoids are generated from induced human pluripotent stem cells (hiPSCs). Methods for generating hiPSCs are well known in the art and are also described below.

Human induced pluripotent stem cells. Initially, hiPSCs can be obtained from any convenient source, or can be generated from somatic cells using art-recognized methods. The hiPSCs are dissociated from feeders into single cells and grown in suspension culture, preferably when dissociated as intact colonies. In certain embodiments the culture is feeder layer free, e.g. when grown on vitronectin coated culture dishes. The culture may further be free on non-human components, i.e. xeno-free. The hiPSCs may be cultured in any medium suitable for the growth and expansion of hiPSCs. For example, the medium may be Essential 8 medium. Suspension growth optionally includes in the culture medium an effective dose of a selective Rho-associated kinase (ROCK) inhibitor for the initial period of culture, for up to about 6 hours, about 12 hours, about 18 hours, about 24 hours, about 36 hours, about 48 hours, (see, for example, Watanabe et al. (2007) Nature Biotechnology 25:681 686). Inhibitors useful for such purpose include, without limitation, Y-27632; Thiazovivin (Cell Res, 2013, 23 (10): 1187-200; Fasudil (HA-1077) HCl (J Clin Invest, 2014, 124 (9): 3757-66); GSK429286A (Proc Natl Acad Sci USA, 2014, 111 (12): E1140-8); RKI-1447; AT13148; etc. In particular embodiments the ROCK inhibitor Y-27632 is used. Optionally a WNT pathway inhibitor such as XAV-939 is added.

Human cortical spheroids. hCS may be generated by the methods previously described, for example in Pasca et al. (2015) Nat. Methods 12 (7): 671-678, entitled “Functional cortical neurons and astrocytes from human pluripotent stem cells in 3D culture” and in U.S. Pat. No. 10,494,602, each herein specifically incorporated by reference.

For example, a suspension culture of hiPS cells is cultured to provide a neural progenitor spheroid, as described above. After about 5 days, about 6 days, about 7 days, about 8 days, about 9 days, about 10 days in suspension culture, the floating neural progenitor spheroids are moved to neural media to differentiate the neural progenitors. The media is supplemented with an effective dose of FGF2 and EGF. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

To promote differentiation of neural progenitors into hCS, comprising glutamatergic neurons, after about 1 week, about 2 weeks, about 3 weeks, about 4 weeks after FGF2/EGF exposure the neural medium is changed to replace the FGF2 and EGF with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml. The cortical spheroids comprise functional glutamatergic neurons.

Human Striatal Organoids (hStrO)

Human striatal organoids may be generated by the methods previously described, for example in Miura. Y et al. (2020) Nat Biotechnol. December; 38 (12): 1421-1430, entitled “Generation of human striatal organoids and cortico-striatal assembloids from human pluripotent stem cells” and in U.S. patent application Ser. No. 17/773,429, herein specifically incorporated by reference.

To generate hStrO, after about 5 days, about 6 days, about 7 days, about 8 days, about 9 days, about 10 days in suspension culture, the floating organoids are moved to neural medium to differentiate neural progenitors. An exemplary neural medium is a medium comprising neurobasal-medium, B-27 supplement minus vitamin A and a GlutaMAX supplement. The neural medium is supplemented with an inhibitor of the Wnt pathway and a recombinant activin A. IWP-2 is an inhibitor of Wnt processing and secretion with IC50 of 27 nM in a cell-free assay, selective blockage of Porcn-mediated Wnt palmitoylation, does not affect Wnt/β-catenin in general and displays no effect against Wnt-stimulated cellular responses. The inhibitor is added, for example, at a concentration of from about 0.1 μM to about 100 μM and may be from about 1 μM to about 25 μM, depending on the activity of the inhibitor that is selected. Other inhibitors include, without limitation, XAV-939 selectively inhibits Wnt/β-catenin-mediated transcription through tankyrase1/2 inhibition with IC50 of 11 nM/4 nM in cell-free assays; ICG-001 antagonizes Wnt/β-catenin/TCF-mediated transcription and specifically binds to element-binding protein (CBP) with IC50 of 3 μM; IWR-1-endo is a Wnt pathway inhibitor with IC50 of 180 nM in L-cells expressing Wnt3A, induces Axin2 protein levels and promotes β-catenin phosphorylation by stabilizing Axin-scaffolded destruction complexes; Wnt-C59 (C59) is a PORCN inhibitor for Wnt3A-mediated activation of a multimerized TCF-binding site driving luciferase with IC50 of 74 pM in HEK293 cells; LGK-974 is a potent and specific PORCN inhibitor, and inhibits Wnt signaling with IC50 of 0.4 nM in TM3 cells; KY02111 promotes differentiation of hiPSC to cardiomyocytes by inhibiting Wnt signaling, may act downstream of APC and GSK3B; IWP-L6 is a highly potent Porcn inhibitor with EC50 of 0.5 nM; WIKI4 is a novel Tankyrase inhibitor with IC50 of 15 nM for TNKS2, and leads to inhibition of Wnt/beta-catenin signaling; FH535 is a Wnt/β-catenin signaling inhibitor and also a dual PPARγ and PPARδ antagonist.

In certain embodiments, the neural medium is supplemented with IWP-2, for example at a concentration from about 1 μM to about 10 UM, about 1 μM to about 5 μM, about 2 μM to about 3 μM, or about 2.5 μM. The recombinant activin A may be recombinant human/murine/rat activin A. In certain embodiments, the neural medium is supplemented with human/murine/rat activin A, for example at a concentration from about 10 μg/ml to about 100 μg/ml, from about 25 μg/ml to about 75 μg/ml, from about 40 μg/ml to about 60 μg/ml, or about 50 μg/ml.

To promote striatal patterning, between about 1 day and 10 days, between about 2 days and 9 days, between about 3 days and 8 days, between about 4 days and 7 days, at about 4 days, at about 5 days, at about 6 days, or at about 7 days, after exposure to the inhibitor of the Wnt pathway and the recombinant activin A, the neural medium is further supplemented with an effective dose of a retinoid X receptor (RXR) agonist. In certain embodiments, the neural medium is supplemented with an effective dose of an RXR agonist about 6 days after exposure to the inhibitor of the Wnt pathway and the recombinant activin A.

An effective dose of the RXR agonists may be included in the neural medium, for example at a concentration from about 10 μM to about 200 μM, from about 50 μM to about 150 μM, or from about 75 μM to about 125 μM, depending on the activity of the inhibitor that is selected. Exemplary agonists, without limitation SR11237, bexarotene, AGN194204, LG100268, 9-cis-retinoic acid, methoprene acid. In certain embodiments the RXR agonist is SR11237, for example added at a concentration from about 10 μM to about 200 μM, from about 50 μM to about 150 μM, from about 75 μM to about 125 μM, or about 100 μM.

As demonstrated in the examples, the combined use of an inhibitor of the Wnt pathway, a recombinant activin A and a RXR agonist results in the formation of striatal organoids with high levels of markers indicative of the human striatum, e.g. at least 3 weeks after the suspension culture of hiPSC was induced to a neural fate. For example, the striatal organoids may have high levels of forebrain markers such as FOXG1, and/or high levels of lateral ganglionic eminence (LGE) markers such as GSX2, MEIS2, CTIP2, but low levels of hypothalamus marker gene RAX and spinal cord marker gene HOXB4. Methods for determining levels of marker genes include qPCR as further described in examples. In some embodiments, the methods disclosed herein further comprise determining whether the striatal organoids express forebrain and LGE markers. A striatal organoid having high or low levels of a marker gene may have a significantly higher or lower level of marker gene expression when compared to gene expression in a non-striatal organoid, e.g. a cortical organoid (hCO), when calculated using a standard statistical test.

To promote differentiation of neural progenitors into neurons, about 1 week, about 2 weeks, about 3 weeks, about 4 weeks after exposure to the inhibitor of the Wnt pathway and the recombinant activin A the neural medium is changed to replace the inhibitor of the Wnt pathway and the recombinant activin A (and the RXR agonist, if present) with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml, or about 20 ng/ml.

The neural medium at this stage may be further supplemented with an effective dose of one or more of the following: a gamma secretase inhibitor, e.g. DAPT at a concentration of from about 1 to 25 μM, about 2 to 10 μM, and may be around about 2.5 μM; L-ascorbic acid at a concentration of from about 10 to 500 nM, from about 50 to 250 nM, and may be about 200 nM; cAMP at a concentration of from about 10 to 500 nM, from about 50 to 150 nM, and may be about 100 nM; and Docosahexaenoic acid (DHA). In some embodiments, the neural medium comprises an effective dose of BDNF, NT3, a gamma secretase inhibitor, L-ascorbic acid, CAMP and DHA.

About 4 weeks, about 5 weeks, about 6 weeks, about 7 weeks after exposure to the inhibitor of the Wnt pathway and the recombinant activin A, the organoids can be maintained for extended periods of time in neural medium, e.g., for periods of 1, 2, 3, 4, 5, 6, 7, 8, 9, 10, 11, 12 months or longer. In some embodiments, the organoids are maintained for a period of 3 months or longer. The organoids may be maintained in a neural medium in the absence of growth factors.

The human striatal organoids comprise functional GABAergic medium spiny neurons that develop dendritic spines after culture for an extended period of time in neural medium. As mentioned above, medium spiny neurons constitute the principal cell type in the human striatum and receive glutamatergic inputs from several areas including the cortex as well as dopaminergic inputs from the midbrain. The presence of dendritic spines can be determined by observation, e.g. through microscopy. The presence of GABAergic neurons can also be detected by monitoring reporter genes expressed using regulatory sequences (e.g. promoters and/or enhancers) that are specific for GABAergic neurons, such as Dlx. The functionality of the neurons can be determined by monitoring neuronal activity, e.g. by imaging Ca²⁺ activity. Midbrain Organoids.

Human midbrain organoids (hMbO) (also named midbrain organoids) may be generated by the methods previously described, for example in in U.S. patent application Ser. No. 17/773,429, herein specifically incorporated by reference.

Generation of hMbO utilizes a similar multi-step process as for hStrO generation described above, with the use of agents to promote midbrain, rather than striatal, differentiation. Early organoids induced to a neural fate by addition of effective dose of an inhibitor of BMP and of TGFβ pathways to the medium may be cultured in the presence of FGF8 and a sonic hedgehog pathway agonist, e.g. at the time of addition of the inhibitors of the BMP and TGFβ pathways, after about 12 hours, after about 24 hours, after about 1 day, after about 2 days, after about 3 days, after about 4 days of culture with the inhibitors of the BMP and TGFβ pathways. FGF8 and sonic hedgehog pathway agonist are maintained for a period of about 6 hours, about 12 hours, about 1 day, about 36 hours, about 2 days. FGF8 may be maintained for example at a concentration of from about 20 ng/ml to about 200 ng/ml, may be from about 50 ng/ml to about 150 ng/ml, may be from about 75 ng/ml to about 125 ng/ml, or may be about 100 ng/ml, depending on the activity of the inhibitor that is selected. The sonic hedgehog pathway agonist may be maintained for example at a concentration from about 0.1 μM to about 10 μM, from about 0.5 μM to about 5 μM, from about 0.5 μM to about 2 μM, or may be about 1 μM depending on the activity of the inhibitor that is selected.

Suitable sonic hedgehog pathway agonists include smoothened agonist, SAG, CAS 364590-63-6, which modulates the coupling of Smo with its downstream effector by interacting with the Smo heptahelical domain (K_{D}=59 nM). SAG may be provided in the medium at a concentration of from about 0.1 μM to about 10 μM, from about 0.5 μM to about 5 μM, from about 0.5 μM to about 2 μM, or may be about 1 μM.

The neural organoids are then moved to neural medium and cultured in the presence of FGF8, a sonic hedgehog pathway agonist, an inhibitor of BMP and an inhibitor of GSK-3. The FGF8 and sonic hedgehog pathway agonist may be as described for step (a) above. The inhibitor of BMP may be LDN-193189 (J Clin Invest, 2015, 125 (2): 796-808); Galunisertib (LY2157299) (Cancer Res, 2014, 74 (21): 5963-77); LY2109761 (Toxicology, 2014, 326C: 9-17); SB525334 (Cell Signal, 2014, 26 (12): 3027-35); SD-208; EW-7197; Kartogenin; DMH1; LDN-212854; ML347; LDN-193189 HCl (Proc Natl Acad Sci USA, 2013, 110 (52): E5039-48); SB505124; Pirfenidone (Histochem Cell Biol, 2014, 10.1007/s00418-014-1223-0); RepSox; K02288; Hesperetin; GW788388; LY364947, etc. In certain embodiments, the inhibitor of BMP is LDN-193189 provided in the medium for example at a concentration from about 10 nM to about 500 nM, from about 50 nM to about 250 nM, from about 75 nM to about 125 nM, or may be about 100 nM. The inhibitor of GSK-3 may be CHIR 99021 added for example at a concentration of from about 0.5 μM to about 50 μM, about 1 μM to about 25 μM, about 1 μM to about 10 μM, about 1 μM to about 5 μM, or may be about 3 μM.

In order to provide a midbrain organoid, the neural organoids may be cultured in the neural medium for between about 1 and 4 weeks, between about 1 and 3 weeks, between about 2 and 3 weeks, during which time the FGF8 and sonic hedgehog pathway agonist may be present in the neural medium, or may be added to the neural medium after about 1, 2, or 3 days following transfer to the neural medium. The FGF8 and sonic hedgehog pathway agonist may be maintained in the neural medium for a period of between about 7 days and about 21 days, between about 7 days and about 18 days, between about 10 days and 18 days, about 7 days, about 10 days, about 14 days, about 18 days or about 21 days in the neural medium. The inhibitor of BMP may be added to the neural medium at the same time as the FGF8 and sonic hedgehog pathway agonist and may be maintained in the neural medium for a period of between about 7 days and about 14 days, between about 7 days and about 10 days, about 7 days, about 8 days, about 9 days, or about 10 days. The inhibitor of GSK-3 may be added to the neural medium at the same time as the FGF8 and sonic hedgehog pathway agonist or may be added to the neural medium after about 1, 2, or 3 days following FGF8 and sonic hedgehog pathway agonist addition. The inhibitor of GSK-3 may be maintained in the neural medium for a period of between about 7 days and 21 days, between about 10 days and 18 days, between about 12 days and 16 days, about 7 days, about 10 days, about 15 days, or about 20 days.

In certain embodiments, the neural organoid is cultured in the neural medium for between about 2 and 3 weeks, where the inhibitor of FGF8 and sonic hedgehog pathway agonist is present in the neural medium after be present in the neural medium, or are be added to the neural medium after about 1 day, and are maintained in the neural medium for a period of between about 10 days and 18 days; the inhibitor of BMP is added to the neural medium at the same time as the FGF8 and sonic hedgehog pathway agonist, and is maintained in the neural medium for a period of between about 7 days and about 10 days; and the inhibitor of GSK-3 is added to the neural medium after about 1 day following FGF8 and sonic hedgehog pathway agonist addition, and is maintained in the neural medium for a period of between about 12 days and 16 days.

As demonstrated in the examples, the combined use of FGF8, sonic hedgehog pathway agonist, inhibitor of BMP and inhibitor of GSK-3 results in the formation of midbrain organoids with high levels of markers indicative of the human midbrain, e.g. at least 3 weeks after the suspension culture of hiPSC was induced to a neural fate. For example, the midbrain organoids may have high levels of floor plate mesencephalic dopaminergic neurons markers such as EN1 and FOXA2 and low levels of forebrain markers such as FOXG1. Methods for determining levels of marker genes include qPCR as further described in examples. In some embodiments, the methods disclosed herein further comprise determining whether the midbrain organoids express markers indicative of the human midbrain. A midbrain organoid having high or low levels of a marker gene may have a significantly higher or lower level of marker gene expression when compared to gene expression in a non-midbrain organoid, e.g. a cortical organoid, when calculated using a standard statistical test.

To promote differentiation of neural progenitors into neurons, about 1 week, about 2 weeks, about 3 weeks, about 4 weeks after exposure to FGF8, sonic hedgehog pathway agonist, inhibitor of BMP and inhibitor of GSK-3, the neural medium is changed to replace the FGF8, sonic hedgehog pathway agonist, inhibitor of BMP and inhibitor of GSK-3 with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml. The neural medium at this stage may be further supplemented with one or more of the following: a gamma secretase inhibitor, e.g. DAPT at a concentration of from about 1 to 25 μM, about 2 to 10 μM, and may be around about 2.5 μM; L-ascorbic acid at a concentration of from about 10 to 500 nM, from about 50 to 250 nM, and may be about 200 nM; CAMP at a concentration of from about 10 to 500 nM, from about 50 to 150 nM, and may be about 100 nM; and DHA.

The midbrain organoids may be maintained in the supplemented neural medium for about 1 week, about 2 weeks, about 3 weeks. After such culture, the midbrain organoids can be maintained for extended periods of time in neural medium, e.g. for periods of 1, 2, 3, 4, 5, 6, 7, 8, 9, 10, 11, 12, 24, 36 months or longer. In some embodiments, the organoids are maintained for a period of 3 months or longer. The organoids may be maintained in a neural medium in the absence of growth factors. The human midbrain organoids comprise dopaminergic neurons after culture for an extended period of time in neural medium.

Human ventral forebrain (sub-pallial) organoids (hSO or hSS). Human ventral forebrain organoids may be generated by the methods previously described, for example in Birey F. et al. (2017) Nature. May 4; 545 (7652): 54-59, entitled “Assembly of functionally integrated human forebrain organoids” and U.S. Pat. No. 10,676,715, each herein specifically incorporated by reference.

The suspension culture of hiPSC is then induced to a neural fate. This culture may be feeder-free and xeno-free. For hSO neural induction, an effective dose of an inhibitor of BMP, and of TGFβ pathways is added to the medium, for a period at least about 2 days, at least about 3 days, at least about 4 days, at least about 5 days, and up to about 10 days, up to about 9 days, up to about 8 days, up to about 7 days, up to about 6 days, up to about 5 days. For example, dorsomorphin (DM) can be added at an effective dose of at least about 0.1 μM, at least about 1 μM, at least about 5 μM, at least about 10 μM, at least about 50 μM, up to about 100 μM concentration, which inhibits bone morphogenetic protein (BMP) type I receptors (ALK2, ALK3 and ALK6). Other useful BMP inhibitors include, without limitation, A 83-01; DMH-1; K 02288; ML 347; SB 505124; etc. SB-431542 can be added at an effective dose of at least about 0.1 μM, at least about 1 μM, at least about 5 μM, at least about 10 μM, at least about 50 μM, up to about 100 μM concentration, which inhibits TGFβ signaling but has no effect on BMP signaling. Other useful inhibitors of TGFβ include, without limitation, LDN-193189 (J Clin Invest, 2015, 125 (2): 796-808); Galunisertib (LY2157299) (Cancer Res, 2014, 74 (21): 5963-77); LY2109761 (Toxicology, 2014, 326C: 9-17); SB525334 (Cell Signal, 2014, 26 (12): 3027-35); SD-208; EW-7197; Kartogenin; DMH1; LDN-212854; ML347; LDN-193189 HCl (Proc Natl Acad Sci USA, 2013, 110 (52): E5039-48); SB505124; Pirfenidone (Histochem Cell Biol, 2014, 10.1007/s00418-014-1223-0); RepSox; K02288; Hesperetin; GW788388; LY364947, A 83-01, etc.

Early organoids patterned by SMAD inhibition, e.g. at the time of transfer to the SMAD inhibitory medium, after about 12 hours, after about 24 hours, after about 1 day, after about 2 days, after about 3 days, after about 4 days, are cultured in the presence of an effective dose of a Wnt inhibitor and an SHH inhibitor in the culture medium. The Wnt and SHH inhibitors are maintained for a period of about 7 days, about 10 days, about 14 days, about 18 days, about 21 days, about 24 days, for example at a concentration of from about 0.1 μM to about 100 μM, and may be from about 1 μM to about 50 μM, from about 5 μM to about 25 μM, etc. depending on the activity of the inhibitor that is selected.

Exemplary WNT inhibitors include, without limitation, XAV-939 selectively inhibits Wnt/β-catenin-mediated transcription through tankyrase ½ inhibition with IC50 of 11 nM/4 nM in cell-free assays; ICG-001 antagonizes Wnt/β-catenin/TCF-mediated transcription and specifically binds to element-binding protein (CBP) with IC50 of 3 μM; IWR-1-endo is a Wnt pathway inhibitor with IC50 of 180 nM in L-cells expressing Wnt3A, induces Axin2 protein levels and promotes β-catenin phosphorylation by stabilizing Axin-scaffolded destruction complexes; Wnt-C59 (C59) is a PORCN inhibitor for Wnt3A-mediated activation of a multimerized TCF-binding site driving luciferase with IC50 of 74 pM in HEK293 cells; LGK-974 is a potent and specific PORCN inhibitor, and inhibits Wnt signaling with IC50 of 0.4 nM in TM3 cells; KY02111 promotes differentiation of hPSCs to cardiomyocytes by inhibiting Wnt signaling, may act downstream of APC and GSK3B; IWP-2 is an inhibitor of Wnt processing and secretion with IC50 of 27 nM in a cell-free assay, selective blockage of Porcn-mediated Wnt palmitoylation, does not affect Wnt/β-catenin in general and displays no effect against Wnt-stimulated cellular responses; IWP-L6 is a highly potent Porcn inhibitor with EC50 of 0.5 nM; WIKI4 is a novel Tankyrase inhibitor with IC50 of 15 nM for TNKS2, and leads to inhibition of Wnt/beta-catenin signaling; FH535 is a Wnt/β-catenin signaling inhibitor and also a dual PPARγ and PPARδ antagonist.

SHH agonists include smoothened agonist, SAG, CAS 364590-63-6, which modulates the coupling of Smo with its downstream effector by interacting with the Smo heptahelical domain (K_{D}=59 nM). SAG may be provided in the medium at a concentration of from about 10 nM to about 10 μM, from about 50 nM to about 1 μM, from about 75 nM to about 500 nM, and may be around about 100 nM.

Optionally the medium in this stage of the hSO culture process further comprises allopregnanolone from about day 10 to about day 23, e.g. from day 11, 12, 13, 14, 15, 16, 17, 18, 19, 20, 21, 22 until the conclusion of the stage; at a concentration of from about 10 nM to about 10 μM, from about 50 nM to about 1 μM, from about 75 nM to about 500 nM, and may be around about 100 nM.

Optionally the hSO cultures are transiently exposed to retinoic acid, e.g. for about 1 to about 4 days, which may be from about day 10 to about day 20, from about day 12 to about day 15, etc., at a concentration of from about 10 nM to about 10 μM, from about 50 nM to about 1 μM, from about 75 nM to about 500 nM, and may be around about 100 nM.

For hSO conditions, after about 5 days, about 6 days, about 7 days, about 8 days, about 9 days, about 10, after about 15 days, after about 20 days, after about 25 days, e.g., around about 23 days, in suspension culture, the floating organoids are moved to neural media to differentiate neural progenitors. The media is supplemented with an effective dose of FGF2 and EGF. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

To promote differentiation of neural progenitors into neurons, after about 1 week, about 2 weeks, about 3 weeks, about 4 weeks after FGF2/EGF exposure the neural medium is changed to replace the FGF2 and EGF with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

After about 4 weeks, about 5 weeks, about 6 weeks, about 7 weeks after FGF2/EGF exposure, the spheroids can be maintained for extended periods of time in neural medium in the absence of growth factors, e.g. for periods of 1, 2, 3, 4, 5, 6, 7, 8, 9, 10, 11, 12 months or longer. The number of astrocytes in the cultures are initially low for the first month, and increase in number after that, up to from about 5%, about 10%, about 15%, about 20%, about 25%, to about 30% or more of the cells in the organoids.

Human spinal organoids (hSpO or hSpS). Human spinal organoids may be generated by the methods previously described, for example in Anderson, J. et al. (2020) Nature. December 23; 183 (7): 1913-1929.e26, entitled “Generation of Functional Human 3D Cortico-Motor Assembloids” and U.S. patent application Ser. No. 17/253,038, each herein specifically incorporated by reference.

To generate the SpOs, hiPSCs are dissociated and grown in suspension; then induced to a neural fate by SMAD inhibitors, e.g. dorsomorphin at a concentration of from about 1 to 50 mM, about 2 to 25 mM, and may be around about 5 mM; and SB-431542 at a concentration of from about 2 to 100 mM, about 5 to 50 mM, and may be around about 10 mM. The cells are cultured in this medium for periods of from about 2 to about 5 days, and may be about 4 days; after which time the medium is supplemented with a GSK-3 inhibitor, e.g. CHIR 99021 at a concentration of from about 1 to 50 mM, about 2 to 25 mM, and may be around about 3 mM. The cells are maintained in the medium for an addition 1 to 3 days, and may be maintained for 2 days. CHIR may be maintained until day 18, or may be removed after day 6.

The cells are then moved to neural medium in the presence of retinoic acid at a concentration of from about 10 to 1 mM, from about 50 to 150 nM, and may be about 100 nM, FGF2 at a concentration of from about 0 to 50 ng/ml, from about 2.5 to 25 ng/ml and may be about 10 ng/ml; and EGF at a concentration of from about 1 to 50 ng/ml, from about 2.5 to 25 ng/ml and may be about 20 ng/ml, for a period of from about 3 to 7 days, and may be around about 5 days. The medium is then supplemented with an SHH pathway agonist, e.g. smoothened agonist (SAG) at a concentration of from about 0 to 1 mM, from about 50 to 150 nM, and may be about 100 nM. After about 5 to 9 days, e.g. after about 7 days, the medium is optionally supplemented with gamma secretase inhibitor, e.g. DAPT at a concentration of from about 1 to 25 mM, about 2 to 10 mM, and may be around about 2.5 mM, which supplement may be provided one, two, three or more times at intervals of from about 1 to 3 days. This completes the fate specification stage. Concentrations of RA and FGF2 may be titrated to achieve different rostro-caudal positions within the spinal cord (which may be determined by expression of HOX genes, with HOX4-HOX8 being cervical/brachial and HOX9-HOX11 being thoracic/lumbar). Concentrations of SAG may be titrated to achieve different dorso-ventral positions within the spinal cord (which may be determined by expression of PAX3 and OLIG2, among others). The spheroids may then be maintained in culture in neural medium supplemented with BDNF at a concentration of from about 1 to 50 ng/ml, from about 2.5 to 25 ng/ml and may be about 20 ng/ml; IGF at a concentration of from about 1 to 50 ng/ml, from about 2.5 to 25 ng/ml and may be about 10 ng/ml, L-ascorbic acid at a concentration of from about 10 to 500 nM, from about 50 to 250 nM, and may be about 200 nM; and cAMP at a concentration of from about 10 to 500 nM, from about 50 to 150 nM, and may be about 62.5 nM. After such culture, the spheroids can be maintained for extended periods of time in neural medium in the absence of growth factors, e.g. for periods of 1, 2, 3, 4, 5, 6, 7, 8, 9, 10, 11, 12, 24, 36 months or longer.

Spinal cord organoids (hSpO) comprise functional human cholinergic motor neurons; and excitatory and inhibitory interneurons. The relative proportion of these types of neurons can be shifted by modulating the Notch pathway through the optional inclusion of DAPT in the medium as described above. These functional motor neurons have the ability to promote muscle contractions in skeletal muscle cells.

Assembloids. The hStrO can be functionally integrated with separately cultured human cortical organoids (hCS), to form cortico-striatal assembloids (hCO-hStrO) which include glutamatergic neurons. The resulting hCO-hStrO contains cortico-striatal circuits and provides for functional integration of these circuits. Functionally integrated cells interact in a physiologically relevant manner, e.g. forming synapses or neuromuscular junctions, transmitting signals, forming multicellular structures, and the like.

The cortical organoids are co-cultured with the human striatal organoids in neural medium under conditions permissive for cell fusion. Condition permissive for cell fusion may include culturing the hStrO and hCO in close proximity, e.g. in direct contact with one another.

Assembly may be performed with organoids after around about 30 days, about 60 days, about 90 days of culture for hStrO; and after around about 30 days, about 60 days, about 90 days of culture for hCO. The hStrO and hCO organoids may be co-cultured for a period of 3 days, 5 days, 8 days, 10 days, 14 days, 18 days, 21 days or more. The resulting cortico-striatal assembloids are demonstrated to contain functional neural circuits, where the assembloids comprise glutamatergic neurons projecting from the hCO to the hStrO. The glutamatergic neurons may be unidirectional neurons, for example unidirectional CTIP2 and/or SATB2 expressing neurons. Methods for confirming the functionality of the neurons are known in the art and include optogenetic methods and imaging of calcium activity in neurons, such as those methods described in the examples. In some embodiments, the methods may comprise confirming the functionality of the neurons in the cortico-striatal assembloid.

Human Midbrain Organoids (hMbO) and Midbrain-Striatal Assembloids (hMbO-hStrO)

As described above, the human striatum receives dopaminergic input from the midbrain, which plays an important role in the development and maturation of the striatum. Additionally, GABAergic modulation of the midbrain from the striatum is an essential component of the basal ganglia direct pathway. In order to study these circuits, the hMbO can be functionally integrated with separately cultured hStrO to form midbrain-striatal assembloids (hMbO-hStrO), which include dopaminergic neurons and GABAergic medium spiny neurons. The resulting hMbO-hStrO assembloids provide for functional integration of the midbrain-striatal circuits.

The hMbO and hStrO are generated separately as described herein and then co-cultured in neural medium under conditions permissive for cell fusion. Condition permissive for cell fusion may include culturing the hMbO and hStrO in close proximity, e.g. in contact with one another.

Assembly may be performed with organoids after around about 30 days, about 60 days, about 90 days of culture for hStrO; and after around about 30 days, about 60 days, about 90 days of culture for hMbO. The hStrO and hMbO organoids may be co-cultured for a period of 1 day, 2 days, 3 days or more. The resulting hMbO-hStrO contain functional neural circuits, where the hMbO-hStrO assembloids comprise midbrain neurons (e.g. dopaminergic neurons) projecting from hMbO to hStrO.

The organoids described above can be further assembled into three-part cortico-striatal-midbrain assembloids (hCO-hStrO-hMbO) to study neural circuits involving neurons of the striatum, cortex and midbrain. For example, the hCO-hStrO assembloids described above can be co-cultured with hMbO to provide these three-part assembloids. Alternatively, the hMbO-hStrO described above can be co-cultured with hCO to provide the three-part assembloids. Further alternatively, the hMbO, hStrO and hCO organoids can be separately generated and all three organoids co-cultured to provide the three-part assembloids. The resulting three-part assembloids comprise dopaminergic neurons projecting from hMbO to hStrO and glutamatergic neurons projecting from the hCO to hStrO and can be used to study glutamatergic and dopaminergic modulation in this system.

In some embodiments, the neural organoids of the present disclosure are isolated from an individual who is predicted to have or has been diagnosed with a neuropsychiatric disorder. The neuropsychiatric disorder may be any neuropsychiatric disorder that is deemed suitable for organoid culture. Neuropsychiatric disorders that find use in the present disclosure include, with limitation, Timothy syndrome, tuberous sclerosis, 22q11.2 deletion syndrome (also known as DiGeorge syndrome), Autism spectrum disorder, Epilepsy, Schizophrenia, Huntington's disease, Parkinson's disease, Tourette's syndrome, etc.

The methods of embodiments of the present disclosure comprise introducing a first human neural organoid into a first central nervous system location. The first human neural organoid may be introduced in a variety of ways. In some embodiments, introducing comprises employing an introducer loaded with the neural organoid. The introducer may be any introducer that can be loaded with neural organoid. Introducers that find use in the present disclosure include, without limitation, a syringe, an auto-injector, a tube, a pipette, a pipette tip, a needle, etc.

In some embodiments, the introducing comprises making access to a central nervous system location. In some embodiments, making access to a first central nervous system location comprises performing a craniotomy. In some embodiments, performing a craniotomy further comprises perforating the dura of the brain. In some embodiments, when the dura is perforated, a first central nervous system location, e.g. a specific brain region, is contacted with an introducer wherein the introducer is retracted upon contact and a neural organoid is deposited at the site of contact. The first human neural organoid may be introduced into any location within the central nervous system deemed useful including, without limitation, a brain, a spinal cord, etc. When a human neural organoid is introduced into a brain, it may be in any suitable location within the brain. For instance, the human neural organoid may introduced to a specific region of the brain including, without limitation, the frontal cortex, the motor cortex, somatosensory cortex, parietal cortex, occipital cortex, temporal cortex, the cerebellum, spinal cord, etc. In some embodiments, the location within the brain is specific for the type of organoid used. For instance, cortical organoids may be introduced to the somatosensory cortex whereas ventral forebrain organoids may be introduced to the frontal cortex. In some embodiments, the location with the brain is not specific for the type of organoid used.

In some embodiments, the methods further comprise introducing a second human neural organoid into a second central nervous system location. In some embodiments, the second human neural organoid is the same as the first human neural organoid. For example, if the first human neural organoid is a cortical organoid then the second human neural organoid is a cortical organoid. In some embodiments, the second human neural organoid is different from the first human neural organoid. In this instance, the first human neural organoid may be a cortical organoid and the second human neural organoid may be a midbrain organoid, a striatal organoid, a ventral forebrain organoid or any other neural organoid that is not a cortical organoid. In some embodiments, the second central nervous system location is the same as the first central nervous system location. For example, when the first human neural organoid is introduced into the frontal cortex then the second human neural organoid is also introduced into the frontal cortex. In some embodiments, the second central nervous system location is the same as the first central nervous system location but is in the opposite brain hemisphere. In some embodiments, the second central nervous system location is different from the first central nervous system location. In these embodiments, if the first central nervous system location is the frontal cortex then the second central nervous system location is any other central nervous system location that is not the frontal cortex such as the motor cortex, somatosensory cortex, parietal cortex, occipital cortex, temporal cortex, spinal cord or the cerebellum.

Following the introduction of the first human neural organoid into the first central nervous system location of the newborn non-human mammalian animal, the newborn non-human mammal is allowed to mature thereby producing a non-human mammalian animal model comprising human neural tissue. The newborn non-human mammalian animal may be allowed to mature for any amount of time deemed suitable. Suitable amounts of time to allow for the newborn non-human mammalian animal to mature includes, without limitation, 5-10 days, 10-15 days, 15-20 days, 20-25 days, 25-30 days, 30-35 days, 35-40 days, 40-45 days, 45-50 days, 50-60 days, 60-70 days, 70-80 days, 80-90 days, 90-100 days, 100-110 days, 110-120 days, 120-130 days, 130-140 days, 140-150 days, 150-160 days, 160-170 days, 170-180 days, 180-190 days, 190-200 days, 200-210 days, 210-220 days, 220-230 days, 230-240 days, 240-250 days, or greater than 250 days. The amount of time to allow for the newborn non-human mammalian animal to mature may be at any intervening amount of time within a specific range. For instance, when the amount of time to allow for the newborn non-human mammalian animal to mature is from 80-90 days, the amount of time may be 90 days, 91 days, 92 days, 93 days, 94 days, 95 days, 96 days, 97 days, 98 days, 99 days, or 100 days.

In some embodiments, the non-human mammalian animal model comprises anatomically integrated human neural tissue. In some embodiments, the anatomical integration of the human neural tissue does not result in discernable locomotor or memory deficits in the non-human mammalian animal model. In some embodiments, the anatomically integrated human neural tissue is vascularized. In some embodiments, the anatomically integrated human neural tissue comprises deep and superficial layer glutamatergic neurons, cycling progenitors, oligodendrocytes and astrocytes. In some embodiments, the non-human mammalian animal model comprising anatomically integrated human neural tissue receives physiological sensory input from said human neural tissue. The physiological sensory input can be in the form of any sensory input including, without limitation, touch, sight, hearing, taste, smell, etc.

### Methods for Modeling Neuropsychiatric Disorders

As summarized above, methods are provided for modeling a neuropsychiatric disorder, the method comprising introducing a first human neural organoid produced from a cellular biological sample from an individual living with a neuropsychiatric disorder into a central nervous system location of a newborn non-human mammal; allowing the newborn non-human mammal to mature to produce the non-human mammalian animal model comprising a first human neural tissue; and characterizing the first human neural tissue to model the neuropsychiatric disorder.

The first human neural organoid produced from the cellular biological sample from an individual living with a neuropsychiatric disorder may be produced using any method deemed useful. In some embodiments, the first human neural organoid is produced by converting cells of the cellular biological sample into induced pluripotent stem cells; and differentiating the induced pluripotent stem cells into the first human neural organoid.

The neuropsychiatric disorder may be any neuropsychiatric disorder that is deemed suitable for organoid culture. Neuropsychiatric disorders that find use in the present disclosure include, with limitation, Timothy syndrome, tuberous sclerosis, 22q11.2 deletion syndrome (also known as DiGeorge syndrome), Autism spectrum disorder, Epilepsy, Schizophrenia, Huntington's disease, Parkinson's disease, Tourette's syndrome, etc.

The neuropsychiatric disorders of the present disclosure may be characterized in a variety of ways. In some embodiments, the characterizing comprises measuring the neuronal morphology of the first human neural tissue. In these embodiments, a number of different neuronal morphological features can be measured. For instance, the morphological features that can be measured include, without limitation, soma diameter, dendrite number, dendrite length, dendrite density, dendritic spine number, dendritic spine length, dendritic spine density, axon length, etc. The neuronal morphology may be measured in a variety of ways. For example, the measuring includes, without limitation, histologically staining the first human neural tissue, antibody staining the first human neural tissue, expressing a detectably labeled protein in the first human neural tissue, etc.

Histological stains that find use in the present disclosure include, without limitation, H&E staining, Nissl staining, Luxol-fast blue staining, Kluver-Barrera staining, Bodian silver staining, Holzer staining, Gallyas-Braak staining, thionine staining, Weil-Myelin staining, Solochrome staining, Perls staining, Fluoro-Jade staining, Congo Red staining, thioflavine S staining, amino cupric silver staining, Neutral Red Counter staining, cupric silver staining, Campbell-Switzer Alzheimer staining, autometallography staining, etc. Antibody stains that find use in the present disclosure include, without limitation, 4G8, 6E10, AB1-40, AB1-42, alpha synuclein, Asyn-pSer129, AT8, BrdU+hematoxylin, calbindin, caspase-3, caspase-9, cathepsin-D, CD68, c-fos, ChAT+Nissl, doublecortin, endoglin, ferritin, GAD-67, GFAP, GFP, HulgG, Iba1, Ki-67, LAMP1, luciferase, MAP-2, MBP, mDectin, NeuN, Nestin, Oligo2, Orexin A, parvalbumin, p-c-jun, P.U.1, RGMa, S830, SMI-71, SMI-99, somatostatin, STEM-101, TDP-43, TH, TMEM119, TPH, etc. The first human neural tissue of the present disclosure may also be antibody stained or immunostained for reasons other than histology. For instance, the first human neural tissue may be antibody stained or immunostained for a number of reasons including, without limitation, to determine the presence or absence of a protein, to determine the differential expression of a protein, to determine the localization of a protein, etc.

A detectably labeled protein of the present disclosure may be any protein labeled with a detectable moiety. Detectable moieties may include, without limitation, a fluorescent protein, a luminescent protein, etc. For instance, the marker protein may be a fluorescent protein or a luminescent protein. Non-limiting examples of useful fluorescent proteins include but are not limited to GFP, EBFP, Azurite, Cerulean, mCFP, Turquoise, ECFP, mKeima-Red, TagCFP, AmCyan, mTFP, TurboGFP, TagGFP, EGFP, TagYFP, EYFP, Topaz, Venus, mCitrine, TurboYFP, mOrange, TurboRFP, tdTomato, TagRFP, dsRed2, mRFP, mCherry, mPlum mRaspberry, mScarlet, etc. Examples of luminescent proteins, include without limitation, Cypridinia luciferase, Gaussia luciferase, Renilla luciferase, Phontinus luciferase, Luciola luciferase, Pyrophorus luciferase, Phrixothrix luciferase, etc.

In some embodiments, the characterizing comprises measuring intrinsic electrophysical properties of the first human neural tissue. Any intrinsic electrophysical properties of the first human neural tissue may be measured. Non-limiting examples of electrophysical properties that may be measured include, without limitation, resting membrane potential, depolarization threshold, membrane capacitance, maximal firing rates, minimum firing rates, etc. Intrinsic electrophysical properties may be measured using a multitude of techniques including, without limitation, sharp electrodes, patch-clamp, fluorescent bioelectricity reporters, etc.

In some embodiments, the characterizing comprises measuring gene expression in the first human neural tissue. The expression of any gene may be measured, particularly genes related to neural development or neural function. The expression of a single gene, sets of genes, the transcriptome, or the proteome may be measured. The expression of genes may be measured in the form or mRNA or protein.

In some embodiments, the characterizing comprises axon tracing of the first human neural tissue. The axons may be traced in any way deemed useful. The axon tracing may be retrograde tracing or anterograde tracing. The axon tracing may be performed with the use of a viruses, protein or small molecule. Non-limiting examples of viruses that facilitate anterograde axon tracing include, without limitation, herpes simplex virus 1 (HSV-1), HSV-1 strain H129, rhabdoviruses, etc. Non-limiting examples of viruses that facilitate retrograde axon tracing include, without limitation, rabies, pseudorabies, glycoprotein, deleted rabies, etc. Non-limiting examples of proteins and small molecules that facilitate anterograde axon tracing include, without limitation, Phaseolus vulgaris-leucoagglutinin, wheat germ agglutin, dextran amines, etc. Non-limiting examples of proteins and small molecules that facilitate retrograde axon tracing include, without limitation, horse radish peroxidase (HRP), wheat germ agglutin, cholera toxin subunit B, hydroxystilbamidine, Fast Blue, Diamidino Yellow, True Blue, the, carbocyanines DiI and DiO, fluorescent lax microspheres, etc. Non-limiting examples of proteins and small molecules that facilitate retrograde axon tracing include, without limitation, horse radish peroxidase (HRP), wheat germ agglutin, cholera toxin subunit B, hydroxystilbamidine, Fast Blue, Diamidino Yellow, True Blue, the, carbocyanines DiI and DiO, fluorescent lax microspheres, etc. Other viruses, proteins and small molecules that facilitate axon tracing have been described in the art, for example, in Xu, X. et al. (Neuron. 2020 Sep. 23; 107 (6): 1029-1047) and in Saleeba, C. et al. (Front Neurosci. 2019 Aug. 27; 13:897), each of which herein specifically incorporated by reference.

In some embodiments, the method of modeling a neuropsychiatric disorder further comprises characterizing the non-human mammalian animal model. In these embodiments, characterizing the non-human mammalian animal model may include, without limitation, assaying behavioral responses to tasks, memory responses to tasks, motor responses to tasks, sensory responses to tasks, etc. Nonlimiting examples of behavioral tests that find use in the present disclosure include, without limitation, Acoustic Startle Response, Elevated Plus Maze, Elevated Zero Maze, Foot Placement Analysis, Forced Exercise/Walking, Forced Swim Test in Mice, Hot Plate Test, Hargreaves Test, Grid Walking Test, Water Maze, Vertical Screen Test, Tail Suspension Test, Rota Rod Test, Resident Intruder, Passive Avoidance Test, Oxymax System, etc. Memory tests that find use in the present disclosure include, without limitation, Water Maze, Passive Avoidance Test, Novel Object Recognition, etc. Nonlimiting examples of motor tests that find use in the present disclosure include, without limitation, Acoustic Startle Response, Balance Beam, Bar Holding Test, Analysis of Locomotion Using CatWalk Test, Foot Placement Analysis, Forced Exercise/Walking, Forced Swim Test in Mice, Grid Walking Test, Vertical Screen Test, Rota Rod Test, Neurological Exam, Locomotor Activity Test, etc. Sensory tests that find use in the present disclosure include, without limitation, Acoustic Startle Response, Cold Plate Test, Hot Plate Test, Hargreaves Test, Neurological Exam, Mechanical Sensitivity (Von Frey), etc. The above disclosed tests are well known in the art and have been previously described in, for example, Buccafusco, J. et al. (Methods of Behavior Analysis in Neuroscience. 2nd edition. Boca Raton (FL): CRC Press/Taylor & Francis; 2009.) in addition to others, herein specifically incorporated by reference.

In some embodiments, the method further comprises introducing a second human neural organoid into a second central nervous system location wherein the second human neural organoid is derived from an individual that does not have a neuropsychiatric disorder. In some embodiments, the second human neural organoid is the same as the first human neural organoid except that it is derived from an individual that does not have a neuropsychiatric disorder. For example, if the first human neural organoid is a cortical organoid then the second human neural organoid is a cortical organoid. In some embodiments, the second human neural organoid is different from the first human neural organoid. In this instance, the first human neural organoid may be a cortical organoid and the second human neural organoid may be a midbrain organoid, a striatal organoid, a ventral forebrain organoid or any other neural organoid that is not a cortical organoid. In some embodiments, the second central nervous system location is the same as the first central nervous system location. For example, when the first human neural organoid is introduced into the frontal cortex then the second human neural organoid is also introduced into the frontal cortex. In some embodiments, the second central nervous system location is the same as the first central nervous system location but is in the opposite brain hemisphere. In some embodiments, the second central nervous system location is different from the first central nervous system location. In these embodiments, if the first central nervous system location is the frontal cortex then the second central nervous system location is any other central nervous system location that is not the frontal cortex such as the motor cortex, somatosensory cortex, parietal cortex, occipital cortex, temporal cortex, spinal cord or the cerebellum.

In embodiments where the second human neural organoid is introduced into a second central nervous system location, the second human neural organoid produces a second human neural tissue. In these embodiments, the method further comprises characterizing the second human neural tissue. The second human neural tissue is used as a control or reference to compare to the first human neural tissue. The results of the characterizing of the first human neural tissue may then be compared to the results of characterizing the second human neural tissue such that the differences between the first and second human neural tissue may be determined.

### Methods for Determining the Effectiveness of a Drug on a Neuropsychiatric Disorder

Also disclosed herein are methods for determining the effectiveness of a candidate agent on a neuropsychiatric disorder, the method comprising administering the candidate agent to the non-human mammalian animal model comprising human neural tissue produced using the methods for modeling a neuropsychiatric disorder; assaying the human neural tissue; and comparing the results of the assaying with mammals administered a control agent that is not the candidate agent. A candidate agent may be a chemical, small molecule, a protein, a genetic agent or an antibody. In some embodiments, the candidate agent is administered systemically in the non-human mammalian animal model. In some embodiments, the candidate agent is administered locally at the site of the human neural tissue.

The assaying of the human neural tissue may involve determining the effect a candidate agent has on the neuronal morphology, intrinsic electrophysical properties, gene expression, immunostaining, axon tracing, and/or intracellular calcium levels of the neurons within the human neural tissue. As described herein, the human neural tissue is anatomically integrated into the non-human mammalian animal model and this human neural tissue and the neurons contained therein are functional. The assaying may therefore involve determining whether a candidate agent is able to alter the neuronal morphology, intrinsic electrophysical properties, gene expression, immunostaining and/or axon tracing of the human neural tissue derived from an individual having a neuropsychiatric disorder such that these features more closely resemble that of human neural tissue that is derived from an individual that does not have a neuropsychiatric disorder.

As also described herein, various diseases and disorders are associated with neural dysfunction. Accordingly, the assays described herein may find particular utility where the human neural tissue is derived from an individual having a neuropsychiatric disorder, such as Timothy syndrome, tuberous sclerosis, 22q11.2 deletion syndrome (also known as DiGeorge syndrome), Autism spectrum disorder, Epilepsy, Schizophrenia, Huntington's disease, Parkinson's disease, Tourette's syndrome, etc. Candidate agents that are able to restore the functionality of defects or abnormalities identified in the human neural tissue comprising the neuropsychiatric disorder may have therapeutic utility in the treatment of said disorder.

Neural activity causes rapid changes in intracellular free calcium. Calcium imaging assays that exploit this can therefore be used to determine the functionality of the anatomically integrated neuronal circuits of the human neural tissue. For example, detected changes in calcium levels with a cell or cluster of cells would indicate a change in activity in said cells. This may involve modifying the human neural organoids that the human neural tissue was derived from to contain genetically-encoded calcium indicator proteins, such those proteins that include the fluorophore sensor GCaMP and imaging those cells. GCaMP comprises a circularly permuted green fluorescent protein, a calcium-binding protein calmodulin (CaM) and CaM-interacting M13 peptide, where brightness of the GFP increases upon calcium binding. Further details about calcium imaging assays are described in Chen et al. (2013) Nature 499 (7458): 295-300. Other calcium imaging assays include Fura-2 calcium imaging; Fluo-4 calcium imaging, and Cal-590 calcium imaging.

In some embodiments, the methods further comprise assaying the non-human mammalian animal model comprising human neural tissue to determine the effect the candidate agent has on the behavioral responses to tasks, memory responses to tasks, motor responses to tasks, sensory responses to tasks. The assaying may also therefore involve determining whether a candidate agent is able to alter the behavioral responses to tasks, memory responses to tasks, motor responses to tasks, sensory responses to tasks such that these responses more closely resemble that of a non-human mammalian animal model comprising human neural tissue that is derived from an individual that does not have a neuropsychiatric disorder.

In some embodiments, the non-human mammalian animal model comprises human neural tissue derived from a second human neural organoid introduced into the second central nervous system location wherein the second human neural organoid derived from an individual that does not have a neuropsychiatric disorder. In these embodiments, the human neural tissue that is produced from the first human neural organoid that is derived from an individual having a neuropsychiatric disorder is referred to as the first human neural. In these embodiments, the results of the assaying of the human neural tissue derived from an individual having a neuropsychiatric disorder may be compared to results of assaying the human neural tissue, e.g. the second human neural tissue, that is derived from an individual that does not have a neuropsychiatric disorder. The results of the assaying of the first human neural tissue may then be compared to the results of assaying the second human neural tissue such that the differences between the first and second human neural tissue may be determined both in response to the candidate agent and to a control agent.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, New York, (1996), Ninth edition, under the sections: Drugs Acting at Synaptic and Neuroeffector Junctional Sites; Cardiovascular Drugs; Vitamins, Dermatology; and Toxicology, all incorporated herein by reference.

Test compounds include all of the classes of molecules described above and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also include the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome, for example using CRISPR mediated genomic engineering (see for example Shmakov et al. (2017) Nature Reviews Microbiology 15:169). Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in human neural tissue lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc. The expression vector may be a viral vector, e.g. adeno-associated virus, adenovirus, herpes simplex virus, retrovirus, lentivirus, alphavirus, flavivirus, rhabdovirus, measles virus, Newcastle disease virus, poxvirus and picornavirus vectors.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′—NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs.

The results of an assay can be entered into a data processor to provide a dataset. Algorithms are used for the comparison and analysis of data obtained under different conditions. The effect of factors and agents is read out by determining changes in multiple parameters e.g. the neuronal morphology, intrinsic electrophysical properties, gene expression, immunostaining and/or axon tracing of the human neural tissue. The data will include the results from assay combinations with the agent(s), and may also include one or more of a control human neural tissue (i.e. human neural tissue produced from a human neural organoid derived from an individual that does not have a neuropsychiatric disorder, the human neural tissue derived from an individual having a neuropsychiatric disorder), and the results from other assay combinations using other agents or performed under other conditions. For rapid and easy comparisons, the results may be presented visually in a graph, and can include numbers, graphs, color representations, etc.

The dataset may be prepared from values obtained by measuring parameters in the presence and absence of different stimuli, e.g. a visual stimuli, a touch stimuli, a taste stimuli, a smell stimuli, a auditory stimuli, as well as comparing the presence of the agent of interest and at least one other state, usually the control state, which may include the state without agent or with a different agent. The parameters include functional states such as synapse formation and calcium ions in response to stimulation, whose levels vary in the presence of the factors, neuronal morphology, intrinsic electrophysical properties, gene expression, immunostaining and/or axon tracing of the human neural tissue. Desirably, the results are normalized against a standard, usually a “control value or state,” to provide a normalized data set such as results obtained from human neural tissue derived from an individual that does not have a neuropsychiatric disorder or unstimulated human neural tissue derived from an individual that has a neuropsychiatric disorder. Values obtained from test conditions can be normalized by subtracting the unstimulated control values from the test values, and dividing the corrected test value by the corrected stimulated control value. Other methods of normalization can also be used; and the logarithm or other derivative of measured values or ratio of test to stimulated or other control values may be used. Data is normalized to control data on the same cell type under control conditions, but a dataset may comprise normalized data from one, two or multiple human neural tissues and assay conditions.

The dataset can comprise values of the levels of sets of parameters obtained under different assay combinations. Compilations are developed that provide the values for a sufficient number of alternative assay combinations to allow comparison of values.

A database can be compiled from sets of experiments, for example, a database can contain data obtained from a panel of assay combinations, with multiple different environmental changes, where each change can be a series of related compounds, or compounds representing different classes of molecules.

Mathematical systems can be used to compare datasets, and to provide quantitative measures of similarities and differences between them. For example, the datasets can be analyzed by pattern recognition algorithms or clustering methods (e.g. hierarchical or k-means clustering, etc.) that use statistical analysis (correlation coefficients, etc.) to quantify relatedness. These methods can be modified (by weighting, employing classification strategies, etc.) to optimize the ability of a dataset to discriminate different functional effects. For example, individual parameters can be given more or less weight when analyzing the dataset, in order to enhance the discriminatory ability of the analysis. The effect of altering the weights assigned each parameter is assessed, and an iterative process is used to optimize pathway or cellular function discrimination.

The comparison of a dataset obtained from a test compound, and a reference dataset(s) is accomplished by the use of suitable deduction protocols, AI systems, statistical comparisons, etc. Preferably, the dataset is compared with a database of reference data. Similarity to reference data involving known pathway stimuli or inhibitors can provide an initial indication of the cellular pathways targeted or altered by the test stimulus or agent.

A reference database can be compiled. These databases may include reference data from panels that include known agents or combinations of agents that target specific pathways, as well as references from the analysis of human neural tissue treated under environmental conditions in which single or multiple environmental conditions or parameters are removed or specifically altered. Reference data may also be generated from panels containing human neural tissue with genetic constructs that selectively target or modulate specific cellular pathways. In this way, a database is developed that can reveal the contributions of individual pathways to a complex response.

The effectiveness of pattern search algorithms in classification can involve the optimization of the number of parameters and assay combinations. The disclosed techniques for selection of parameters provide for computational requirements resulting in physiologically relevant outputs. Moreover, these techniques for pre-filtering data sets (or potential data sets) using cell activity and disease-relevant biological information improve the likelihood that the outputs returned from database searches will be relevant to predicting agent mechanisms and in vivo agent effects.

For the development of an expert system for selection and classification of biologically active drug compounds or other interventions, the following procedures are employed. For every reference and test pattern, typically a data matrix is generated, where each point of the data matrix corresponds to a readout from a parameter, where data for each parameter may come from replicate determinations, e.g. multiple individual human neural tissues of the same type. A data point may be quantitative, semi-quantitative, or qualitative, depending on the nature of the parameter.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Classification rules are constructed from sets of training data (i.e. data matrices) obtained from multiple repeated experiments. Classification rules are selected as correctly identifying repeated reference patterns and successfully distinguishing distinct reference patterns. Classification rule-learning algorithms may include decision tree methods, statistical methods, naive Bayesian algorithms, and the like.

A knowledge database will be of sufficient complexity to permit novel test data to be effectively identified and classified. Several approaches for generating a sufficiently encompassing set of classification patterns, and sufficiently powerful mathematical/statistical methods for discriminating between them can accomplish this.

The data from human neural tissue treated with specific drugs known to interact with particular targets or pathways provide a more detailed set of classification readouts. Data generated from human neural tissues that are genetically modified using over-expression techniques and anti-sense techniques, permit testing the influence of individual genes on the phenotype.

A preferred knowledge database contains reference data from optimized panels of human neural tissues, environments and parameters. For complex environments, data reflecting small variations in the environment may also be included in the knowledge database, e.g. environments where one or more factors or human neural tissue types of interest are excluded or included or quantitatively altered in, for example, concentration or time of exposure, etc.

### Methods for Altering the Behavior of a Mammal

Methods are provided for altering the behavior of a mammal, the method comprising introducing a first human neural organoid into a first central nervous system location of a newborn non-human mammal; allowing the newborn non-human mammal to mature to produce a non-human mammal comprising a first human neural tissue; implanting an optical fiber into the first human neural tissue; training the mammal on an operant conditioning paradigm using the optical fiber; and stimulating the first human neural tissue using the optical fiber, wherein the first human neural tissue comprises a first light activatable polypeptide, wherein stimulating the first human neural tissue results in a change in the activity of the neurons in said first human neural tissue; to alter the behavior of the mammal.

The non-human mammal may be any non-human mammal discussed above. The first human neural organoid and the first central nervous system location may be any of the human neural organoids and any central nervous system locations discussed above.

The optical fibers of the present disclosure may be any optical fiber that is capable of transmitting multiple light wavelengths. In some embodiments, the light wavelength is red light from about 625 nm to about 740 nm. In some embodiments, the light wavelength is orange light from about 590 nm to about 625 nm. In some embodiments, the light wavelength is yellow light from about 565 nm to about 590 nm. In some embodiments, the light wavelength is green light from about 520 nm to about 565 nm. In some embodiments, the light wavelength is blue light from about 445 nm to about 520 nm. In some embodiments, the light wavelength is indigo light from about 425 nm to about 445 nm. In some embodiments, the light wavelength is violet light from about 380 nm to about 425 nm.

The operant conditioning may be any conditioning in which a specific behavior is either positively or negatively reinforced when said behavior is performed following stimulation using the optical fiber. In this paradigm, the non-human mammal is placed in a behavioral testing chamber and the first human neural tissue is stimulated using the optical fiber with interleaved first and second light wavelengths for set intervals of time. The non-human mammal is presented with a reward, punishment, or a combination thereof when performing the behavior when the first human neural tissue is stimulated with a first or second light wavelength. Stimulation with the first or the second light wavelength results in a change in the activity of the first human neural tissue. Following a period of time in which the operant conditioning is performed, the non-human mammal behavior is altered in response to stimulation with the first or second light wavelength.

In some embodiments, the non-human mammal is then presented with a reward when the behavior is performed when the first human neural tissue is stimulated with the first light wavelength and is not presented with a reward when the first human neural tissue is stimulated with the second light wavelength. In some embodiments, the non-human mammal is presented with a punishment when the behavior is performed when the first human neural tissue is stimulated with the second light wavelength and is not presented with a punishment when the first human neural tissue is stimulated with the first light wavelength. In some embodiments, the non-human mammal is presented with a reward when performing the behavior when the first human neural tissue is stimulated with the first light wavelength and is punished when performing the behavior when the first human neural tissue is stimulated with the second wavelength of light.

The first and second light wavelength may be any of the light wavelengths discussed above. In some embodiments, the first light wavelength is blue light and the second wavelength of light is red light. The set interval of light stimulation between interleaved first and send light wavelengths can be an interval deemed used useful. For instance, the interval may be 1 second(s), 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s or greater than 30 s.

The operant conditioning may be performed for any length of time deemed necessary. In general, the time deemed necessary would be a period of time in which the non-human mammal behavior is either increased or decreased when the first human neural tissue is stimulated with the first or the second wavelength of light. For instance, the operant conditioning may be performed for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks or greater than 10 weeks.

The activity of the first human neural tissue is altered as a result of the activation of the first light activatable polypeptide present in the first human neural tissue. The first light activable polypeptide may either depolarize or hyperpolarize the neurons in the first human neural tissue. Non-limiting examples of light activatable polypeptides capable of mediating a hyperpolarizing current can be found, e.g., in International Patent Application No. PCT/US2011/028893; U.S. Pat. No. 9,175,095. Non-limiting examples of hyperpolarizing light-activatable polypeptides include NpHr, eNpHr2.0, eNpHr3.0, eNpHr3.I or GtR3. Non-limiting examples of depolarizing light activatable polypeptides include “C1V1”, channel rhodopsin 1 (ChR1), VChR1, channel rhodopsin 2 (ChR2). Additional information regarding other light-activated cation channels, anion pumps, and proton pumps can be found in U.S. Patent Application Publication Nos: 2009/0093403; and International Patent Application No: PCT/US2011/028893.

In some embodiments, the non-human mammal further comprises introducing a second human neural organoid into a second central nervous system location of a newborn non-human mammal wherein when the newborn non-human animal matures the second human neural organoid produces a second human neural tissue. In some embodiments, the second human neural organoid is the same as the first human neural organoid. For example, if the first human neural organoid is a cortical organoid then the second human neural organoid is a cortical organoid. In some embodiments, the second human neural organoid is different from the first human neural organoid. In this instance, the first human neural organoid may be a cortical organoid and the second human neural organoid may be a midbrain organoid, a striatal organoid, a ventral forebrain organoid or any other neural organoid that is not a cortical organoid. In some embodiments, the second central nervous system location is the same as the first central nervous system location. For example, when the first human neural organoid is introduced into the frontal cortex then the second human neural organoid is also introduced into the frontal cortex. In some embodiments, the second central nervous system location is the same as the first central nervous system location but is in the opposite brain hemisphere. In some embodiments, the second central nervous system location is different from the first central nervous system location. In these embodiments, if the first central nervous system location is the frontal cortex then the second central nervous system location is any other central nervous system location that is not the frontal cortex such as the motor cortex, somatosensory cortex, parietal cortex, occipital cortex, temporal cortex, spinal cord or the cerebellum.

In these embodiments, the second human neural tissue comprises a second light activatable polypeptide. In some embodiments, the second light activatable polypeptide is the same as the first light activatable polypeptide. In some embodiments, the second light activatable polypeptide is different from the first light activatable polypeptide. In some embodiments, the second light activatable polypeptide hyperpolarizes the second human neural tissue and the first light activatable polypeptide depolarizes the first human neural tissue. In some embodiments, the second light activatable polypeptide depolarizes the second human neural tissue and the first light activatable polypeptide hyperpolarizes the first human neural tissue. In some embodiments, the second light activatable polypeptide hyperpolarizes the second human neural tissue and the first light activatable polypeptide hyperpolarizes the first human neural tissue. In some embodiments, the second light activatable polypeptide depolarizes the second human neural tissue and the first light activatable polypeptide depolarizes the first human neural tissue.

### Methods for Treating a Neuropsychiatric Disorder

Methods are provided for treating a neuropsychiatric disorder, the method comprising introducing a human neural organoid into a central nervous system location of a newborn mammal; allowing the newborn mammal to mature to produce the mammal comprising human neural tissue wherein the human neural tissue corrects or prevents the development of the neuropsychiatric disorder.

The methods disclosed herein involve the treatment of a neuropsychiatric disorder. Neuropsychiatric disorders that may be treated by the methods comprise a central nervous system abnormality as the result of a genetic mutation or developmental defect. In these embodiments, the human neural organoid is derived from an individual that does not have the genetic mutation associated with the neuropsychiatric disorder. The human neural organoid is then transplanted into a central nervous system location in a newborn mammal. The transplanted human neural organoid then develops into human neural tissue thereby correcting or preventing the central nervous system abnormality from occurring.

In some embodiments, the human neural organoid is transplanted to a central nervous system location that is specific to the neuropsychiatric disorder. In these embodiments, the neuropsychiatric disorder leads to a structural or functional abnormality in a specific region of the central nervous system. In some embodiments, the human neural organoid is transplanted to a central nervous system location that is not specific to the neuropsychiatric disorder. In these embodiments, the neuropsychiatric disorder does not cause a structural or functional abnormalities that is restricted to a specific region within the central nervous system.

In some embodiments, the mammal is a rodent. In some embodiments, the rodent is a rat. In some embodiments, the rodent is a mouse. In some embodiments, the mammal is a non-human primate. In some embodiments, the mammal is a human.

In some embodiments, the method further comprises screening the newborn mammal prior to treatment. In some embodiments, the newborn mammal is screened in utero. In some embodiments, the newborn mammal is screened after birth. The screening may be any screening that is able to identify the neuropsychiatric disorder or a propensity to develop said neuropsychiatric disorder. In some embodiments, the screening involves genetic testing. In these embodiments, the individual is genetically tested for a specific genetic sequence related to or associated with a neuropsychiatric disorder. In some embodiments, the screening involves visualizing the central nervous system. The visualizing may be any visualizing technique that allows the imaging of central nervous system including, without limitation, ultrasound, magnetic resonance imaging, computed tomography scan, etc. The visualization of the central nervous system then allows identification of structural abnormalities within the central nervous system.

For further elaboration of general techniques useful in the practice of this invention, the practitioner can refer to standard textbooks and reviews in cell biology, tissue culture, embryology, and neurobiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

Each publication cited in this specification is hereby incorporated by reference in its entirety for all purposes.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Experimental

### Introduction

Human brain development is a remarkable self-organizing process in which cells proliferate, differentiate, migrate, and wire to form functioning neural circuits that are subsequently refined by sensory experience (Kelley, K. W. et al. Cell. 2022 Jan. 6; 185 (1): 42-61). A critical challenge to understanding brain development, particularly in the context of disease, is a lack of access to human brain tissue. By applying instructive signals to human induced pluripotent stem cells (hiPSC) grown in tridimensional (3D) cultures, the generation of self-organizing organoids resembling specific brain regions was previously shown, including human cortical spheroids (hCS) (Pasca, A. M. et al. Nat Methods. 2015 July; 12 (7): 671-8). hCS recapitulate certain features of the cerebral cortex (Pasca, A. M. et al. Nat Methods. 2015 July; 12 (7): 671-8; Yoon, S. J. et al. Nat Methods. 2019 January; 16 (1): 75-78), including specification of cortical progenitors, neurons and astrocytes, and they can be assembled with other organoids to study cell migration (Birey, F. et al. Nature. 2017 May 4; 545 (7652): 54-59); however, there are several limitations that restrict their broader applications in understanding neural circuit development and function. Specifically, it is currently unclear to what extent hCS neuronal maturation in vitro is constrained by the lack of the specific microenvironments and sensory input that exist in vivo. Moreover, since hCS are not integrated into circuits that can generate behavioral outputs, their utility in modeling genetically complex and behaviorally-defined neuropsychiatric diseases is currently limited.

Transplantation of hCS into intact living brains has the potential to overcome these limitations. Indeed, prior studies have demonstrated that human neurons transplanted into the rodent cortex survive, project and make connections with rodent cells (Espuny-Camacho, I. et al. Neuron. 2013 Feb. 6; 77 (3): 440-56; Linaro, D. et al. Neuron. 2019 Dec. 4; 104 (5): 972-986.e6; Mansour, A. A. et al. Nat Biotechnol. 2018 June; 36 (5): 432-441; Real, R. et al. Science. 2018 Nov. 16; 362 (6416): eaau1810; Kitahara, T. et al. Stem Cell Reports. 2020 Aug. 11; 15 (2): 467-481; Xiong, M. et al. Cell Stem Cell. 2021 Jan. 7; 28 (1): 112-126.e6). However, these experiments have typically been performed in adult animals which likely limits synaptic and axonal integration. Here, a novel transplantation paradigm is introduced in which 3D hCS derived from hiPSC are transplanted into the somatosensory cortex of immunodeficient rats at an early, plastic stage of development. Neurons from transplanted hCS (t-hCS) undergo significant maturation. Importantly, advanced maturation in t-hCS reveals defects in neurons derived from patients with a severe, genetic disease caused by a mutation in the L-type voltage-sensitive calcium channel Cav1.2 (CACNA1C) that mediates calcium influx in neurons to initiate activity-dependent gene transcription (Ebert, D. H. et al. Nature. 2013 Jan. 17; 493 (7432): 327-37). Transplanted hCS (t-hCS) receive thalamocortical and cortico-cortical inputs that are capable of evoking sensory responses and extend axonal projections into the rat brain that can drive reward-seeking behaviors. Importantly, advanced maturation in t-hCS reveals defects in neurons derived from patients with a severe, genetic disease caused by a mutation in the L-type voltage-sensitive calcium channel Cav1.2 (CACNA1C) that mediates calcium influx in neurons to initiate activity-dependent gene transcription (Ebert, D. H. et al. Nature. 2013 Jan. 17; 493 (7432): 327-37).

### Results

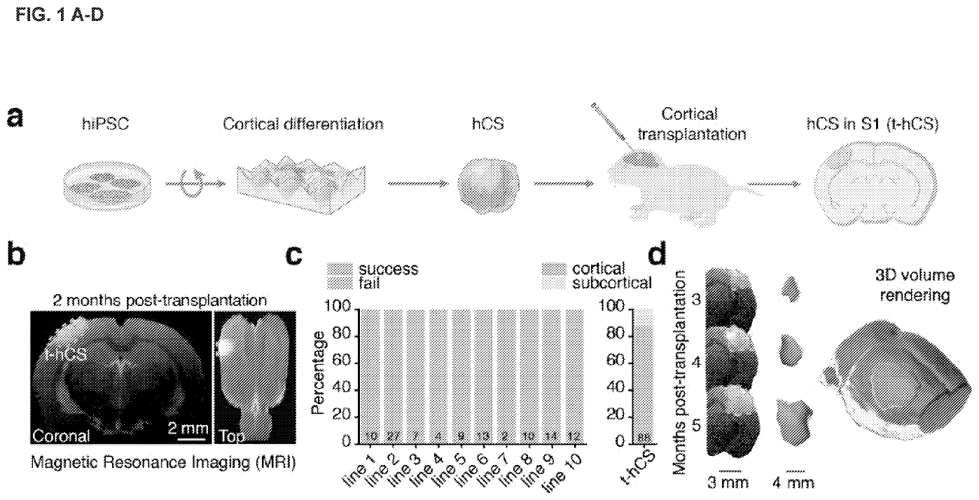

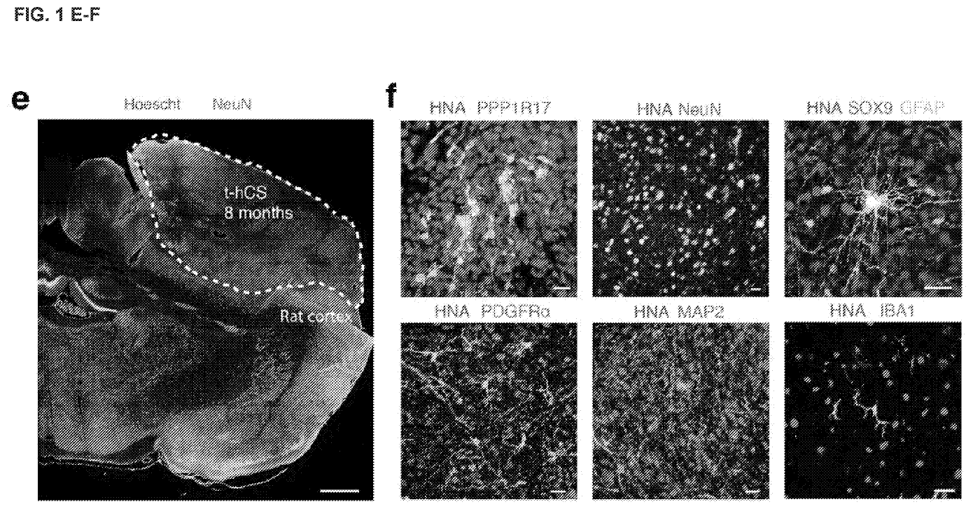

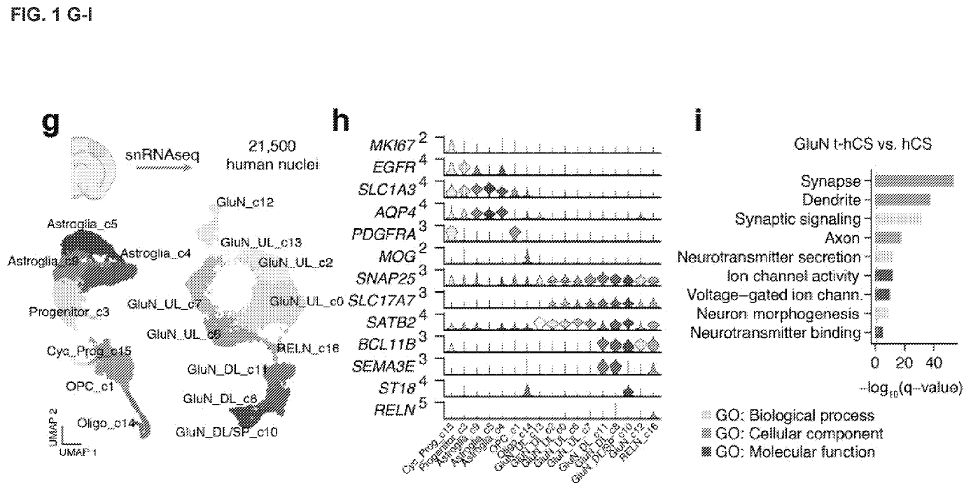

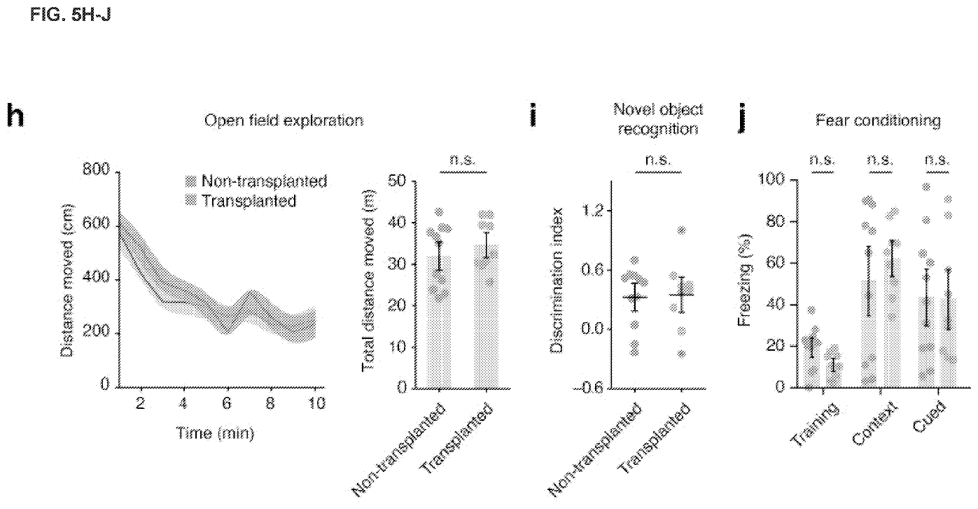

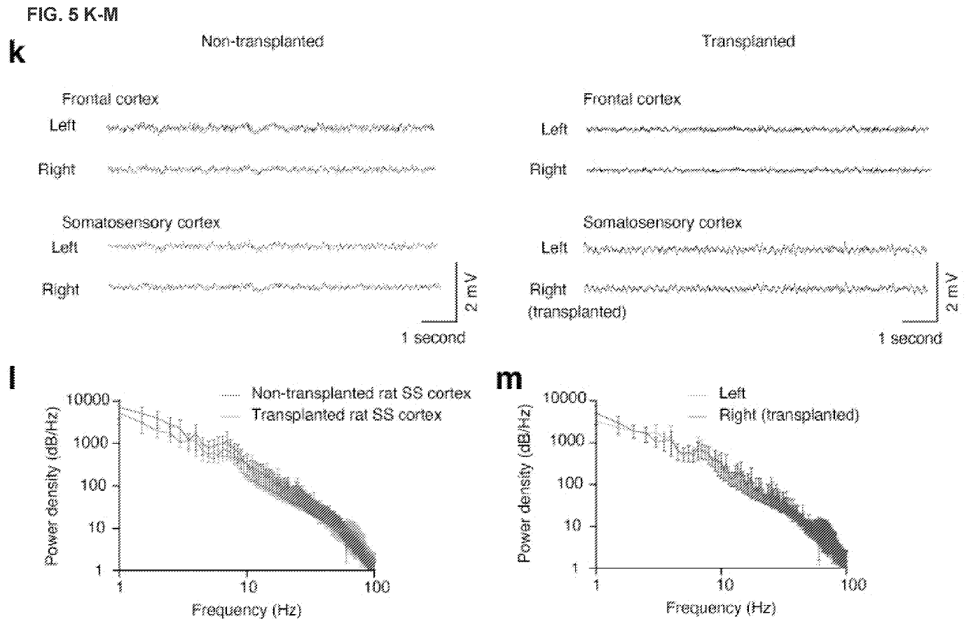

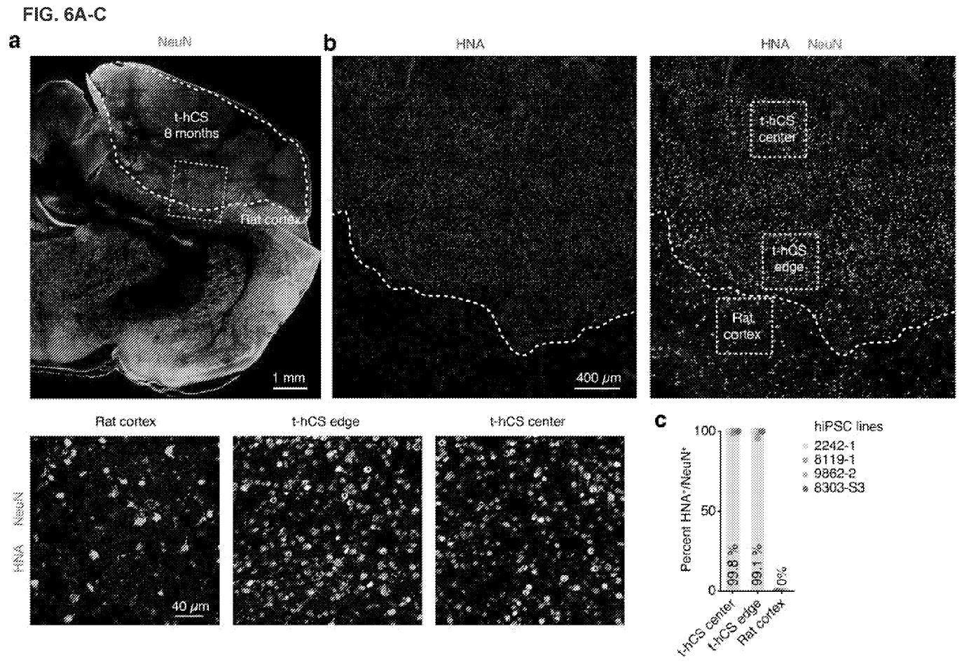

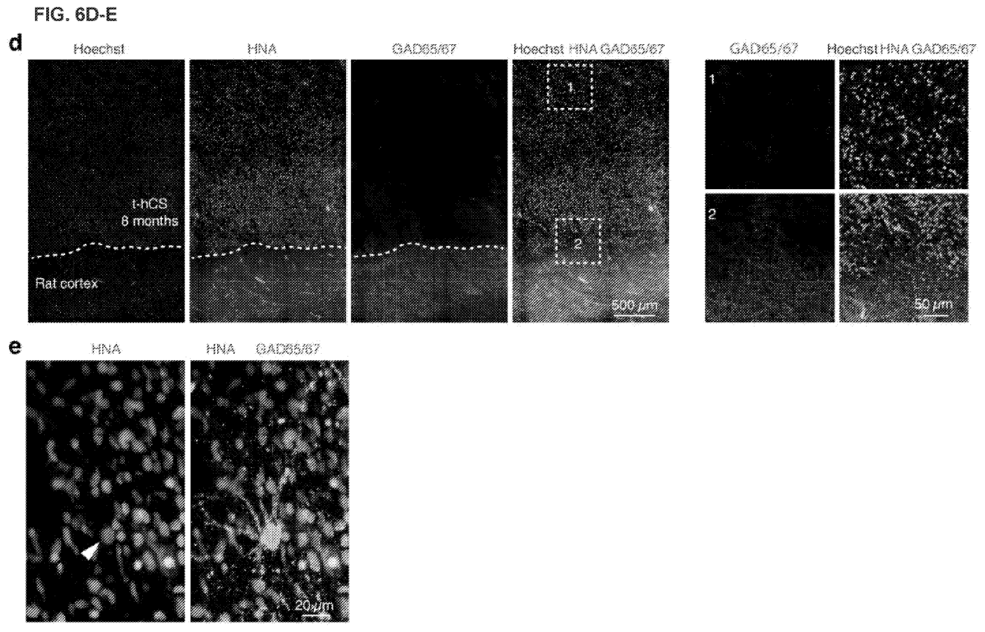

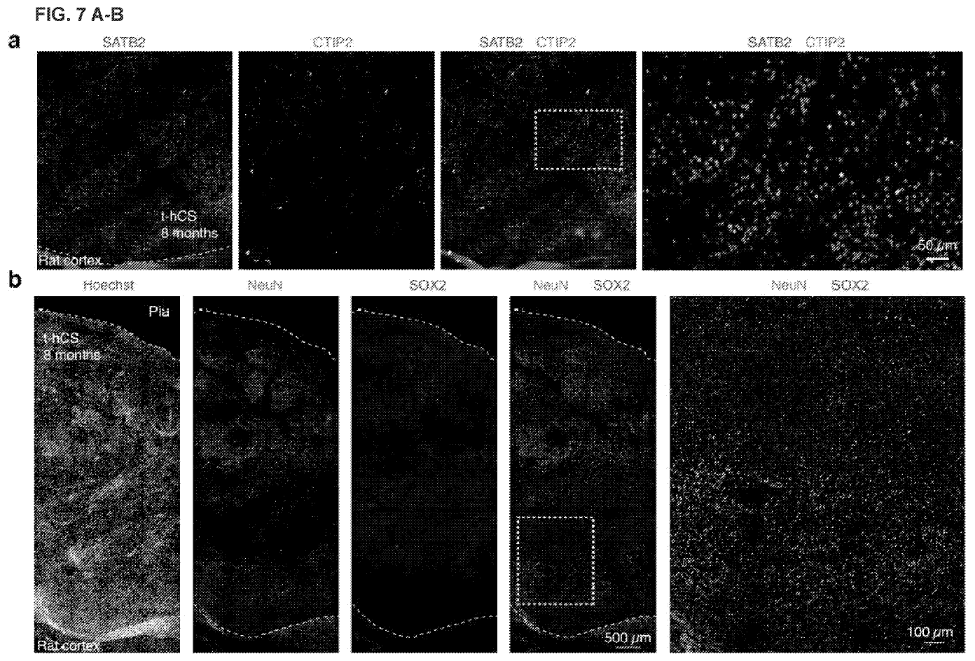

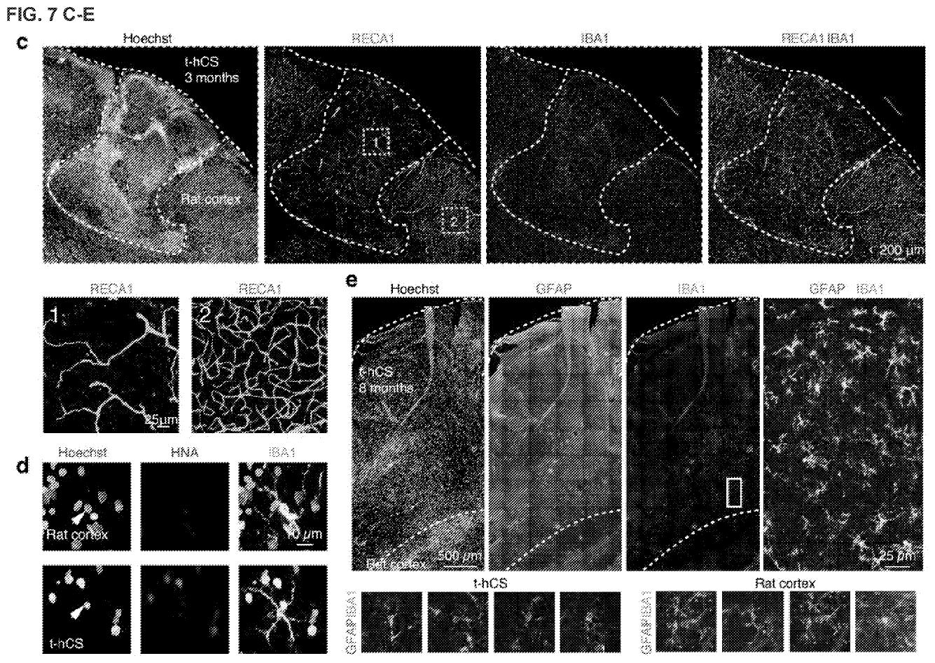

A protocol for generating hCS that recapitulate several features of the cerebral cortex in vitro was previously described. To advance this platform to allow the study of human-derived neurons within in vivo circuits that possess both sensory inputs and behavioral outputs intact 3D hCS were stereotactically transplanted into the primary somatosensory cortex (S1) of early postnatal athymic rats (P3-P7) (FIG. 1a, FIG. 5a-c) rats rather than mice were used which are smaller and would make transplantation challenging, and the engraftment was performed at a developmental stage at which thalamocortical and corticocortical axonal projections have not yet completed their innervation of S1 (Kichula, E. A. et al. J Comp Neurol. 2008 Jul. 20; 509 (3): 239-58). This approach therefore aims to maximize t-hCS localization and integration, while minimally compromising endogenous circuitry. To visualize t-hCS location in living animals, performed T2-weighted MRI reconstructions of the rat brain was performed at 2-3 months post-transplantation (FIG. 1b, FIG. 5d). t-hCS were readily observed and t-hCS volume measurements were highly correlated with those calculated in 3D reconstructed grafts from fixed slices (FIG. 1d & e, P>0.05). Using this approach, identified t-hCS were identified in 81% of engrafted animals at ˜2 months post-transplantation (n=72 animals; hCS derived from n=10 hiPSC lines). Of these, the great majority (87%) were located in the cerebral cortex (FIG. 1c). Finally, by performing consecutive MRI scans and 3D volume reconstructions of the engrafted rat brain at multiple timepoints in the same rats, it was found that t-hCS increased 9-fold in volume over the span of 3 months (FIG. 1d, FIG. 5f, ** P<0.01). Importantly, the survival rate of transplanted animals was high even after 12 months from transplantation (74%) (FIG. 5g), and no discernable locomotor or memory deficits, gliosis, or EEG abnormalities were detected in these animals (FIG. 5h-m, FIG. 7b).

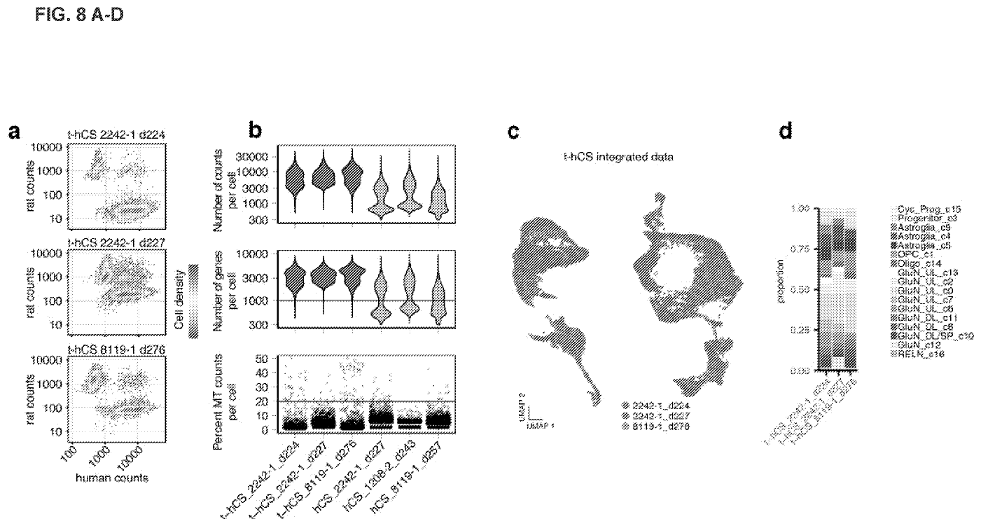

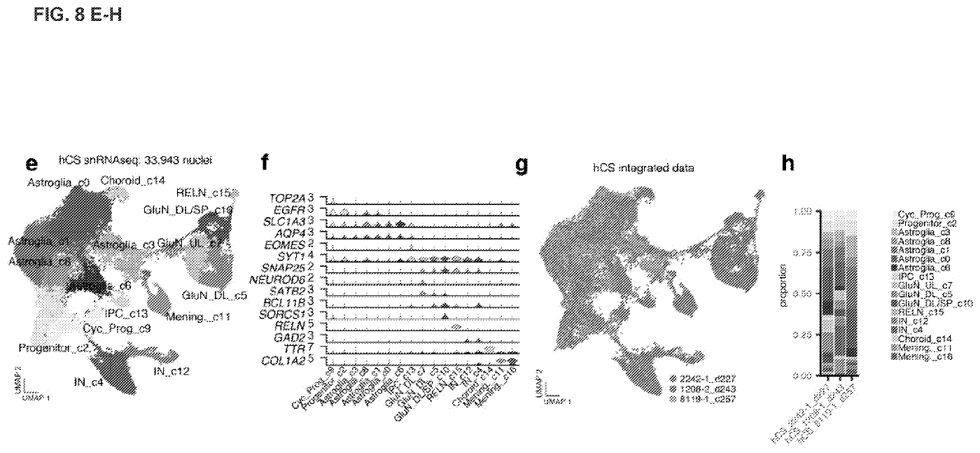

The cytoarchitecture and gross cellular composition of t-hCS was next assessed. Antibody staining for the rat endothelial marker Reca-1 revealed vascularization of t-hCS, while staining for Iba1 revealed the presence of rat microglia throughout the graft (FIG. 7). Immunostainings further identified human nuclear antigen (HNA) positive cells that co-expressed PPP1R17 (cortical progenitors), NeuN (neurons), SOX9 and GFAP (astrocytes), or PDGFRα (oligodendrocytes) (FIG. 1f). To explore the cellular composition of t-hCS with single cell resolution, single-nucleus RNA sequencing (snRNA-seq) was performed from two samples at ˜8 months of differentiation. Computational removal of rat nuclei in addition to quality filtering yielded 15,023 high-quality human single-nucleus profiles (FIG. 1g, FIG. 8a, b). Expression patterns of canonical cell type markers identified clusters of major cortical cell classes, including both deep and superficial layer glutamatergic neurons, cycling progenitors, oligodendrocytes, and astrocytes (FIG. 1g, h). Cell type proportions were concordant across t-hCS replicates (FIG. 8c, d). Single-nucleus RNA-seq of comparably aged hCS yielded broadly similar cell classes with the exception of the presence of GABAergic neurons in vitro and myelinating oligodendrocyte transcripts in vivo (FIG. 8e, f). Differential gene expression analysis highlighted substantial differences in glutamatergic neurons between t-hCS and hCS. Gene sets associated with neuronal maturation, including synaptic signaling, dendrite localization, and voltage-gated channel activity were highly enriched in genes with increased expression in t-hCS glutamatergic neurons (FIG. 1i). Thus, t-hCS maintains high cellular diversity and leads to increased transcriptional maturation of cortical glutamatergic neurons.

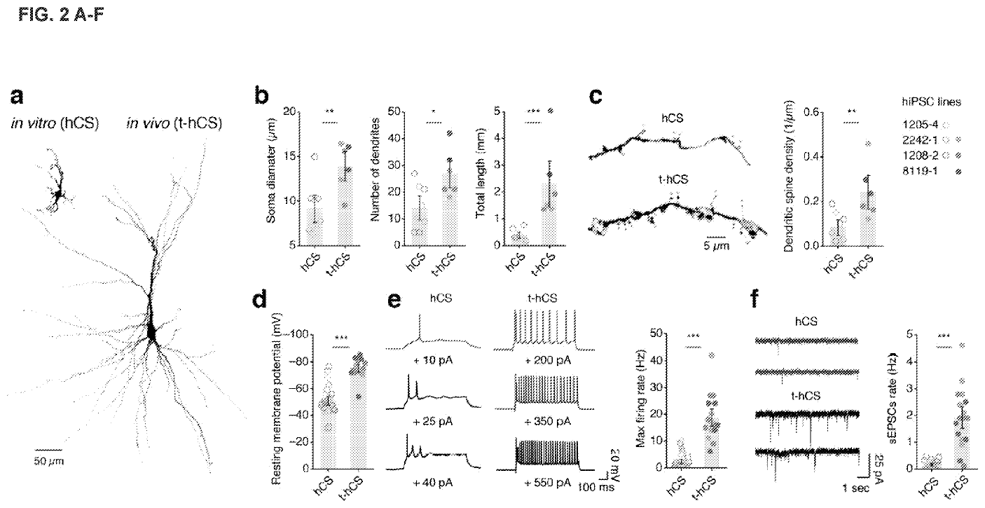

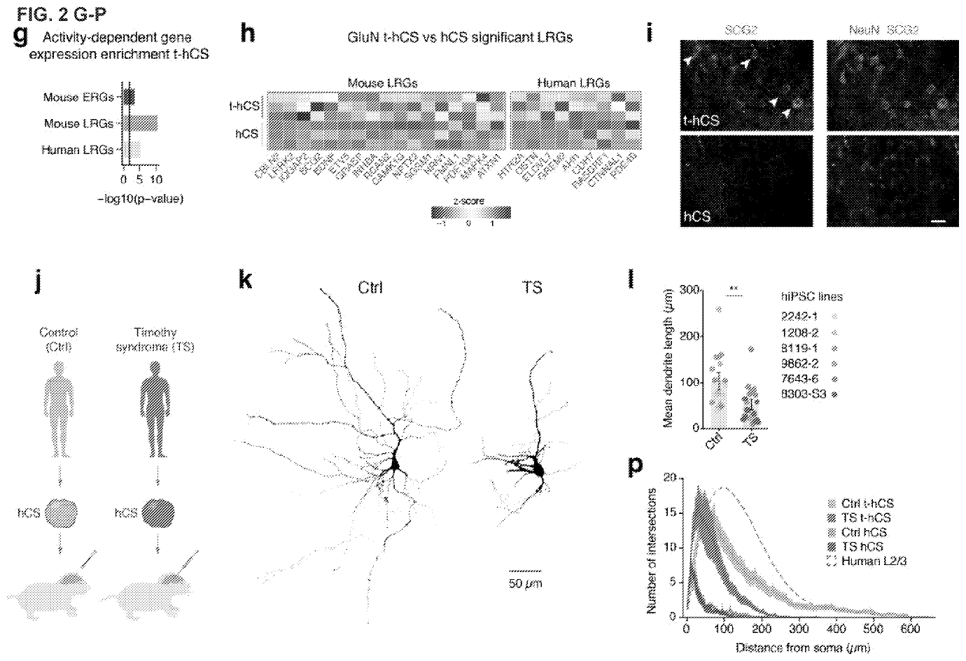

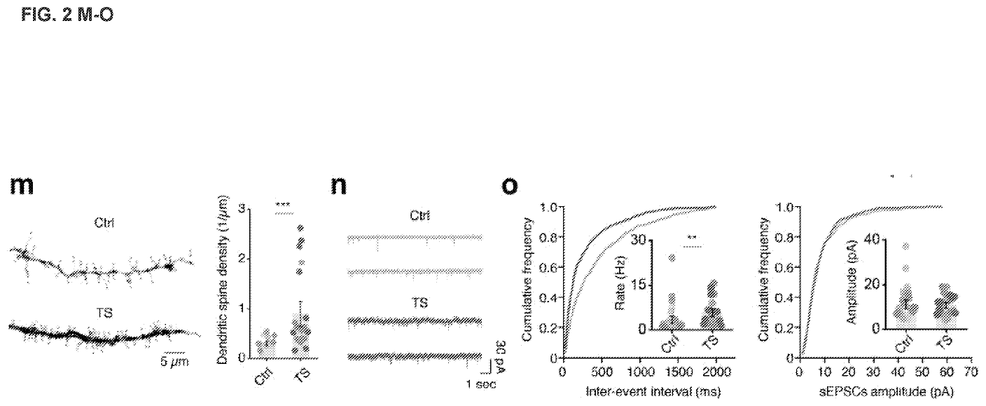

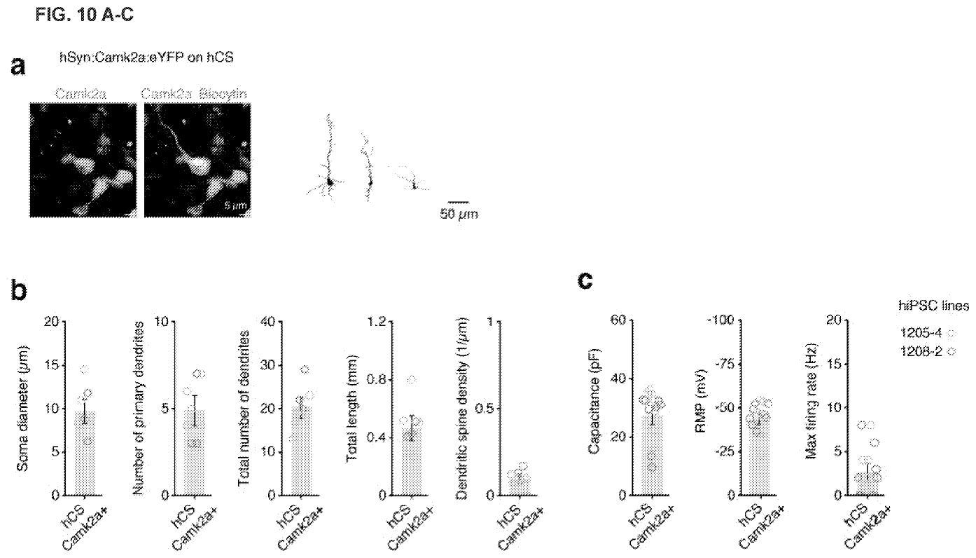

To examine whether these transcriptional changes in t-hCS are associated with morphological differences between in vitro hCS and in vivo t-hCS, biocytin-filled hCS and t-hCS neurons were reconstructed and traced stage-matched in acute slices at 7-8 months of differentiation (FIG. 2a, FIG. 10a). It was discovered that t-hCS neurons were considerably larger with 1.5-fold larger soma diameter, had 2-fold more dendrites and, overall, exhibited a 6-fold increase in total dendrite length compared to hCS in vitro (FIG. 2b, ** P<0.01, *P<0.05, *** P<0.001, FIG. 10b). Moreover, it was observed that significantly higher dendritic spine density in t-hCS neurons (FIG. 2c, ** P<0.01, FIG. 10b). This suggests that t-hCS neurons undergo extensive dendritic extension and arborization which, which in combination with ongoing cellular proliferation likely contributes to the extensive growth of t-hCS following transplantation (FIG. 1d, FIG. 5f) and prompted us to investigate intrinsic and synaptic membrane properties. It was found that the membrane capacitance of t-hCS neurons in vivo was 8-fold higher than observed in vitro (FIG. 8a, *** P<0.001, FIG. 10c), which likely reflects the increase in dendritic length and total number of dendritic spines. The resting membrane potential of t-hCS neurons was more hyperpolarized (by ˜20 mV) compared to in vitro hCS, and current injections elicited significantly higher maximal firing rates (FIG. 2d, e, *** P<0.001, FIG. 10c), consistent with the large and complex morphological features of the cells. Furthermore, the rate of spontaneous excitatory post synaptic current events (sEPSCs) in t-hCS neurons was significantly higher (FIG. 2f, *** P<0.001), indicating that the increase in dendritic spine density observed in t-hCS neurons is associated with an increase in the number of functional excitatory synapses.

In accord with the increased activity observed in t-hCS in ex vivo slices, snRNA-seq revealed an upregulation of activity-dependent gene transcripts in t-hCS compared to stage-matched hCS in vitro. Indeed, t-hCS glutamatergic neurons expressed higher levels of late-response activity-regulated genes (FIG. 2g, h, *** P<0.001) found in prior studies of mouse and cultured human neurons (Hrvatin, S. et al. Nat Neurosci. 2018 January; 21 (1): 120-129; Ataman, B. et al. Nature. 2016 Nov. 10; 539 (7628): 242-247). For example, BDNF, a well characterized activity-regulated factor (Hong, E. J. et al. Neuron. 2008 Nov. 26; 60 (4): 610-24), SCG2 and OSTN, a primate-specific activity-regulated gene (Ataman, B. et al. Nature. 2016 Nov. 10; 539 (7628): 242-247), showed increased expression in t-hCS neurons compared to hCS neurons (FIG. 2g, h). Broad protein expression of the activity-dependent factor SCG2 was confirmed in t-hCS neurons using immunohistochemistry (FIG. 2i). Therefore, across transcriptional, morphological, and functional analyses, t-hCS neurons displayed properties of enhanced maturation compared to hCS neurons.

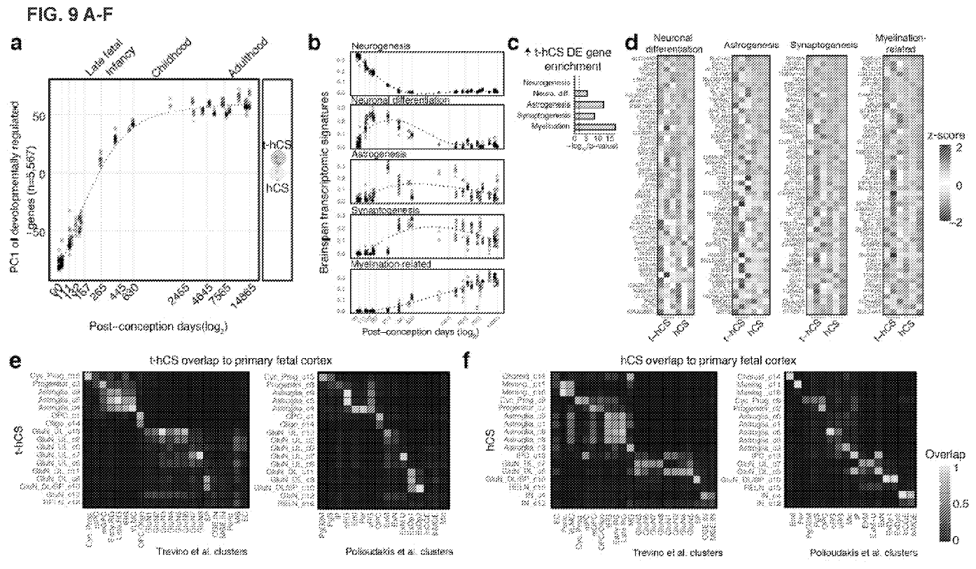

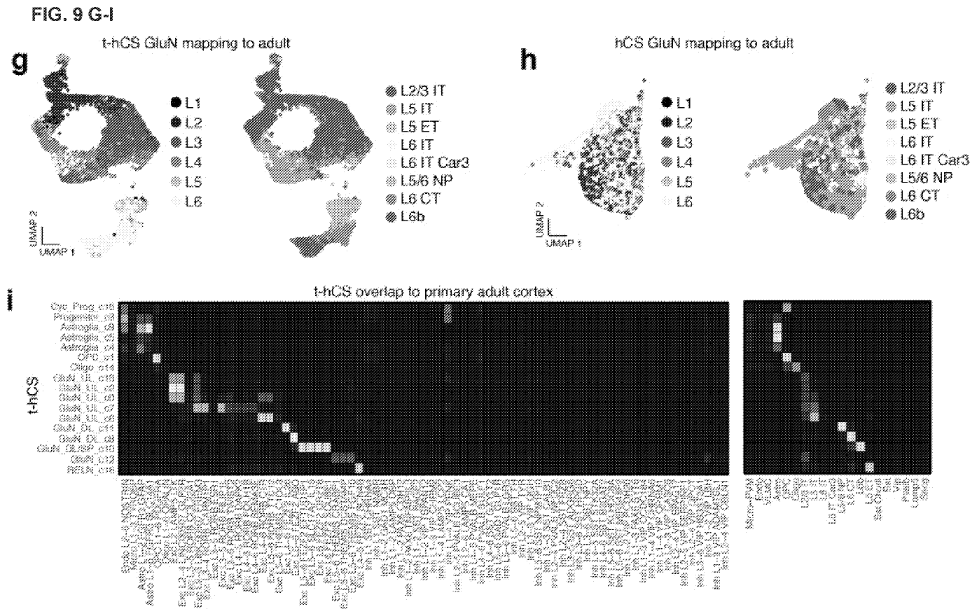

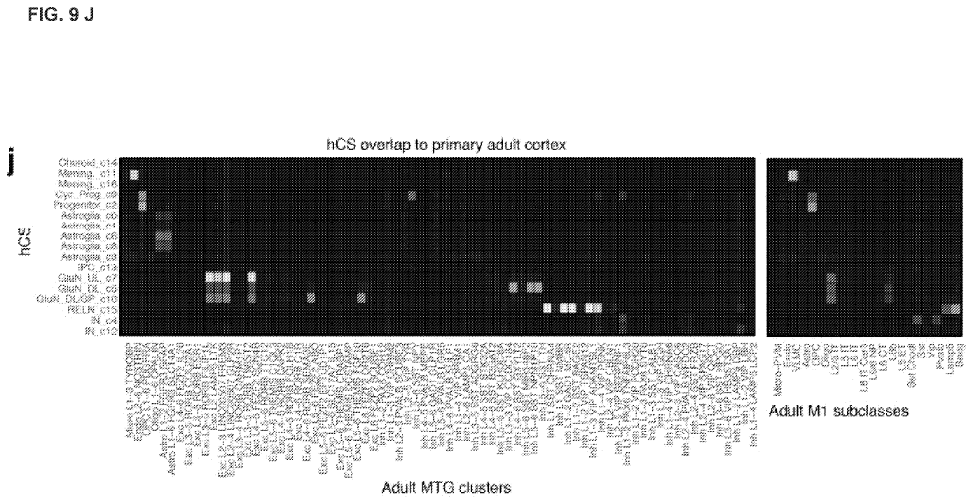

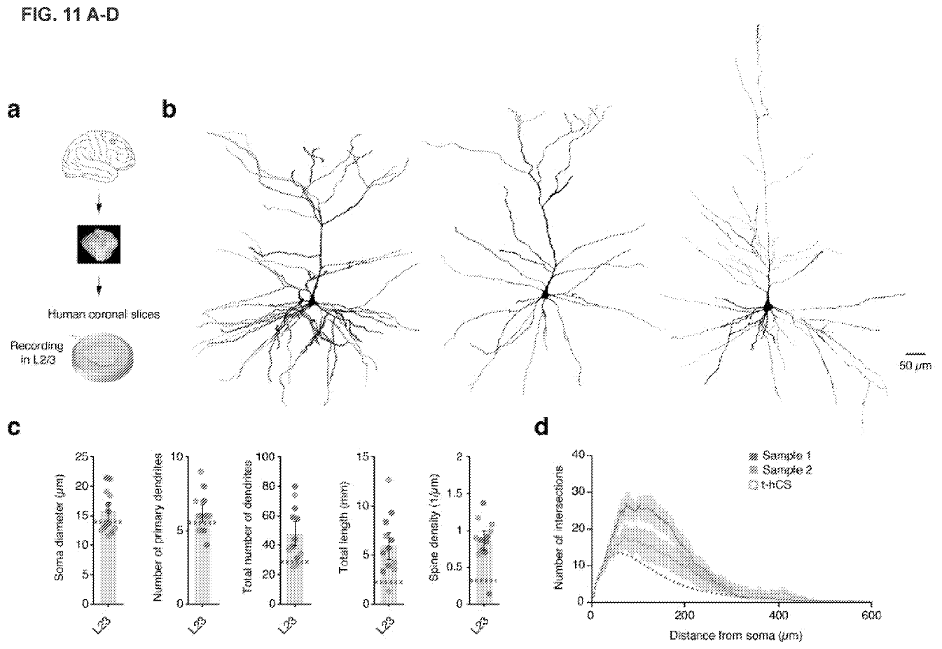

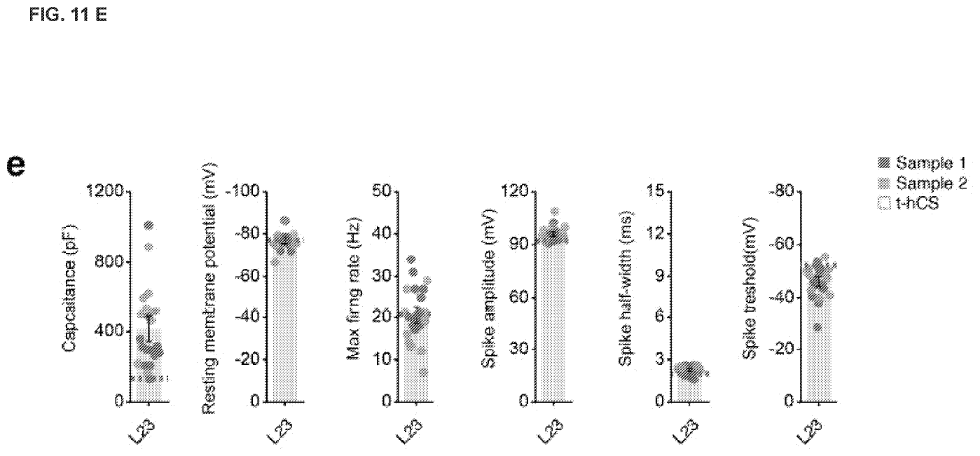

To further assess how t-hCS relates to normal developing human brain, we performed transcriptomic comparisons was performed with human fetal (Polioudakis, D. et al. Neuron. 2019 Sep. 4; 103 (5): 785-801.e8; Trevino, A. E. et al. Cell. 2021 Sep. 16; 184 (19): 5053-5069.e23) and adult cortical cell types (Hodge, R. D. et al. Nature. 2019 September; 573 (7772): 61-68; Bakken, T. E. et al. Nature. 2021 October; 598 (7879): 111-119), as well as developing bulk cortical gene expression data (Zhu, Y. et al. Science. 2018 Dec. 14; 362 (6420): eaat8077) (FIG. 9). Consistent with prior work (Gordon, A. et al. Nat Neurosci. 2021 March; 24 (3): 331-342), the global transcriptomic maturation state of hCS at 7-8 months of differentiation broadly matched in vivo developmental timing and was most equivalent to the late fetal period (FIG. 9). Notably, we observed increased transcriptomic maturation of t-hCS compared to age-matched hCS as well as up-regulation of transcripts associated with synaptogenesis, astrogenesis, and myelination (FIG. 9). At the cell class level, we found evidence for more refined cortical layer subtypes, with t-hCS glutamatergic neuron cluster overlap to adult L2/3, L5, and L6 neuronal subclasses. In contrast, there was more limited cluster overlap between t-hCS glutamatergic neurons and fetal cortical neurons from the 2nd trimester (FIG. 9). To determine whether t-hCS neurons functionally resemble postnatal human neocortical neurons, we performed electrophysiological recordings and anatomical reconstruction of human cortical L2/3 pyramidal neurons in acute ex vivo slices from postnatal human cerebral cortex (FIG. 11a). The electrophysiological properties of L2/3 pyramidal neurons were similar to those of t-hCS pyramidal neurons. Specifically, the resting membrane potential, maximal firing rate, spike amplitude, spike half-width and spike threshold of L2/3 postnatal pyramidal neurons did not differ from those of control t-hCS neurons (FIG. 11e). Morphologically, L2/3 neurons were much more similar to t-hCS than to hCS, although L2/3 cells were slightly longer (1.4 fold), contained more branches overall (1.5 fold) and had higher spine density (3 fold). (FIG. 2p, FIG. 11 b-d).

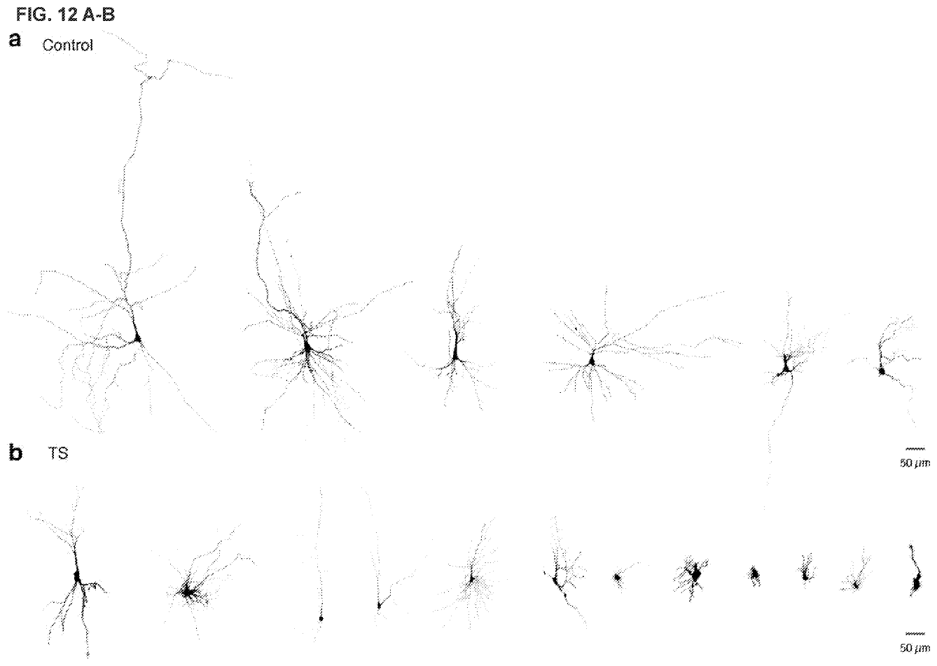

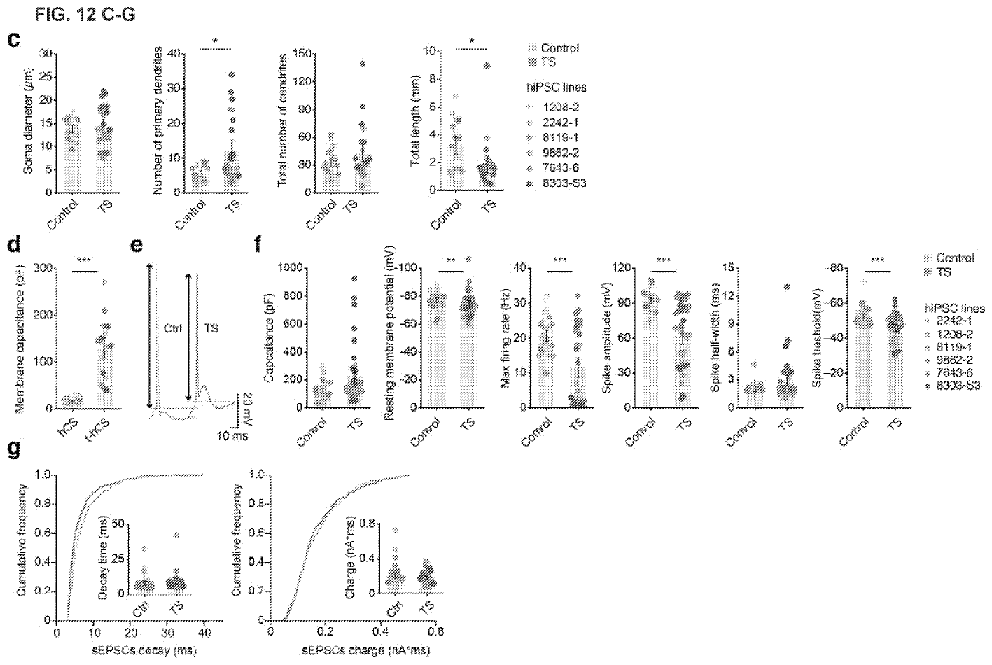

The ability to recapitulate advanced morphological and functional features and activity-dependent transcriptional changes in human cortical neurons prompted us to verify whether t-hCS can be used to uncover disease phenotypes. Timothy syndrome (TS) was focused on—a severe neurodevelopmental disease characterized by autism spectrum disorder and epilepsy. TS is caused by a gain-of-function mutation in the L-type voltage-sensitive calcium channel Cav1.2 (encoded by the CACNA1C gene), which initiates activity-dependent gene transcription in neurons (Ebert, D. H. et al. Nature. 2013 Jan. 17; 493 (7432): 327-37). Generated hCS were generated from hiPSC from three patients with TS that carry the most common substitution (p.G406R) and from three control subjects (FIG. 2j). Following transplantation, investigated neuronal morphology was investigated by filling cells with biocytin and then 3D reconstructing them. It was found that TS neurons had an altered dendritic morphology compared to controls (FIG. 2k, FIG. 12a, b), with a 2-fold increase in the number of primary dendrites and an overall reduction in mean and total dendrite length (FIG. 2l, FIG. 12c, *** P<0.001, *P<0.05, respectively). This was also associated with an increase in synaptic spine density and higher sEPSC frequency in TS compared to control neurons (*** P<0.001), with no significant changes in event amplitude, decay time constant or charge (FIG. 2m-o, FIG. 12d). Interestingly, further analysis revealed an abnormal dendritic branching pattern in TS t-hCS versus control, but not in TS hCS in vitro at a similar differentiation stage (FIG. 2p). This is consistent with an activity-dependent dendritic retraction in TS that was previously reported (Krey, J. F. et al. Nat Neurosci. 2013 February; 16 (2): 201-9) and highlights the ability of this transplantation platform to promote physiologically relevant activity and reveal and validate disease phenotypes in an in vivo context.

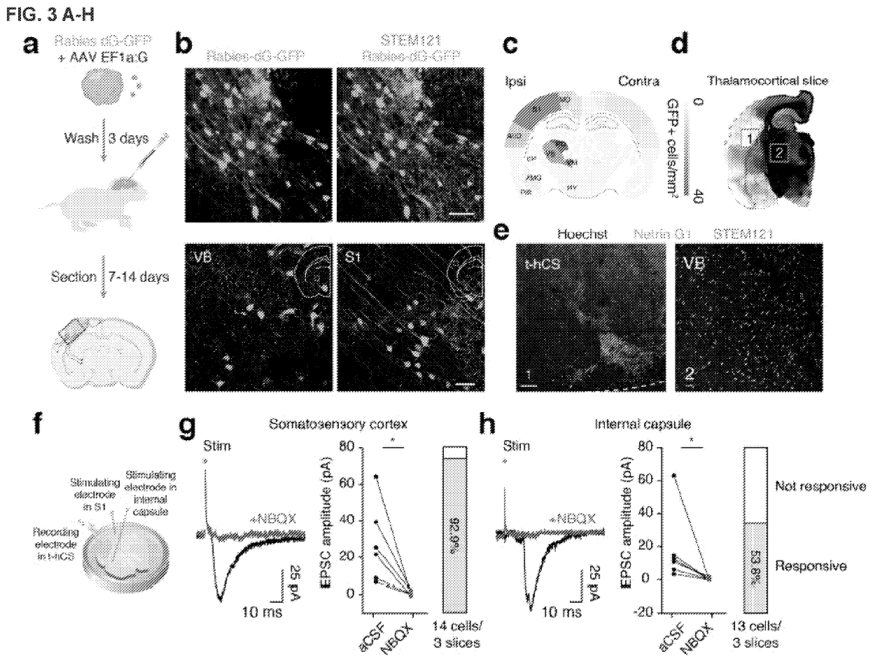

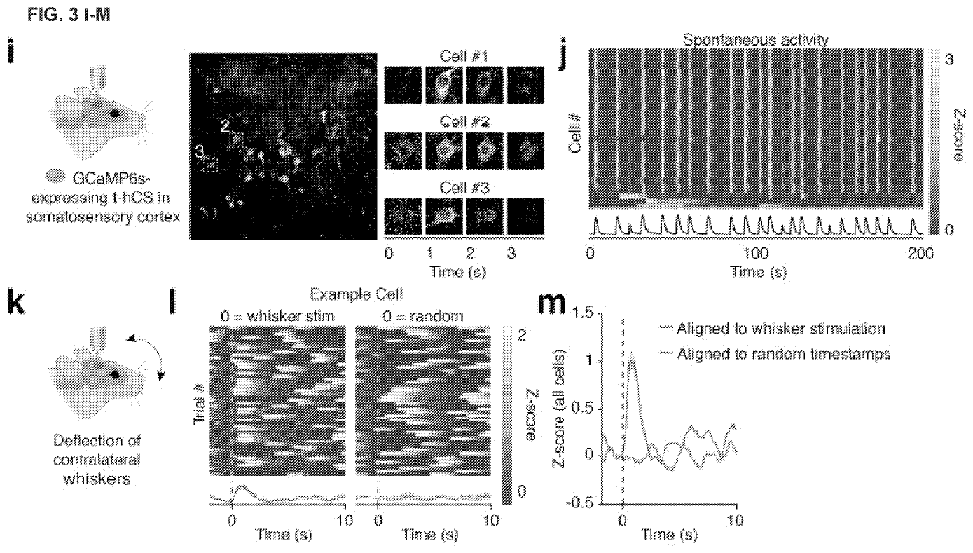

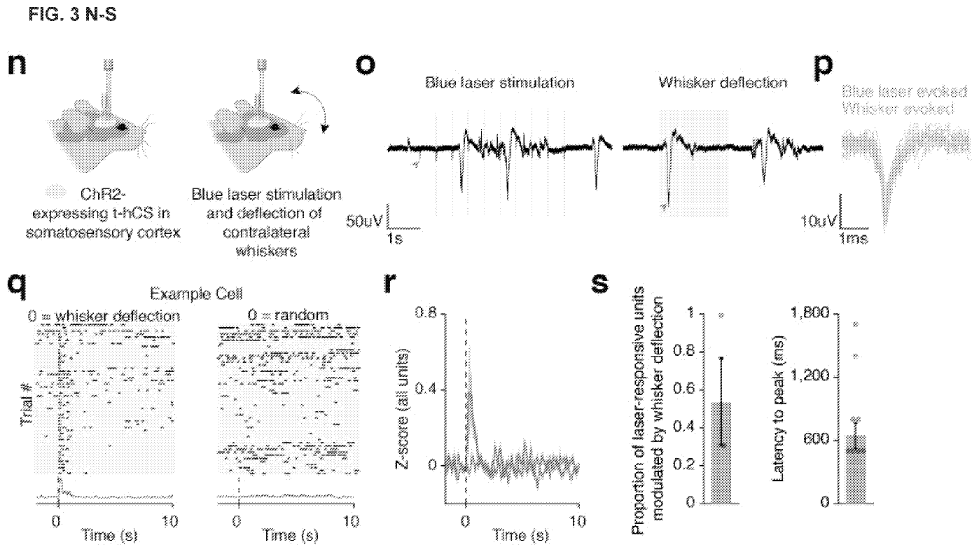

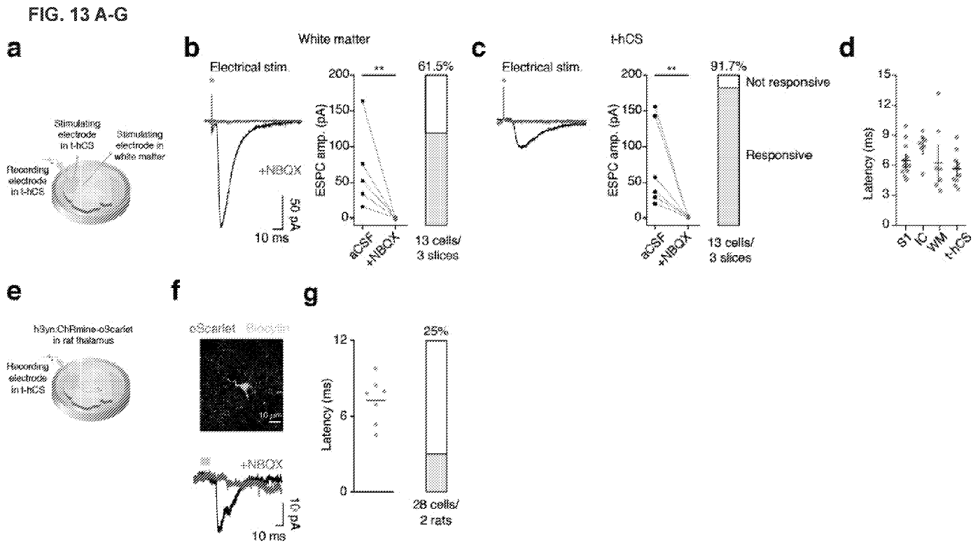

The next question asked was to what extent t-hCS cells functionally integrate into the rat somatosensory cortex. The somatosensory cortex in rodents receives robust synaptic input from the ipsilateral ventrobasal (VB) nucleus and the posterior (PO) nucleus of the thalamus, as well as from the ipsilateral motor and secondary somatosensory cortices and the contralateral primary somatosensory cortex. It was therefore asked whether t-hCS cells received similar patterns of innervation (FIG. 3a). Infected hCS were infected with rabies-dG-GFP/AAV-G and, after 3 days, hCS were transplanted into the rat primary somatosensory cortex. At 7-14 days post-transplantation, dense GFP expression was observed in neurons in the ipsilateral primary somatosensory cortex and the ipsilateral VB of the thalamus (FIG. 3b, c). In addition, antibody staining for the thalamic terminal marker netrin-G1, revealed the presence of thalamic terminals in t-hCS (FIG. 3d-e). These data suggest that t-hCS integrate anatomically into the host somatosensory cortex. To assess whether these afferent projections were capable of evoking synaptic responses in t-hCS cells, performed whole-cell recordings were performed from human cells in acute thalamocortical slices (Agmon, A. et al. Neuroscience. 1991; 41 (2-3): 365-79). Electrical stimulation of the nearby rat somatosensory cortex, thalamocortical fibers of the internal capsule, white matter, or stimulation of t-hCS itself, or optogenetic activation of opsin-expressing thalamic terminals in t-hCS reliably evoked short latency EPSCs in human neurons, which were blocked by application of the AMPA receptor antagonist NBQX (FIG. 3f-h, FIG. 13a-g, *P<0.05). These data demonstrate that t-hCS become anatomically integrated into the rat cerebral cortex and with the rat thalamus, and are capable of being activated by host rat tissue.

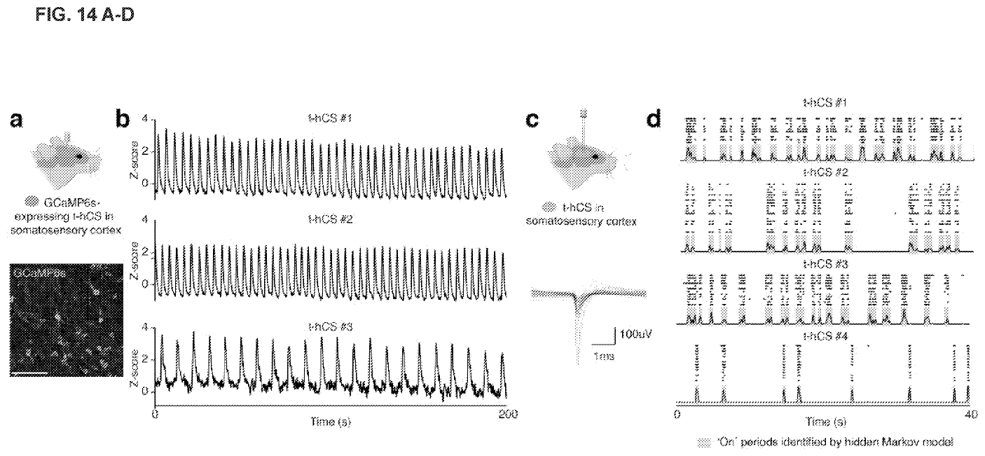

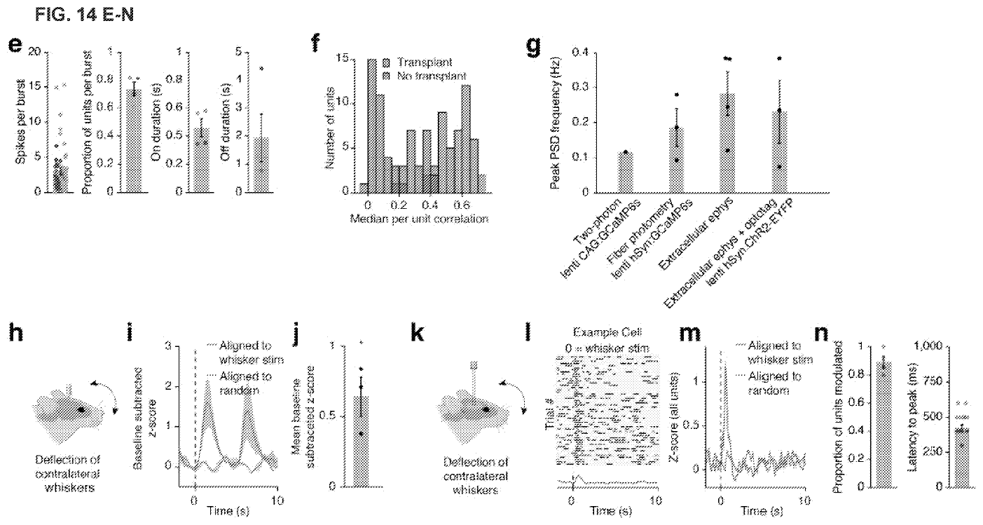

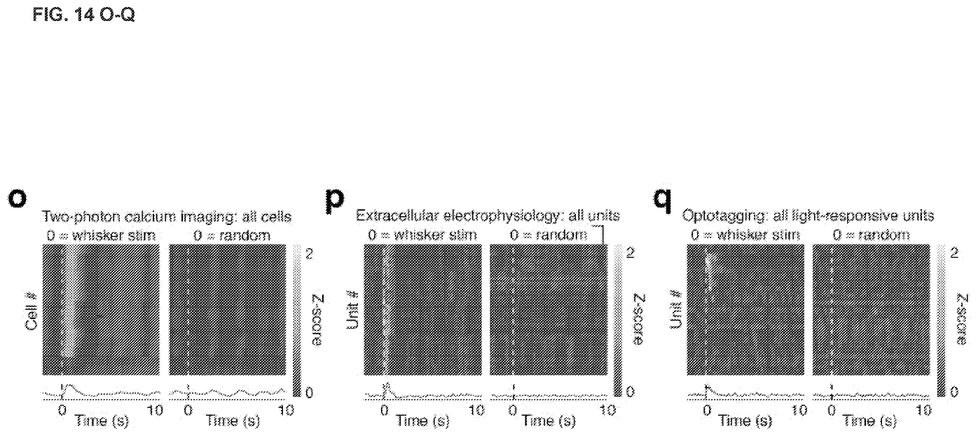

The next question asked was whether t-hCS could be activated by sensory stimuli within an in vivo context. hCS expressing the genetically encoded calcium indicator GCaMP6 s were transplanted into the rat primary somatosensory cortex. After approximately 150 days, conducted two-photon calcium imaging was conducted fiber photometry or in anesthetized animals (FIG. 3i, FIG. 14a). It was found that t-hCS cells exhibited synchronous, rhythmic activity (FIG. 3j, FIG. 14a). To characterize the spiking activity of t-hCS, performed extracellular electrophysiological recordings were performed in anesthetized, transplanted rats (FIG. 14c). To target electrophysiological recordings to the t-hCS region, generated stereotactic coordinates were generated based on images acquired from MRI. Synchronous bursts of activity were observed (as had been observed in t-hCS cells directly identified with two-photon calcium imaging) (FIG. 14d). In addition, 99.4% of NeuN⁺ neurons within t-hCS co-express the human-specific marker HNA; these recorded units thus represent putative human neurons although electrophysiology does not permit species-of-origin identification. These experiments revealed that putative t-hCS neurons exhibited synchronous spiking bursts of ˜460 milliseconds in length that were separated by ˜2-second-long silent periods (FIG. 14 d-e). Individual units fired on average ˜3 spikes per burst, and each burst recruited approximately 73% of recorded units. The activity of individual units was highly correlated, and these correlations were higher than those observed in units identified in non-transplanted animals recorded under the same conditions (FIG. 14f, *** P<0.001). To further characterize the spiking responses of neurons of definitive human origin, opto-tagging experiments was performed in anesthetized rats transplanted with hCS expressing the light-sensitive cation channel, channelrhodopsin (ChR2), where t-hCS neurons were identified by their short latency (<10 ms) response to blue light stimulation. (FIG. 3n-p). t-hCS neurons displayed spontaneous bursts of activity of similar frequency to those observed with both population and single cell calcium imaging, as well as the previous extracellular electrophysiological recordings (FIG. 14g). Spontaneous activity was not observed in age-matched hCS recorded in vitro. To assess whether t-hCS could be activated by sensory stimuli, briefly deflected the rat whiskers were briefly deflected contralateral to the t-hCS (FIG. 3k, n, FIG. 14h, k). In accord with prior studies (Linaro, D. et al. Neuron. 2019 Dec. 4; 104 (5): 972-986.e6), a subset of t-hCS cells displayed increases in calcium activity or increases in spiking activity in response to whisker deflection that were not observed when data were aligned to randomized timestamps (FIG. 3o-r, FIG. 12i, j, 1-q). Indeed approximately 54% of identified opto-tagged single units displayed significant increases in firing rates following whisker stimulation that peaked after ˜650 milliseconds (FIG. 3s). Taken together, these data suggest that t-hCS receive appropriate anatomical inputs, can be activated by electrical stimulation of the rat brain, and can be activated by environmental stimuli.

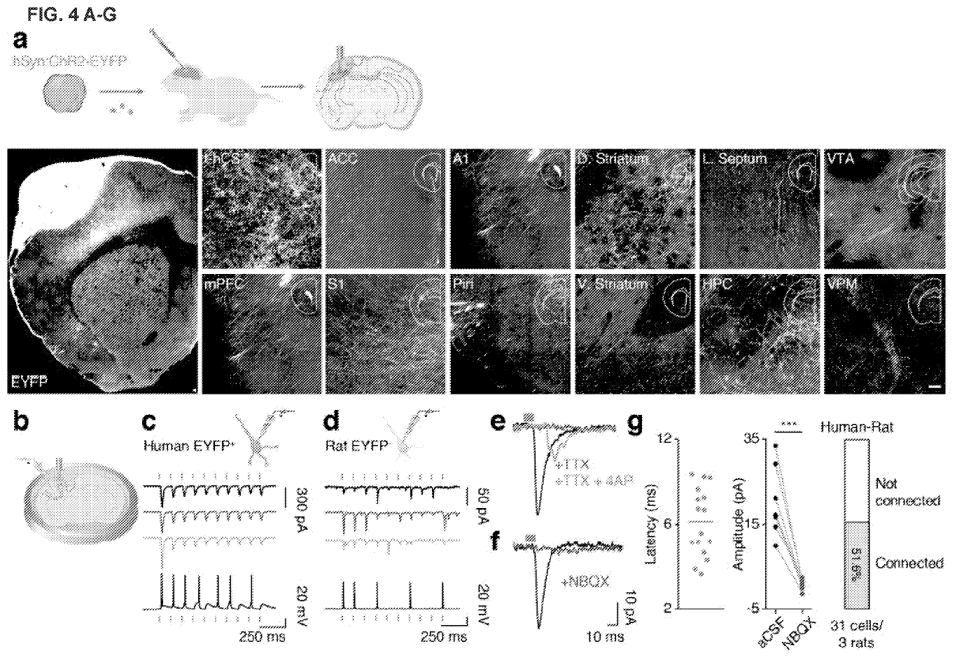

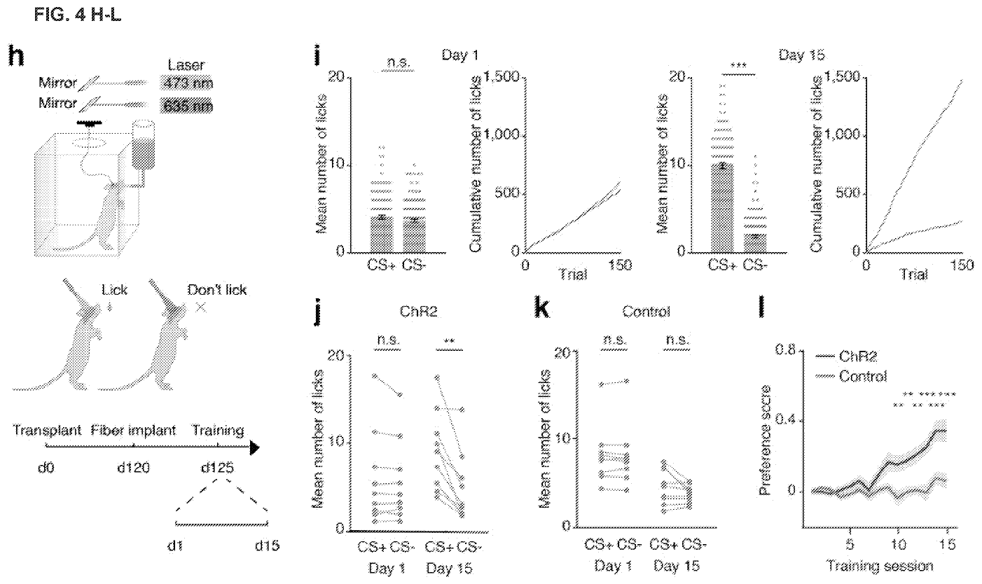

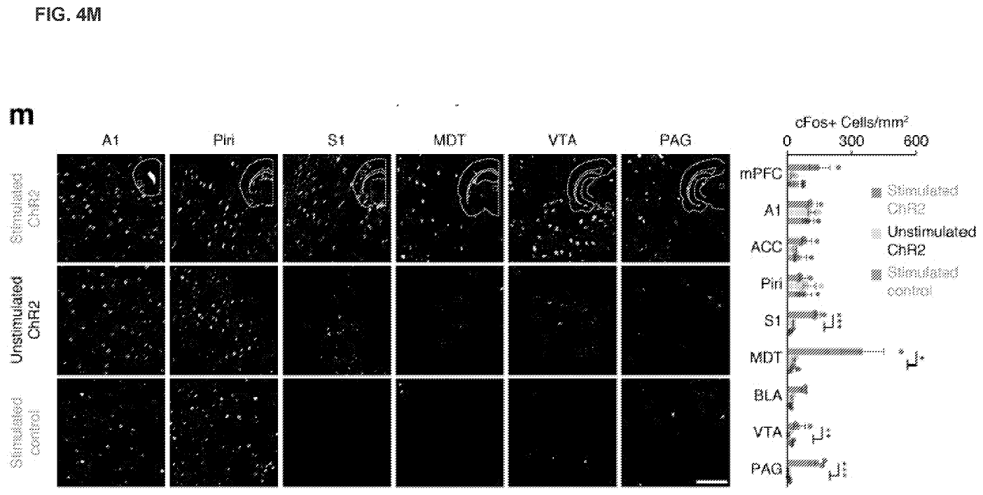

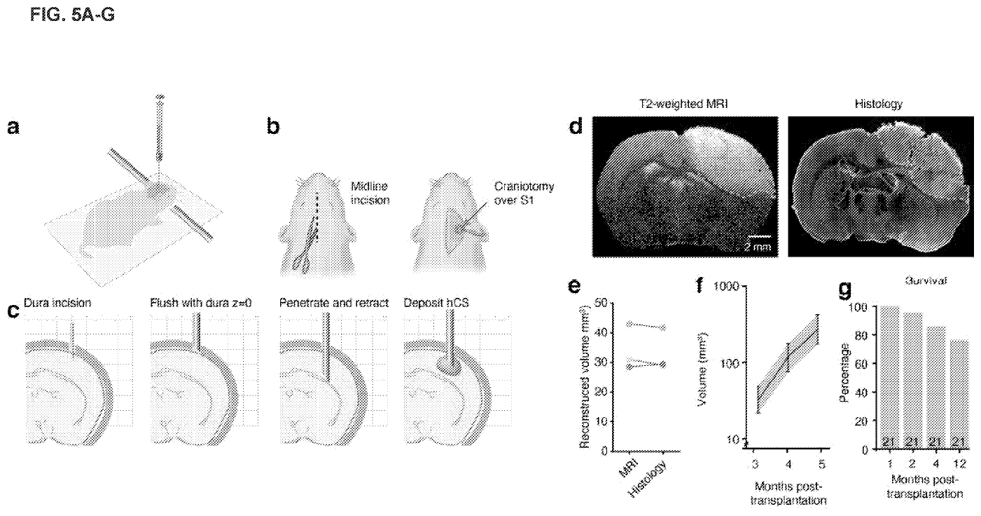

Since t-hCS receive functional inputs from the rat brain, the next question asked was whether t-hCS engage rat circuits and can drive behavior. Whether t-hCS neurons send axonal projections into surrounding rat tissue was first examined. Infected hCS was infected with a lentivirus encoding the light-sensitive cation channel, channelrhodopsin fused to EYFP (ChR2-EYFP). Approximately 110 days later, observed EYFP expression was observed in ipsilateral cortical regions including auditory, motor, and somatosensory cortices, as well as in subcortical regions including the striatum, hippocampus, and thalamus (FIG. 4a). To assess whether these efferent projections were capable of evoking synaptic responses in host rat cells, optically activated ChR2-EYFP-expressing t-hCS cells were optically activated and performed whole-cell recordings from cortical rat cells in acute brain slices. Activation of t-hCS axons with blue light evoked short-latency EPSCs in rat pyramidal cortical neurons, which were blocked by the AMPA receptor antagonist NBQX (FIG. 4b-g, *** P<0.001). Moreover, these responses could be blocked by application of TTX and recovered by 4-AP, indicating that they were evoked by monosynaptic connections (Petreanu, L. et al. Nature. 2009 Feb. 26; 457 (7233): 1142-5) (FIG. 4e).

Finally the question asked was whether t-hCS were capable of modulating rat behavior. To test this, transplanted ChR2-EYFP-expressing hCS were transplanted into the primary somatosensory cortex, and 90 days later implanted an optical fiber into t-hCS for light delivery. Rats were then trained on a modified operant conditioning paradigm (FIG. 4h). Placed animals were placed into a behavioral testing chamber and applied randomly interleaved presentations of 5-second-long blue light (473 nm) and 5 second-long red light (635 nm) laser stimulations. Animals received a water reward if they licked during the blue light stimulation, but not if they licked during the red light stimulation. On the first day of training, animals showed no difference in their licking behavior during either blue or red light stimulation. However, on day 15, animals transplanted with hCS expressing ChR2-EYFP showed increased licking during blue light exposure compared to red light exposure (** P<0.01). Importantly, these changes in licking behavior were not observed in control animals transplanted with hCS expressing a control flurophore (FIG. 4i-l, P>0.05). These data suggest that t-hCS cells can activate rat neurons to drive reward-seeking behaviors. To explore what rat neural circuits might be engaged by t-hCS to drive these changes in behavior, t-hCS were optogenetically activated in trained animals and collected tissue after 90 minutes. Immunohistochemistry revealed expression of the activity-dependent protein c-Fos in several brain regions implicated in motivated behaviors, including mediodorsal thalamus, the ventral tegmental area (VTA) and the periaqueductal grey (PAG), and this was not observed in unstimulated control animals (FIG. 4m, S1 *** P<0.001, MDT *P<0.05, VTA ** P<0.01, PAG *** P<0.001). Taken together these data demonstrate that t-hCS can modulate the activity of rat neurons to drive behavior.

### DISCUSSION

Neural organoids represent a promising system to explore aspects of human development and disease in vitro, but they are limited by the lack of circuit connectivity that exists in vivo. A novel platform was developed in which transplanted hCS were transplanted into the somatosensory cortex of early-postnatal immunocompromised rats to examine human cell development and function in vivo. It was demonstrated that t-hCS develop mature cell types that are not seen in vitro, and that t-hCS integrate both anatomically and functionally into the rodent brain.

The platform described has several advantages in comparison to previous studies that have transplanted human cells into the rodent brain (Espuny-Camacho, I. et al. Neuron. 2013 Feb. 6; 77 (3): 440-56; Linaro, D. et al. Neuron. 2019 Dec. 4; 104 (5): 972-986.e6; Mansour, A. A. et al. Nat Biotechnol. 2018 June; 36 (5): 432-441; Real, R. et al. Science. 2018 Nov. 16; 362 (6416): eaau1810; Kitahara, T. et al. Stem Cell Reports. 2020 Aug. 11; 15 (2): 467-481; Xiong, M. et al. Cell Stem Cell. 2021 Jan. 7; 28 (1): 112-126.e6). First, prior studies have typically been performed in adult animals, where neural circuits are well-established and largely static. By contrast, hCS were transplanted into the developing cerebral cortex of early-postnatal rats, which likely favors anatomical and functional integration. Second, MRI monitoring of t-hCS allowed the examination of graft position and growth in living animals, enabling us to perform long-term studies across multiple animals and establish reliability across multiple hiPSC lines. Finally, several prior studies have transplanted human-derived neurons as dissociated cells and after exposure to small molecules that halt corticogenesis (Linaro, D. et al. Neuron. 2019 Dec. 4; 104 (5): 972-986.e6). Here, self-organizing 3D neural cultures that undergo corticogenesis were transplanted. Engrafting intact organoids, rather than a dissociated single cell suspension, is less disruptive to human cells and likely facilitates integration and the generation of a unit of human cortical neural cells in the rat brain. This enables the implementation of a series of assays to examine how these cells interact with both host rat circuits and other human cells

The advances of this platform enabled us to achieve high level circuit integration of t-hCS, which receives functional sensory input and can drive behavioral responses. This allowed us to uncover a human activity-dependent transcriptional program and revealed that t-hCS neurons undergo advanced morphological and functional maturation that are not seen in vitro and may more closely resemble developmental patterns that are observed in vivo. For instance, the shift to a more hyperpolarized resting membrane potential in t-hCS is similar to studies in slices of human cortical tissue or in vivo recordings (Kalmbach, B. E. et al. Neuron. 2018 Dec. 5; 100 (5): 1194-1208.e5). The molecular programs that underlie the dramatic differences that was observed between in vivo t-hCS and in vitro hCS remain to be discovered, but this feature enabled us to identify disease, activity-dependent phenotypes that could not be detected in vitro. Finally, the integration of t-hCS into rodent neural circuits allowed us to establish links between the activity of human cells and animal behavior. Activation of t-hCS neurons was able to drive learned reward-seeking behavior. While the locus of plasticity underlying this learning remains to be established, whether at human-rat, human-human, or rat-rat synapses, these data do demonstrate for the first time that t-hCS neurons can modulate rat neuron activity to drive behavioral responses.

Nonetheless, that despite the advances this platform offers, there are temporo-spatial and cross-species limitations that preclude the formation of human neural circuits with high fidelity even after transplantation at early stages in development. For example, it is currently unclear whether the spontaneous activity observed in t-hCS represents a developmental phenotype, similar to rhythmic activity observed during both rodent and human fetal cortical development (Molnar, Z. et al., Science. 2020 Oct. 16; 370 (6514): eabb2153), or related to the lack of inhibitory cell types present in t-hCS. Future work will be aimed at incorporating other cell types such as human microglia, human endothelial cells, and various proportions of GABAergic interneurons, as has been shown in vitro using assembloids (Birey, F. et al. Nature. 2017 May 4; 545 (7652): 54-59), as well as understanding how neural integration and processing might be altered in patient-derived t-hCS at the transcriptional, synaptic, and behavioral levels.

Overall, this in vivo platform represents a powerful resource to complement in vitro studies of human brain development and disease. This platform will allow the ability to uncover new circuit-level phenotypes in patient-derived cells that have otherwise been elusive.

### Methods

hCS generation. hCS were generated from hiPSC as previously described (Pasca, A. M. et al. Nat Methods. 2015 July; 12 (7): 671-8; Yoon, S. J. et al. Nat Methods. 2019 January; 16 (1): 75-78). To initiate the generation of hCS from hiPSC cultured on feeders, intact hiPSC colonies were lifted from the plates using dispase (0.35 mg ml⁻¹) and transferred to ultra-low-attachment plastic dishes (Corning) in hiPSC medium supplemented with the two SMAD inhibitors dorsomorphin (5 μM, Sigma-Aldrich, P5499) and SB-431542 (10 μM, Tocris, 1614) and the ROCK inhibitor Y-27632 (10 μM, Selleckchem, S1049). For the first five days, the hiPSC medium was changed every day and supplemented with dorsomorphin and SB-431542. On the sixth day in suspension, neural spheroids were transferred to neural medium containing neurobasal-A (Life Technologies, 10888), β-27 supplement without vitamin A (Life Technologies, 12587), GlutaMax (1:100, Life Technologies), penicillin and streptomycin (1:100, Life Technologies) and supplemented with the epidermal growth factor (EGF, 20 ng ml⁻¹; R&D Systems) and fibroblast growth factor 2 (FGF2, 20 ng ml⁻¹; R&D Systems) until day 24. From day 25 to day 42, the medium was supplemented with brain-derived neurotrophic factor (BDNF, 20 ng ml⁻¹, Peprotech) and neurotrophin 3 (NT3, 20 ng ml⁻¹, Peprotech) with medium changes every other day. From day 43 onward, hCS were maintained in unsupplemented neurobasal-A medium (NM, Thermo Fisher, 1088022) with medium changes every 4-6 days. For the generation of hCS from hiPSC cultured on feeder-free conditions, hiPSC were incubated with accutase (Innovate Cell Technologies, AT-104) at 37° C. for 7 min, dissociated into single cells, and seeded into AggreWell 800 plates (STEMCELL Technologies, 34815) at a density of 3×10⁶ single cells per well in Essential 8 medium supplemented with the ROCK inhibitor Y-27632 (10 μM, Selleckchem, S1049). After 24 h, spheroids were collected from each microwell by pipetting medium in the well up and down and transferring it into ultra-low-attachment plastic dishes (Corning, 3262) containing Essential 6 medium (Life Technologies, A1516401) supplemented with dorsomorphin (2.5 μM, Sigma-Aldrich, P5499) and SB-431542 (10 μM, Tocris, 1614). From day 2 to day 6, Essential 6 medium was changed every day and supplemented with dorsomorphin and SB-431542. From the sixth day in suspension, neural spheroids were transferred to neurobasal medium and maintained as described above.

Transplantation into athymic newborn rats. All animal procedures followed animal care guidelines approved by Stanford University's Administrative Panel on Laboratory Animal Care (APLAC). Pregnant RNU euthymic (mu/+) rats were purchased (Charles River Laboratories, Wilmington, MA) or bred in house. Animals were maintained under a 12-h light/dark cycle and provided food and water ad libitum. Three to seven day-old athymic (FOXN1^{-/-}) rat pups were identified by an immature whisker growth before culling. Pups were anesthetized with 2-3% isoflurane and mounted on a stereotaxic frame. A craniotomy, at about 2-3 mm in diameter was performed above S1, preserving the dura intact. Next, the dura mater was punctured using a 30 G needle (˜0.3 mm) close to the lateral side of the craniotomy. A hCS was next moved onto a thin 3×3 cm parafilm and excess media was removed. Using a Hamilton syringe connected to a 23 G, 45 degrees needle, the hCS was gently pulled into the most distal tip of the needle. The syringe was next mounted on a syringe pump connected to the stereotaxic device. The sharp tip of the needle was next positioned above the 0.3 mm-wide pre-made puncture in the dura mater (z=0 mm), the syringe was reduced 1-2 mm (z=˜-1.5 mm), and until a tight seal between the needle and the dura mater was formed. Next, the syringe was elevated to the center of the cortical surface at z=−0.5 mm, and the hCS was ejected at a speed of 1-2 μL a minute. After hCS injection was completed, the needle was retracted at a rate of 0.2-0.5 mm per minute, the skin was closed, and the pups were immediately placed on a warmed heat pad until complete recovery. Some animals were engrafted bilaterally.

Magnetic Resonance Imaging of transplanted rats. All animal procedures followed animal care guidelines approved by Stanford University's Administrative Panel on Laboratory Animal Care (APLAC). Rats (>60 days post-transplantation) were anesthetized with 5% isoflurane for induction and 1-3% isoflurane during imaging. For imaging, an actively-shielded Bruker 7 Tesla horizontal bore scanner (Bruker Corp., Billerica, MA, USA) with International Electric Company (IECO) gradient drivers, a 120 mm inner diameter (ID) shielded gradient insert (600 mT/m, 1,000 T/m/s), AVANCE III electronics, eight-channel multi-coil RF and multinuclear capabilities, and the supporting Paravision 6.0.1 platform were used. Acquisitions were performed with an 86 mm ID actively de-couplable volume radiofrequency (RF) coil with a four-channel cryo-cooled receive-only RF coil. Finally, 3D volume rendering and analysis were performed using Imaris (BitPlane) built-in surface estimation functions. Axial 2D Turbo-RARE (TR=2500 ms, TE=33 ms, 2 averages) 16 slice acquisitions were performed with 0.6-0.8 mm slice thickness, with 256×256 samples. Signal was received with a 2 cm inner-diameter quadrature transmit-receive volume radiofrequency coil (Rapid MR International, LLC.). Successful transplantations were defined as transplantations that resulted in a continuous area of T2-weighted MRI signal in the transplanted hemisphere. Failed transplantations were defined as transplantations that did not result in a continuous area of T2-weighted MRI signal in the transplanted hemisphere. Subcortical t-hCS were excluded from subsequent analyses.

Lentivirus labeling and G-deleted rabies infections. To stably express GCaMP6 s in hCS for two-photon calcium imaging, hiPSC were infected with pLV [Exp]-EF1a::GcaMP6 s-Puro followed by antibiotic selection. Briefly, the cells were dissociated with EDTA and suspended at a density of ˜300,000 cells in 1 mL in E8 in the presence of polybrene (5 μg/mL) and 15 μL of virus. Cells were then incubated in suspension for 60 min and plated at a density of 50,000 cells/well. Once confluent, cells were treated with 5-10 μg/mL puromycin for 5-10 days or until stable colonies appeared. Acute infections of hCS were performed as previously described (Yoon, S. J. et al. Nat Methods. 2019 January; 16 (1): 75-78) with a few modifications. In brief, day 30-45 hCS were transferred to 1.5 ml microcentrifuge Eppendorf tubes containing 100 μL neural medium. Next, ˜90 μL of medium was removed and 3-6 μl of high-titer lentivirus (0.5×10⁸-1.2×10⁹) was added to the tube, and the hCS were moved to the incubator for 30 min. Next, 90-100 μL medium were added to each tube and the tubes were returned to the incubator overnight. The next day, hCS were transferred to fresh neural medium in low-attachment plates. After 7 days, hCS were moved to glass-bottom 24-well plates for imaging and infection quality assessment. pLV [Exp]-SYN1::EYFP and pLV [Exp]-SYN1::ChR2 were generated by VectorBuilder. Lentivirus was used for most experiments as it is incorporated into the host genome permitting reporter expression in the infected cell lineage. For rabies tracing, day 30-45 hCS were coinfected with rabies-ΔG-eGFP and AAV-DJ-EF1a-CVS-G-WPRE-pGHpA (Addgene, Plasmid #67528), thoroughly washed over the course of 3 days, transplanted into the rat S1 and maintained in vivo for 7-14 days.

Tissue preparation and immunohistochemistry. For immunocytochemistry, animals were anesthetized and transcardially perfused with PBS followed by 4% paraformaldehyde (PFA in PBS, Electron Microscopy Sciences). Brains were post-fixed with 4% PFA for either 2 h or overnight at 4° C., cryopreserved in 30% sucrose in PBS for 48-72 hours, embedded in 1:1, 30% sucrose: OCT (Tissue-Tek OCT Compound 4583, Sakura Finetek), and sectioned coronally at 30 μm using a cryostat (Leica). For immunohistochemistry in thick sections, animals were perfused with PBS, brains were then dissected and sectioned coronally at 300-400 μm using a vibratome (Leica), and sections then fixed with 4% PFA for 30 min. Cryosections or thick sections were then washed with PBS, blocked for 1 h at room temperature (10% normal donkey serum (NDS), 0.3% Triton X-100 diluted in PBS), and incubated at 4° C. with primary antibodies in blocking solution. Cryosections were incubated overnight while thick sections were incubated for 5 days. Primary antibodies used were: anti-BRN2 (Mouse, 1:500, Millipore, MABD51), anti-CTIP2 (Rat, 1:300, Abcam, ab18465), anti-GFAP (Rabbit, 1:1,000, Dako, Z0334), anti-GFP (Chicken, 1:1,000, GeneTex, GTX13970), anti-human nuclear antigen (HNA, Mouse, 1:200, Abcam, ab191181), anti-NeuN (Rabbit, 1:500, Millipore ABN78), anti-PDGFRA (Rabbit, 1:200, Santa Cruz, sc-338), anti-PPP1R17 (Rabbit, 1:200, Atlas Antibodies, HPA047819), anti-RECA-1 (Mouse, 1:50, Abcam, ab9774), anti-SCG2 (Rabbit, 1:100, Proteintech, 20357-1-AP), anti-SOX9 (Goat, 1:500, R&D Systems, AF3075), NetrinG1a (Goat, 1:100, R&D Systems, AF1166) and anti-STEM121 (Mouse, 1:200, Takara Bio, Y40410), anti-CTIP2 (Rat, 1:300, Abcam, an18465), anti-SATB2 (Mouse, 1:50, Abcam, ab51502), Anti-GAD 65/67 (Rabbit, 1:400, Milipore, ABN904). Sections were then washed with PBS and incubated with secondary antibodies for either 1 h at RT (cryosections) or overnight at 4° C. (thick sections). Alexa Fluor secondary antibodies (Life Technologies) diluted in blocking solution at 1:1,000 were used. Following washes with PBS, nuclei were visualized with Hoechst 33258 (Life Technologies). Finally, slides were mounted for microscopy with cover glasses (Fisher Scientific) using Aquamount (Polysciences) and imaged on a Keyence fluorescence microscope or Leica TCS SP8 confocal microscope. Images were processed in ImageJ (Fiji). To quantify the fraction of human neurons in t-hCS and rat cortex, 387.5 μm-wide rectangular images were taken at the t-hCS center, edge or from the adjacent rat cortex. The edge of the graft was determined by assessing changes in tissue transparency, the presence of HNA⁺ nuclei and/or tissue autofluorescence. In each image, the total number of NeuN⁺ and HNA⁺ cells was divided by the total number of Hoechst⁺ and NeuN⁺ cells within the same area. Two images, taken at least 1 mm apart, were averaged to reduce statistical error.

Single nuclei dissociation and gene expression. One week prior to sample collection, animals transplanted with hCS (˜8 months of differentiation) were housed in a dark room and whiskers were trimmed to minimize sensory stimulation. Nuclei isolation was performed as described in (Matson, K. J. E. et al. J Vis Exp. 2018 Oct. 12; (140): 58413) with some modifications. Briefly, t-hCS and hCS were disrupted using the detergent-mechanical cell lysis method with a 2-ml glass tissue grinder (Sigma-Aldrich/KIMBLE, D8938). Crude nuclei were then filtered using a 40 μm filter and centrifuged at 320×g for 10 minutes at 4° C. before performing a sucrose density gradient. After a centrifugation step (320×g, 20 minutes at 4° C.), samples were resuspended in 0.04% BSA/PBS supplemented with 0.2 U/μl RNAse inhibitor (Ambion 40 U/μl, AM2682) and passed through a 40 μm flowmi filter. Dissociated nuclei were then resuspended in PBS containing 0.02% BSA and loaded onto a Chromium Single cell 3′ chip (with an estimated recovery of 8,000 cells per channel). snRNA-seq libraries were prepared with the Chromium Single cell 3′ GEM, Library & Gel Bead Kit v3 (10× Genomics). Libraries from different samples were pooled and sequenced by Admera Health on a NovaSeq S4 (Illumina).

Singe nuclei expression analysis. Gene expression levels were quantified for each putative nuclei barcode using the 10× Genomics CellRanger analysis software suite (version 6.1.2). Specifically, reads were mapped to a combined human (GRCh38, Ensemble release 98) and rat (Rnor_6.0, Ensemble release 100) reference genome created using the mkref command and quantified using the count command with -include-introns=TRUE to include reads mapping to intronic regions. For t-hCS samples, human nuclei were identified based on a conservative requirement of at least 95% of total mapped reads aligning to the human genome. All subsequent analyses were performed on the filtered barcode matrices outputted from CellRanger using the R (version 4.0.2) package Seurat (version 4.0.4) (Stuart, T. et al. Cell. 2019 Jun. 13; 177 (7): 1888-1902.e21)

To ensure only high-quality nuclei were included for downstream analyses a two-step filtering process was implemented for each sample. First, low quality nuclei with less than 1,000 unique genes detected and with mitochondrial counts accounting for greater than 20% of the total counts were identified and removed. Subsequently, raw gene count matrices were normalized using the sctransform function, which also identified the top 3,000 highly variable genes using default parameters. Dimensionality reduction using principal component analysis (PCA) on the top variable genes was performed and clusters of nuclei were identified in PCA space by shared nearest-neighbor graph construction and modularity detection implemented by the FindNeighbors and FindClusters functions using a dataset dimension of 30 with default parameters. An initial round of clustering was performed with a high-resolution parameter (resolution=2) to identify subpopulations of low-quality cells based on outlier low gene count detection, predominantly mitochondrial marker genes, and/or high proportions of putative doublets using the DoubletFinder package (McGinnis, C. S. et al. Cell Syst. 2019 Apr. 24; 8 (4): 329-337.e4). Marker genes for each cluster were determined using the FindMarkers function with default parameters. Lastly, clusters of non-ectodermal lineage nuclei (based on expression of DCN, BGN, FOXC2) were removed from the hCS sample and the t-hCS samples.

Following low quality nuclei removal, each sample was reanalyzed using the above Seurat workflow with a clustering resolution=0.5 and embedded for visualization purposes with Uniform Manifold Approximation and Projection (UMAP) (Becht, E. et al. Nat Biotechnol. 2018 Dec. 3). The two t-hCS samples were combined using the IntegrateData function with above parameters. For the integrated t-hCS dataset log normalized count data using the NormalizeData function was used for further downstream analyses. Major cell classes were identified and classified through marker gene expression and comparisons with published literature (Nowakowski, T. J. et al. Science. 2017 Dec. 8; 358 (6368): 1318-1323; Trevino, A. E. et al. Cell. 2021 Sep. 16; 184 (19): 5053-5069.e23; Hodge, R. D. et al. Nature. 2019 September; 573 (7772): 61-68; Polioudakis, D. et al. Neuron. 2019 Sep. 4; 103 (5): 785-801.e8). Specifically, glutamatergic neurons were identified by the expression of SLC17A7 and SNAP25. Deep and superficial layer neuron were classified based on previously identified layer specific marker expression. Interneurons were determined by the presence of GAD1/GAD2. Oligodendrocyte progenitor cells had high expression of PDGFRA, oligodendrocytes expressed markers of myelination (MOG, MBP, MYRF), and astrocytes were identified by high expression of AQP4.

Differential gene expression of glutamatergic neurons between t-hCS and hCS were performed using the FindMarkers function on count data normalized using the NormalizeData function. Gene Ontology (GO) enrichment analyses among significant genes (adjusted p-values less than 0.05) with a fold change increase of at least 2 were performed using the ToppGene (https://toppgene.cchmc.org/) suite (Chen, J. et al. Nucleic Acids Res. 2009 July; 37 (Web Server issue): W305-11). The ToppFun application was used with default parameters and reported Benjamini-Hochberg-adjusted P values calculated from hypergeometric tests from GO annotations.

To explore activity-dependent transcriptional markers, obtained gene lists were obtained from prior published studies. Specifically, significantly up-regulated late-response genes in glutamatergic neurons identified by snRNA-seq collection of mouse visual cortex 4 hours after visual stimulation were obtained from Table S3 from Hrvatin, S. et al. Nat Neurosci. 2018 January; 21 (1): 120-129. Similarly, human-enriched late-response genes were obtained from human fetal brain cultures activated with KCl and collected 6 hours after stimulation, which were filtered for genes significantly up-regulated in humans but not rodents (Ataman, B. et al. Nature. 2016 Nov. 10; 539 (7628): 242-247) (Table S4). Gene module scores for these gene lists were calculated using the AddModuleScore function in Seurat.

Ex vivo t-hCS slice electrophysiology. Rats were anesthetized with isoflurane and brains were removed and placed in cold (˜4° C.) oxygenated (95% O₂, 5% CO₂) sucrose slicing solution containing (in mM): 234 sucrose, 11 glucose, 26 NaHCO₃, 2.5 KCl, 1.25 NaH₂PO₄, 10 MgSO₄, and 0.5 CaCl₂) (˜310 mOsm). Coronal rat brain slices (300-400 μm), containing t-hCS, were sectioned using a Leica VT1200 vibratome as previously described (Huguenard, J. R. et al. J Neurosci. 1994 September; 14 (9): 5485-502). Slices were then moved to a continuously oxygenated slice chamber at RT, which contained artificial cerebrospinal fluid (aCSF) made with (in mM): 10 glucose, 26 NaHCO₃, 2.5 KCl, 1.25 NaHPO₄, 1 MgSO₄, 2 CaCl₂), and 126 NaCl (298 mOsm) for at least 45 min prior to recording. Slice recordings were performed in a submerged chamber where they were continuously perfused with aCSF (bubbled with 95% O₂ and 5% CO₂). All data were recorded at room temperature. t-hCS Neurons were patched with a borosilicate glass pipette filled with an internal solution containing 127 mM potassium gluconate, 8 mM NaCl, 4 mM magnesium ATP, 0.3 mM sodium GTP, 10 mM HEPES and 0.6 mM EGTA, pH 7.2, adjusted with KOH (290 mOsm). For reconstruction purposes, biocytin (0.2%) was added to the recording solution.

Data were acquired with a MultiClamp 700B Amplifier (Molecular Devices) and a Digidata 1550B Digitizer (Molecular Devices), low pass filtered at 2 kHz, digitized at 20 kHz and analyzed using Clampfit (Molecular Devices) and custom Matlab (Mathworks) functions. The liquid junction potential was calculated using JPCalc, and recordings were corrected with an estimated-14 mV. The I-V manipulations were constructed from a series of current steps in 10-25 pA increments from −250 to 750 pA.

Electrical stimulations of thalamic, white matter, and S1 afferents during patch clamp recordings of hCS neurons were performed in thalamocortical slices, as previously described (Agmon, A. et al. Neuroscience. 1991; 41 (2-3): 365-79). Briefly, the brain was situated on a 3D printed stage tilted at 10 degrees and the frontal brain was cut with a 35 degrees angle. The brain was then glued onto the cut surface and slices which preserve the thalamocortical projection axons were generated. Bipolar tungsten electrodes (0.5 Mohm) were mounted on a second micromanipulator and were strategically positioned to stimulate 4 regions per each cell (internal capsule, white matter, S1 and hCS). Synaptic responses were recorded following a 300 μA phasic stimulation at 0.03-0.1 Hz.

ChR2-expressing hCS neurons were activated with 480 nm, LED-generated (Prizmatix) light pulses delivered via a 40× objective (0.9 NA, Olympus) onto ChR2-expressing processes next to the recorded cell. The illumination field was ˜0.5 mm in diameter at a total intensity of 10-20 mW. Pulse width was set at 10 ms, which corresponds to the pulse delivered during behavioral training experiments. Multiple stimulation frequencies were applied, from 1 to 20 Hz, but for quantification only the first pulse of the train was used. The inter-train interval was typically >30 second long, to minimally affect synaptic depression or facilitation pathways. To test whether ChR2 responses are monosynaptic, TTX (1 μM) was applied to the bath until the EPSC response was eliminated, which was followed by application of 4 Amino-Pyridine (4AP, 100 μM). Typically, the response was recovered within several minutes, with a slightly longer delay between LED onset and EPSC generation. NBQX (10 μM) was applied to test if the responses were driven by AMPA receptors.

Slice preparation and patch clamp recordings of in vitro hCS. Acute hCS slices were generated as previously described³. Briefly, hCS slices were embedded in 4% agarose and transferred to an artificial cerebrospinal fluid (aCSF) containing 126 mM NaCl, 2.5 mM KCl, 1.25 mM NaH₂PO₄, 1 mM MgSO₄, 2 mM CaCl₂), 26 mM NaHCO₃ and 10 mM D-(+)-glucose. Slices were cut at 200-300 μm at room temperature using a Leica VT1200 vibratome and maintained in aCSF at room temperature. Whole-cell patch-clamp recordings from hCS slices were then performed under an upright SliceScope microscope (Scientifica). Slices were perfused with aCSF (bubbled with 95% O₂ and 5% CO₂), and signals from cells were recorded at room temperature. hCS neurons were patched with a borosilicate glass pipette filled with an internal solution containing 127 mM potassium gluconate, 8 mM NaCl, 4 mM magnesium ATP, 0.3 mM sodium GTP, 10 mM HEPES and 0.6 mM EGTA, pH 7.2, adjusted with KOH (290 mOsm). For reconstruction purposes, 0.2% biocytin was added to the internal solution.

Data were acquired with a MultiClamp 700B Amplifier (Molecular Devices) and a Digidata 1550B Digitizer (Molecular Devices), low pass filtered at 2 kHz, digitized at 20 kHz and analyzed using Clampfit (version 10.6, Molecular Devices) and custom Matlab (Mathworks) functions. The liquid junction potential was calculated using JPCalc, and recordings were corrected with an estimated-14 mV liquid junction potential. The I-V manipulations were constructed from a series of current steps in 5-10 pA increments from −50 to 250 pA.

Streptavidin staining and neuron tracing. For morphological reconstruction of patch-clamped neurons, 0.2% biocytin (Sigma-Aldrich) was added to the internal solution. Cells were filled for at least 15 min after break-in. Pipettes were then retracted slowly, over 1-2 minutes, until the recorded membrane fully resealed. Following slice physiology procedures, slices were post-fixed in 4% PFA overnight at 4° C. and then washed with PBS×3 before incubated with streptavidin-conjugated DyLight 549 (Vector Labs) at 1:1000 dilution for 2 h at room temperature to label cells that were filled with biocytin (2%, Sigma-Aldrich) during patch clamp recordings. Slices were then mounted for microscopy on glass slides using Aquamount (Thermo Scientific) and imaged the next day on Leica TCS SP8 confocal microscope, using a 40×1.3 NA, oil immersion objective, at 0.9×-1.0× zoom with an XY sampling frequency of ˜7 pixels/μm. Z stacks at 1 μm intervals were serially obtained and z-stack tiling and Leica-based automated stitching were performed to cover the entire dendritic tree of each neuron. The neurons were subsequently semi-manually traced using the neuTube interface (Feng, L. et al. eNeuro. 2015 Jan. 2; 2 (1): ENEURO.0049-14.2014) and SWC files were generated. The files were next loaded into Fiji (ImageJ, version 2.1.0, NIH) plugin Simple Neurite Tracer (Arshadi, C. et al. Nat Methods. 2021 April; 18 (4): 374-377).

Primary human sample collection. Human cerebral cortical tissue was obtained with informed consent under a protocol approved by the Stanford University Institutional Review Board (12625). The two postnatal human tissue samples (age 3- and 18-year-old) were obtained from resection of frontal lobe cortex (middle frontal gyrus) as part of surgeries for treating medically refractory epilepsy. After resection, tissue was collected in ice-cold NMDG-aCSF containing: 92 mM NMDG, 2.5 mM KCl, 1.25 mM NaH₂PO₄, 30 mM NaHCO₃, 20 mM HEPES, 25 mM glucose, 2 mM thiourea, 5 mM Na-ascorbate, 3 mM Na-pyruvate, 0.5 mM CaCl2·4H2O and 10 mM MgSO4·7H2O. The pH was titered to 7.3-7.4 with concentrated hydrochloric acid. The tissue was transferred to the lab within 30 minutes and coronal sections were made per the procedure described above.

Fiber implantation for fiber photometry. All animal procedures followed animal care guidelines approved by Stanford University's Administrative Panel on Laboratory Animal Care (APLAC). Rats (>140 days after transplantation) were anesthetized with 5% isoflurane for induction and 1-3% isoflurane during surgery. Animals were placed into a stereotactic frame (Kopf) and buprenorphine SR was administered subcutaneously. The skull was exposed, cleaned and 3-5 bone screws were inserted. To target the t-hCS we generated stereotactic coordinates based on the images acquired with MRI. A burr hole was drilled at the site of interest, and a fiber (400 μm diameter, 0.48 NA, Doric) was lowered to 100 μm below the surface of the hCS and affixed to the skull with UV-cured dental cement (Relyx).

Fiber photometry recordings. Fiber photometry recordings were performed as described previously³⁸. For recordings of spontaneous activity, rats were placed into a clean homecage and a 400 μm diameter fiber-optic patch cord (Doric) coupled to the fiber photometry acquisition system was connected to the implanted optical fiber. Animals were free to explore the homecage during a 10 min recording of spontaneous activity. For recordings of evoked activity, rats (>140 days after transplantation) were anesthetized with 5% isoflurane for induction and 1-3% isoflurane for maintenance. Animals were placed into a stereotactic frame (Kopf) and whiskers contralateral to the t-hCS were trimmed to ˜2 cm and threaded through mesh that was coupled to a piezo-electric actuator (PI). A 400 μm diameter fiber-optic patch cord (Doric) coupled to the acquisition system was connected to the implanted optical fiber. Fifty deflections (2 mm, 20 Hz, 2 s per presentation) were then made to the whiskers contralateral to the t-hCS using a piezo-electric actuator at random times during a 20 min recording. Deflection timing was controlled using custom Matlab code using the Matlab Support Package for Arduino. Events were synchronized with the acquisition software using TTL pulses.

Cranial window surgery. All animal procedures followed animal care guidelines approved by Stanford University's Administrative Panel on Laboratory Animal Care (APLAC). Rats (>140 days after transplantation) were anesthetized with 5% isoflurane for induction and 1-3% isoflurane during surgery. Animals were placed into a stereotactic frame (Kopf) and buprenorphine SR and dexamethasone were administered subcutaneously. The skull was exposed, cleaned and 3-5 bone screws were inserted. To target t-hCS, generated stereotactic coordinates were generated based on the images acquired with MRI. A circular craniotomy (˜1 cm diameter) was made with a high-speed drill directly above the transplanted hCS. Once the bone was as thin as possible, but before drilling all the way through the bone, the remaining intact bone disk was removed using forceps to reveal the underlying t-hCS. The craniotomy was filled with sterile saline and a glass coverslip and custom headbar were affixed to the skull with UV-cured dental cement (Relyx).

Acute in vivo two-photon calcium imaging. Two-photon imaging was performed using a Bruker multiphoton microscope using Nikon LWD objective (16×, 0.8 NA). Imaging of GCaMP6 was conducted at 920 nm, 1.4× zoom in a single z-plane, with 8× frame averaging at 7.5 frames/s. Rats were anesthetized with 5% isoflurane for induction and maintained with 1-3% isoflurane. Rats were placed into a custom head-fixed apparatus and positioned beneath the objective. A 3 min baseline recording of spontaneous activity was obtained. Fifty airpuffs (100 ms duration per presentation) were then delivered to the whisker pad contralateral to the t-hCS using a picospritzer at random times during a 20-min recording. Airpuff timing was controlled using custom Matlab code using the Matlab Support Package for Arduino. Events were synchronized with the acquisition software (PrairieView) using TTL pulses. For analysis, images were corrected for x-y motion using affine corrections in MoCo, running in Fiji. Fluorescence traces from individual cells were extracted using CNMF-E (Zhou, P. et al. Elife. 2018 Feb. 22; 7: e28728) Fluorescence from each ROI was extracted, converted to a dF/F trace, and then converted into a z-score.

Acute in vivo extracellular electrophysiology. Rats (>140 days after transplantation) were anesthetized with 5% isoflurane for induction and 1-3% isoflurane during surgery. Animals were placed into a stereotactic frame (Kopf) and buprenorphine SR and dexamethasone were administered subcutaneously. Whiskers contralateral to the t-hCS were trimmed to ˜2 cm and threaded through mesh that was coupled to a piezo-electric actuator (PI). The skull was exposed and cleaned. A stainless-steel ground screw was fastened to the skull. To target the t-hCS stereotactic coordinates were generated based on the images acquired with MRI. A circular craniotomy (˜1 cm diameter) was made with a high-speed drill directly above the t-hCS. Once the bone was as thin as possible, but before drilling all the way through the bone, the remaining intact bone disk was removed using forceps to reveal the underlying t-hCS. Single units were recorded using either 32 or 64 channel high-density silicon probes (Cambridge Neurotech) grounded to the ground screw and pre-amplified with an RHD amplifier (Intan). Electrodes were lowered through the craniotomy into the target site using a manipulator and the craniotomy was filled with sterile saline. Data acquisition was performed at 30 KHz with an Open Ephys acquisition system. Recordings only proceeded if more than 10 channels were identified with highly correlated, rhythmic spontaneous activity suggesting that electrodes were positioned in the graft (based on two-photon calcium imaging data). A 10-min baseline recording of spontaneous activity was obtained. Fifty deflections (2 mm, 20 Hz, 2 s per presentation) were then made to the whiskers contralateral to the t-hCS using a piezo-electric actuator at random times during a 20-min recording. Deflection timing was controlled using custom Matlab code using the Matlab Support Package for Arduino. Events were synchronized with the acquisition software using TTL pulses.

For opto-tagging experiments a 200 μm diameter fiber-optic patch cord (Doric), coupled to a 473 nm laser (Omicron) was connected to a 200 μm diameter optical fiber that was positioned above the craniotomy. Immediately before this, the power output from the patch cord was adjusted to 20 mW. Electrodes were lowered through the craniotomy into the target site using a manipulator and the craniotomy was filled with sterile saline. 10 pulses of 473 nm light (2 Hz, 10 ms pulse width) were delivered at the start of the recording. Light-responsive units were defined as units that displayed a spiking response within 10 ms of light onset on ≥70% of trials.

For analysis, spikes were sorted using Kilosort2 and were manually curated using Phy2 (Stringer, C. et al. Nature. 2019 July; 571 (7765): 361-365). Firing rates were computed using 200 ms bins, with a sliding window of 100 ms and converted into a z-score. A hidden Markov model with two states was used to label ‘On’ and ‘Off’ states in the population activity. ‘On’ states were considered to represent bursts, and ‘Off’ states were considered to represent inter-burst intervals. The emission transition parameters of the model were fit using the Baum-Welch algorithm (Matlab hmmtrain with a convergence threshold of 1×10⁻⁶ and initial guesses of transition matrix: [0.95, 0.05; 0.05, 0.96] and emission: [0.5, 0.5; 0.1, 0.99]), and the state assignment at each time point was then estimated using the Viterbi algorithm. To assess responses to whisker deflection, a Wilcoxon signed rank test was performed to compare firing rates in the 1 s following the onset of whisker deflection to the 1 s prior to whisker deflection with a significance threshold of P<0.05. Latencies were computed as the time to reach peak z-score in the 2 s following whisker deflection. The power spectral density was calculated using Welch's method (pwelch( ) in Matlab), with a window side of 10×fs, where fs is the sampling rate of the signal.

Fiber implantation for optogenetic manipulations. Rats (>90 days after transplantation) were anesthetized with 5% isoflurane for induction and 1-3% isoflurane during surgery. Animals were placed into a stereotactic frame (Kopf) and buprenorphine SR was administered subcutaneously. The skull was exposed, cleaned and 3-5 bone screws were inserted. To target the t-hCS stereotactic coordinates were generated based on the images acquired with MRI. A burr hole was drilled at the site of interest, and a fiber (200 μm diameter, 0.48 NA, Thorlabs) was lowered to 100 μm below the surface of the hCS and affixed to the skull with UV-cured dental cement (Relyx).

Optogenetic behavioral assay. Water scheduled rats (1 h water per day) were placed into a custom operant chamber containing a nosepoke portal equipped with a lick spout for water reward delivery. Entries into the nosepoke portal were detected by the breakage of an infra-red beam and licks were detected using a capacitive touch sensor. All events were controlled and recorded using custom Matlab code using the Matlab Support Package for Arduino. At least one week after surgery animals began pre-training. On day one of pre-training animals received small water rewards at the reward spout at randomized delays for 1 h. On days two and three of pre-training animals received small water rewards only after performing increasing numbers of licks to the lick spouts for 1 h hour. All animals readily performed this behavior. After pre-training animals were trained to associate optogenetic stimulation of the transplanted hCS with reward delivery. Animals were placed into the operant chamber and a 200 μm diameter fiber-optic patch cord (Doric), coupled to both a 473 nm (Omicron) and 635 nm (CNI) laser outside of the operant chamber was connected to the implanted optical fiber. Immediately before this, the power output from the patch cord was adjusted to 20 mW. Laser timing was controlled by a Master-8 pulse generator (AMPI). 1 s after entering the nosepoke portal, animals received random presentations of either 473 nm or 635 nm stimulation (10 Hz, 10 ms pulse width, 5 s total stimulation). If animals performed one or more licks during 473 nm stimulation a small water reward was dispensed at the reward spout after stimulation was complete. The next trial was initiated after collection of this reward. If animals performed one or more licks during 635 nm stimulation, there was no consequence. Trials were separated by a variable interval of 5-10 s. Animals received daily training sessions that concluded after 150 473 nm and 150 635 nm trials had been completed or after 150 min, whichever came first, for a total of 15 days. Behavioral performance was quantified by calculating a preference index for each training session: (#licks during 473 nm stimulation—#licks during 635 nm stimulation)/(#licks during 473 nm stimulation+ #licks during 635 nm stimulation).

Optogenetic stimulation and c-Fos staining. Rats were placed into a clean rat cage and a 200 μm diameter fiber-optic patch cord (Doric), coupled to a 473 nm (Omicron) laser was connected to the implanted optical fiber. Immediately before this, the power output from the patch cord was adjusted to 20 mW. Stimulated animals received 10 Hz, 10 ms pulse width, 473 nm stimulation for 10 minutes before being returned to their homecage. Unstimulated animals received no stimulation for 10 minutes before being returned to their homecage. Rats were euthanized by transcardial perfusion with 150 ml PBS, followed by 100 ml 4% paraformaldehyde 90 minutes after optogenetic stimulation. Brains were extracted and 100 μm sections were cut on a vibratome. The slices were labelled with goat anti-GFP (Abcam) and rabbit anti-c-Fos (Abcam) primary antibodies, Alexa 488 donkey anti-goat (Invitrogen) and Alexa 594 donkey anti-rabbit (Invitrogen) secondary antibodies, and DAPI. Images were acquired using a confocal microscope (Zeiss) with a 20× objective and overlaid with images from the Paxinos, George and Watson rat brain atlas for blinded manual counting of c-Fos positive cells in specified brain regions.

EEG recordings. FOXN1^{-/-} rats were anesthetized with isoflurane and stereotactically implanted with stainless steel wires (A-M Systems, 791400) soldered to screws (J.I. Morris, FFOOCE125) over the bilateral somatosensory cortices, the bilateral motor cortices. A reference wire was positioned over the cerebellum, and implants were secured with dental cement (Metabond, S399, S371 and S398; also Jet Set4 Liquid, Lang Dental, 3802×6). The following stereotactic coordinates were used, relative to bregma: primary somatosensory cortex (S1BF), AP-1.3 mm and lateral 3.3 mm; primary motor cortex (M1), AP+2.5 mm, lateral 2.5 mm. The wires of the implant were secured onto custom-made Mill-Max headpiece adapters (Digi-Key Electronics, ED90267-ND). To initiate the recording the adapters were connected to the head stage, consisting of a digitizer and amplifier board (Intan Technologies, C3334). Awake, freely behaving animals were tethered to an acquisition board (Open Ephys) with lightweight SPI interface cables (Intan Technologies, C3206). Continuous real-time EEG was recorded with Open Ephys software (https://open-ephys.org, version 0.4.4.1). Data were sampled at 2 kHz and bandpass filtered between 1 Hz and 300 Hz.

Assessment of locomotor behavior in open field arena. Rats (>3 months old, >90 days post-transplantation) were handled for 3 min on 5 consecutive days before behavioral testing began. Rats were placed into the corner of an Open Field Activity Arena (43 cm×43 cm×30 cm) containing 3 planes of infrared detectors within a sound attenuating chamber (Med Associates). Rats were allowed to explore the arena for 10 min and distance moved was computed with Med Associates software. The arena was cleaned with a 1% Virkon solution at the end of each session.

Novel object recognition. Two different objects (green tower and white bottle) were used in this test. The objects had similar heights and volumes, but differed in their shape, texture, and appearance. On day 1, rats were placed into a black square plastic area (50 cm×50 cm×45 cm) and allowed to explore the arena with 2 habituation objects (15 ml centrifuge tubes) for 5 min. On day 2, rats first underwent a training session. In this session, rats were placed into the arena and allowed to explore the arena for 5 min with 2 identical objects which were placed in diagonally opposite corners 15 cm away from the corner. Rats were then returned to their homecage for 5 min. For the testing session, rats were placed back into the arena for 5 min and one of the two objects was replaced with a novel object. Rats were tracked with an automated tracking system (Noldus Information Technology) and time spent interacting with each object was manually scored by an experimenter who was blind to the experimental groups. Interaction was defined as the rat pointing its nose towards the object within 2 cm of it. Objects for training and testing and the location of these objects were pseudorandomized. Objects and the arena were cleaned with a 1% Virkon solution at the end of each training and testing session. A discrimination index was calculated as (time spent interacting with novel object-time spent interacting with familiar object)/(time spent interacting with novel object+time spent interacting with familiar object) during the testing session.

Fear conditioning. The experiment consisted of one day of training, one day of contextual fear testing, and one day of cued fear testing. The same context was used for both training and contextual testing. This context had aluminum walls, a gray metal grid for a floor, yellow house lights and the scent of mint extract, and was cleaned with 10% simple green solution between rats (Coulbourn Instruments). The cued fear testing context was circular with plastic walls and floor, blue house lights and the scent of vanilla extract, and was cleaned with 70% ethanol between rats. On day 1 (training), rats were placed individually into the training chamber for 200 seconds. A tone (20 s, 80 dB, 2 KHz) was presented followed by an electrical shock (0.5 mA, 2 s duration) 18 s after the end of the tone. This procedure was repeated a total of 3 times with 60 second intervals between the end of the shock and the start of the subsequent tone. Rats were removed from the chamber and returned to their home cage 60 seconds after the last shock. On day 2, rats were placed back in the training context without any tone or shock for 5 minutes for contextual memory testing. On day 3, the rats were placed in the cued fear context. After 200 seconds of habituation, rats were presented with tones (20 s, 80 dB, 2 KHz) 3 times at 80 second intervals. Stimulus presentation was controlled using FreezeFrame software. An overhead camera was used to record behavior. Freezing behavior was scored manually by an experimenter who was blind to the experimental groups. Freezing was defined as the absence of all movements except those caused by respiration.

Statistics. Data are presented as meants.e.m., unless otherwise indicated. Distribution of the raw data was tested for normality of distribution; statistical analyses were performed using the Student's t-test, Kolmogorov Smirnov test, Wilcoxon signed-rank test or ANOVA with Bonferroni correction for multiple comparisons. Sample sizes were estimated empirically or based on power calculations.

### REFERENCES

1. Kelley, K. W. & Pasca, S. P. Human brain organogenesis: Toward a cellular understanding of development and disease. Cell (2021) doi: https://doi.org/10.1016/j.cell.2021.10.003.
2. Pasca, A. M. et al. Functional cortical neurons and astrocytes from human pluripotent stem cells in 3D culture. Nature Methods 12, (2015).
3. Valesco, S. et al. Individual brain organoids reproducibly form cell diversity of the human cerebral cortex. Nature 570, (2019).
4. Qian, X. et al. Brain-region-specific organoids using mini-bioreactors for modeling ZIKV exposure. Cell 165, (2016).
5. Yoon, S. J. et al. Reliability of human cortical organoid generation. Nature Methods 16, (2019).
6. Birey, F. et al. Assembly of functionally integrated human forebrain spheroids. Nature 545, (2017).
7. Espuny-Camacho, I. et al. Pyramidal neurons derived from human pluripotent stem cells integrate efficiently into mouse brain circuits in vivo. Neuron 77, (2013).
8. Linaro, D. et al. Xenotransplanted Human Cortical Neurons Reveal Species-Specific Development and Functional Integration into Mouse Visual Circuits. Neuron 104, (2019).
9. Mansour, A. A. et al. An in vivo model of functional and vascularized human brain organoids. Nature Biotechnology 36, (2018).
10. Real, R. et al. In vivo modeling of human neuron dynamics and down syndrome. Science (2018) doi: 10.1126/science.aau1810.
11. Kitahara, T. et al. Axonal Extensions along Corticospinal Tracts from Transplanted Human Cerebral Organoids. Stem Cell Reports 15, (2020).
12. Xiong, M. et al. Human Stem Cell-Derived Neurons Repair Circuits and Restore Neural Function. Cell Stem Cell 28, (2021).
13. Ebert, D. H. & Greenberg, M. E. Activity-dependent neuronal signalling and autism spectrum disorder. Nature vol. 493 (2013).
14. Kichula, E. A. & Huntley, G. W. Developmental and comparative aspects of posterior medial thalamocortical innervation of the barrel cortex in mice and rats. Journal of Comparative Neurology 509, (2008).
15. Hrvatin, S. et al. Single-cell analysis of experience-dependent transcriptomic states in the mouse visual cortex. Nature Neuroscience 21, (2018).
16. Ataman, B. et al. Evolution of Osteocrin as an activity-regulated factor in the primate brain. Nature 539, (2016).
17. Hong, E. J., McCord, A. E. & Greenberg, M. E. A Biological Function for the Neuronal Activity-Dependent Component of Bdnf Transcription in the Development of Cortical Inhibition. Neuron 60, (2008).
18. Polioudakis, D. et al. A single-cell transcriptomic atlas of human neocortical development during mid-gestation. Neuron 103, (2019).
19. Trevino, A. E. et al. Chromatin and gene-regulatory dynamics of the developing human cerebral cortex at single-cell resolution. Cell 184, (2021).
20. Hodge, R. D. et al. Conserved cell types with divergent features in human versus mouse cortex. Nature 573, (2019).
21. Bakken, T. E. et al. Comparative cellular analysis of motor cortex in human, marmoset and mouse. Nature 598, (2021).
22. Zhu, Y. et al. Spatiotemporal transcriptomic divergence across human and macaque brain development. Science 362, (2018).
23. Gordon, A. et al. Long-term maturation of human cortical organoids matches key early postnatal transitions. Nature Neuroscience 24, (2021).
24. Krey, J. F. et al. Timothy syndrome is associated with activity-dependent dendritic retraction in rodent and human neurons. Nature Neuroscience 16, (2013).
25. Agmon, A. & Connors, B. W. Thalamocortical responses of mouse somatosensory (barrel) cortex in vitro. Neuroscience 41, (1991).
26. Petreanu, L., Mao, T., Sternson, S. M. & Svoboda, K. The subcellular organization of neocortical excitatory connections. Nature 457, (2009).
27. Kalmbach, B. E. et al. h-Channels Contribute to Divergent Intrinsic Membrane Properties of Supragranular Pyramidal Neurons in Human versus Mouse Cerebral Cortex. Neuron 100, (2018).
28. Molnar, Z., Luhmann, H. J. & Kanold, P. O. Transient cortical circuits match spontaneous and sensory-driven activity during development. Science 370, (2020).
29. Matson, K. J. E. et al. Isolation of adult spinal cord nuclei for massively parallel single-nucleus RNA sequencing. Journal of Visualized Experiments 2018, (2018).
30. Stuart, T. et al. Comprehensive Integration of Single-Cell Data. Cell 177, (2019).
31. McGinnis, C. S., Murrow, L. M. & Gartner, Z. J. DoubletFinder: Doublet Detection in Single-Cell RNA Sequencing Data Using Artificial Nearest Neighbors. Cell Systems 8, (2019).
32. Becht, E. et al. Dimensionality reduction for visualizing single-cell data using UMAP. Nature Biotechnology 37, (2019).
33. Nowakowski, T. J. et al. Spatiotemporal gene expression trajectories reveal developmental hierarchies of the human cortex. Science 358, (2017).
34. Chen, J., Bardes, E. E., Aronow, B. J. & Jegga, A. G. ToppGene Suite for gene list enrichment analysis and candidate gene prioritization. Nucleic Acids Research 37, (2009).
35. Huguenard, J. R. & Prince, D. A. Intrathalamic rhythmicity studied in vitro: Nominal T-current modulation causes robust antioscillatory effects. Journal of Neuroscience 14, (1994).
36. Feng, L., Zhao, T. & Kim, J. Neutube 1.0: A new design for efficient neuron reconstruction software based on the swc format. eNeuro 2, (2015).
37. Arshadi, C., Günther, U., Eddison, M., Harrington, K. I. S. & Ferreira, T. A. SNT: a unifying toolbox for quantification of neuronal anatomy. Nature Methods 18, (2021).
38. Steinberg, E. E., et al. Amygdala-Midbrain Connections Modulate Appetitive and Aversive Learning. Neuron 106, (2020).
39. Zhou, P. et al. Efficient and accurate extraction of in vivo calcium signals from microendoscopic video data. eLife 7, (2018).
40. Stringer, C., Pachitariu, M., Steinmetz, N., Carandini, M. & Harris, K. D. High-dimensional geometry of population responses in visual cortex. Nature 571, (2019).

Notwithstanding the appended claims, the disclosure is also defined by the following clauses:

1. A method of producing a non-human mammalian animal model comprising human neural tissue, the method comprising:

introducing a first human neural organoid into a central nervous system location of a newborn non-human mammal; and

allowing the newborn non-human mammal to mature to produce the non-human mammalian animal model comprising human neural tissue.

2 The method of clause 1, wherein the newborn non-human mammal is a rodent.

3. The method of clause 2, wherein the rodent is a rat.
4. The method of clause 2, wherein the rodent is a mouse.
5. The method of clause 1, wherein the newborn non-human mammal is a primate.
6. The method of any of the preceding clauses, wherein the newborn non-human mammal is an immunocompromised non-human mammal.
7. The method of clause 6, wherein the immunocompromised non-human mammal comprises a genetic mutation.
8. The method of clause 6, wherein the immunocompromised non-human mammal is immunocompromised as result of a chemical treatment.
9. The method of any of the preceding clauses, wherein the first human neural organoid is an organoid generated from induced human pluripotent stem cells (hiPSCs).
10. The method of any of the preceding clauses, wherein the first neural organoid comprises a striatal organoid.
11. The method of clause 10, wherein the striatal organoid comprises GABAergic medium spiny neurons that develop dendritic spines.
12. The method of any of the preceding clauses, wherein the first neural organoid comprises a ventral forebrain organoid.
13. The method of any of the preceding clauses, wherein the first neural organoid comprises a cortical organoid.
14. The method of clause 12 or 13, wherein the neural organoid comprises functional glutamatergic neurons.
15. The method of any of clauses 1-11, wherein the first neural organoid comprises a midbrain organoid.
16. The method of clause 15, wherein the midbrain organoid comprises dopaminergic neurons, including those resembling neurons in the substantia nigra.
17. The method of any of the preceding clauses, wherein the first neural organoid is derived from a human having a neuropsychiatric disorder.
18. The method of clause 17, wherein the neuropsychiatric disorder is selected from the group consisting of Timothy syndrome, tuberous sclerosis, 22q11.2 deletion syndrome, autism spectrum disorder, epilepsy, schizophrenia Huntington's disease, Parkinson's disease, and Tourette's syndrome.
19. The method of any of the preceding clauses, wherein the central nervous system location is selected from the group consisting of the frontal cortex, the motor cortex, somatosensory cortex, parietal cortex, occipital cortex, temporal cortex, striatum, spinal cord, thalamus, and the cerebellum.
20. The method of any of the preceding clauses, wherein the newborn non-human mammal is from 1 to 7 days old.
21. The method of any of the preceding clauses, further comprising introducing a second human neural organoid to a second central nervous system location of the newborn non-human mammal.
22. The method of clause 21, wherein the second human neural organoid is the same as the first human neural organoid.
23. The method of clause 21, wherein the second human neural organoid is different from the first human neural organoid.
24. The method of any of clauses 21-23, wherein the second human neural organoid is introduced to the same central nervous system location to which the first human neural organoid is introduced.
25. The method of any of clauses 21-23, wherein the second human neural organoid is introduced to the same central nervous system location to which the first human neural organoid is introduced but in the opposite brain hemisphere.
26. The method of any of clauses 21-23, wherein the second human neural organoid is introduced to a second central nervous system location that is different from the central nervous system location to which the first human neural organoid is introduced.
27. The method of any of the preceding clauses, wherein the non-human mammalian animal model comprises anatomically integrated human neural tissue with advanced features of maturation (morphological and functional).
28. The method of any of the preceding clauses, wherein the non-human mammalian animal model comprising anatomically integrated human neural tissue receives physiological sensory input from said human neural tissue.
29. The method of any of the preceding clauses, wherein the non-human mammalian animal model comprising anatomically integrated human neural tissue is vascularized.
30. A method of modeling a neuropsychiatric disorder, the method comprising:
introducing a first human neural organoid produced from a cellular biological sample from an individual living with a neuropsychiatric disorder into a first central nervous system location of a newborn non-human mammal;
allowing the newborn non-human mammal to mature to produce the non-human mammalian animal model comprising a first human neural tissue; and
characterizing the first human neural tissue to model the neuropsychiatric disorder.
   31. The method of clause 30, wherein the newborn non-human mammal is a rodent.
   32. The method of clause 30, wherein the rodent is a rat.
   33. The method of clause 30, wherein the rodent is a mouse.
   34. The method of clause 20, wherein the non-human mammal is a primate.
   35. The method of any of clauses 30-34, wherein the non-human mammal is an immunocompromised non-human mammal.
   36. The method of clause 35, wherein the immunocompromised non-human mammal comprises a genetic mutation.
   37. The method of clause 35, wherein the immunocompromised non-human mammal is immunocompromised as result of a chemical treatment.
   38. The method of any of clauses 30-37, wherein the first neural organoid comprises a striatal organoid.
   39. The method of clause 38, wherein the striatal organoid comprises GABAergic medium spiny neurons that develop dendritic spines.
   40. The method of any of clauses 30-39, wherein the first neural organoid comprises a ventral forebrain organoid.
   41. The method of any of clauses 30-40, wherein the first neural organoid comprises a cortical organoid.
   42. The method of clause 41, wherein the cortical organoid comprises functional glutamatergic neurons.
   43. The method of any of clauses 30-39, wherein the first neural organoid comprises a midbrain organoid.
   44. The method of clause 43, wherein the midbrain organoid comprises dopaminergic neurons, including substantia nigra.
   45. The method of any of clauses 30-44, wherein the neuropsychiatric disorder is selected from the group of Timothy syndrome, tuberous sclerosis, 22q11.2 deletion syndrome, Autism spectrum disorder, Epilepsy, Schizophrenia Huntington's disease, Parkinson's disease, and Tourette's syndrome.
   46. The method of any of clauses 30-45, wherein the central nervous system location is selected from the group consisting of the frontal cortex, motor cortex, somatosensory cortex, parietal cortex, occipital cortex, temporal cortex, striatum, spinal cord, thalamus, and the cerebellum.
   47. The method of any of clauses 30-46, wherein the newborn non-human mammal is from 1 to 7 days old.
   48. The method of any of clauses 30-47, wherein the characterizing comprises measuring neuronal morphology of the human neural tissue.
   49. The method of any of clauses 30-48, wherein the characterizing comprises measuring intrinsic electrophysiological properties of the first human neural tissue.
   50. The method of any of clauses 30-49, wherein the characterizing comprises measuring maximal firing rates of the first human neural tissue.
   51. The method of any of clauses 30-50, wherein the characterizing comprises measuring gene expression in the first human neural tissue.
   52. The method of any of clauses 30-51, wherein the characterizing comprises immunostaining the first human neural tissue.
   53. The method of any of clauses 30-52, wherein the characterizing comprises axon tracing of the first human neural tissue.
   54. The method of any of clauses 30-53, wherein the characterizing comprises measuring intracellular calcium levels or voltage.
   55. The method of clause 54, wherein the human neural tissue comprises a calcium sensor.
   56. The method of clause 55, wherein the calcium sensor is GCaMP6 s.
   57. The method of any of clauses 30-56, further comprising characterizing the non-human mammalian animal model.
   58. The method of clause 57, wherein the characterizing comprises assaying the non-human mammalian animal models behavioral responses to tasks.
   59. The method of clause 57 or 58, wherein the characterizing comprises assaying the non-human mammalian animal models memory responses to tasks.
   60. The method of any of clauses 57-59, wherein the characterizing comprises assaying the non-human mammalian animal models motor responses to tasks.
   61. The method of any of clauses 57-60, wherein the characterizing comprises assaying the non-human mammalian animal models sensory responses to tasks.
   62. The method of any of clauses 30-61, further comprising introducing a second human neural organoid to a second central nervous system location of the newborn non-human mammal and characterizing a second human neural tissue that is produced from the second human neural organoid; wherein the second human neural organoid is derived from an individual that does not have a neuropsychiatric disorder.
   63. The method of clause 62, wherein the second human neural organoid is the same as the first human neural organoid except that it is derived from an individual that does not have a neuropsychiatric disorder.
   64. The method of clause 62, wherein the second human neural organoid is different from the first human neural organoid.
   65. The method of clause 62, wherein the second human neural organoid is introduce to the same central nervous system location to which the first human neural organoid is introduced.
   66. The method of clause 62, wherein the second human neural organoid is introduced to the same central nervous system location to which the first human neural organoid is introduced except in the opposite brain hemisphere.
   67. The method of any of clauses 62-66, wherein the second human neural organoid is introduced to a second central nervous system location that is different from the central nervous system location to which the first human neural organoid is introduced.
   68. The method of any of clauses 30-67, wherein the producing comprises:
obtaining the cellular biological sample from the individual living with a neuropsychiatric disorder;
converting cells of the cellular biological sample into induced pluripotent stem cells; and
differentiating the induced pluripotent stem cells into the first human neural organoid.
   69. A method of determining the effectiveness of a candidate agent on a neuropsychiatric disorder, the method comprising:
administering the drug to the non-human mammalian animal model produced by any of the methods of clauses 30-68;
assaying the first human neural tissue; and
comparing the results of the assaying with mammals administered a control agent that is not the candidate agent.
   70. The method of clause 69, wherein the neuropsychiatric disorder is selected from the group consisting of Timothy syndrome, tuberous sclerosis, 22q11.2 deletion syndrome, Autism spectrum disorder, Epilepsy, Schizophrenia, Huntington's disease, Parkinson's disease, and Tourette's syndrome . . .
   71. The method of clause 69 or 70, wherein the candidate agent is selected from the group consisting of a chemical, a small molecule, a gene therapy, and an antibody.
   72. The method of any of clauses 69-71, wherein the candidate agent is administered systemically.
   73. The method of any of clauses 69-71, wherein the candidate agent is administered locally at the site of the human neural tissue.
   74. The method of any of clauses 69-73, wherein the assaying comprises measuring neuronal morphology of the human neural tissue.
   75. The method of any of clauses 69-74, wherein the assaying comprises measuring intrinsic electrophysiological properties of the human neural tissue.
   76. The method of any of clauses 69-75, wherein the assaying comprises measuring maximal firing rates of the human neural tissue.
   77. The method of any of clauses 69-76, wherein the assaying comprises measuring gene expression in the human neural tissue.
   78. The method of any of clauses 69-77, wherein the assaying comprises immunostaining the human neural tissue.
   79. The method of any of clauses 69-78, wherein the assaying comprises axon tracing of the human neural tissue.
   80. The method of any of clauses 69-79, wherein the characterizing comprises measuring intracellular calcium levels.
   81. The method of clause 80, wherein the human neural tissue comprises a calcium sensor.
   82. The method of clause 81, wherein the calcium sensor is GCaMP6 s.
   83. The method of any of clauses 69-82, wherein the mammal comprises a second human neural organoid introduced to a central nervous system location of the newborn non-human mammal.
   84. The method of clause 83, wherein the second neural organoid is different from the first neural organoid.
   85. The method of clause 83 or 84, wherein the second neural organoid is in the same central nervous system location as the first neural organoid.
   86. The method of clause 83 or 84, wherein the second human neural organoid is introduced to the same central nervous system location to which the first human neural organoid is introduced except in the opposite brain hemisphere.
   87. The method of clause 83 or 84, wherein the second neural organoid is in a different central nervous system location as the first neural organoid.
   88. The method of any of clauses 69-87, wherein the producing comprises:
obtaining the cellular biological sample from the individual living with a neuropsychiatric disorder;
converting cells of the cellular biological sample into induced pluripotent stem cells; and
differentiating the induced pluripotent stem cells into the first human neural organoid.
   89. A method for altering the behavior of a mammal, the method comprising:
introducing a first human neural organoid into a first central nervous system location of a newborn non-human mammal;
allowing the newborn non-human mammal to mature to produce a non-human mammal comprising a first human neural tissue;
implanting an optical fiber into the first human neural tissue;
training the mammal on an operant conditioning paradigm using the optical fiber; and
stimulating the first human neural tissue using the optical fiber, wherein the first human neural tissue comprises a first light activatable polypeptide, wherein stimulating the first human neural tissue results in a change in the activity of the neurons in said first human neural tissue;
to modify the behavior of the mammal and obtain a behavioral readout of the human graft.
   90. The method of clause 89, wherein the newborn non-human mammal is a rodent.
   91. The method of clause 90, wherein the rodent is a rat.
   92. The method of clause 90, wherein the rodent is a mouse.
   93. The method of clause 89, wherein the newborn non-human mammal is a primate.
   94. The method of any of clauses 89-93, wherein the newborn non-human mammal is an immunocompromised non-human mammal.
   95. The method of clause 94, wherein the immunocompromised non-human mammal comprises a genetic mutation.
   96. The method of clause 94, wherein the immunocompromised non-human mammal is immunocompromised as result of a chemical treatment.
   97. The method of any of clauses 89-96, wherein the first human neural organoid is an organoid generated from induced human pluripotent stem cells (hiPSCs).
   98. The method of any of clauses 89-97, wherein the first neural organoid comprises a striatal organoid.
   99. The method of clause 98, wherein the striatal organoid comprises GABAergic medium spiny neurons that develop dendritic spines.
   100. The method of any of clauses 89-99, wherein the first neural organoid comprises a ventral forebrain organoid.
   101. The method of any of clauses 89-100, wherein the first neural organoid comprises a cortical organoid.
   102. The method of clause 101, wherein the cortical organoid comprises functional glutamatergic neurons.
   103. The method of any of clauses 89-102, wherein the first neural organoid comprises a midbrain organoid.
   104. The method of clause 103, wherein the midbrain organoid comprises dopaminergic neurons, including substantia nigra.
   105. The method of any of clauses 89-104, wherein the first neural organoid is derived from a human having a neuropsychiatric disorder.
   106. The method of clause 105, wherein the neuropsychiatric disorder is selected from the group consisting of Timothy syndrome, tuberous sclerosis, 22q11.2 deletion syndrome, Autism spectrum disorder, Epilepsy, Schizophrenia, Huntington's disease, Parkinson's disease, and Tourette's syndrome.
   107. The method of any of clauses 89-106, wherein the first central nervous system location is selected from the group consisting of the frontal cortex, motor cortex, somatosensory cortex, parietal cortex, occipital cortex, temporal cortex, striatum, spinal cord, thalamus, and the cerebellum.
   108. The method of any of clauses 89-107, wherein the newborn non-human mammal is from 1 to 7 days old.
   109. The method of any of clauses 89-107, wherein the operant conditioning paradigm comprises positive reinforcement.
   110. The method of any of clauses 89-108, wherein the operant conditioning paradigm comprises negative reinforcement.
   111. The method of any of clauses 89-110, wherein the first light activable polypeptide hyperpolarizes the first human neural tissue.
   112. The method of any of clauses 89-110, wherein the first light activable polypeptide depolarizes the first human neural tissue.
   113. The method of clause 112, wherein the first light activatable polypeptide is a channel rhodopsin.
   114. The method of clause 113, wherein the channel rhodopsin is channelrhodopsin-2.
   115. The method of any of clauses 89-114, wherein the stimulating comprises exposing the first human neural tissue with a first and second light wavelength.
   116. The method of clause 115, wherein the first light wavelength is between 380 nm and 740 nm and the second light wavelength is between 380 nm and 740 nm.
   117. The method of any of clauses 89-116, wherein the altering increases the behavior of the mammal.
   118. The method of any of clauses 89-117, wherein the altering decrease the behavior of the animal.
   119. The method of any of clauses 89-118, further comprising introducing a second human neural organoid to a second central nervous system location of the newborn non-human mammal.
   120. The method of clause 119, wherein the second cerebral organoid is the same as the first neural organoid.
   121. The method of clause 119, wherein the second neural organoid is different from the first cerebral organoid.
   122. The method of any of clauses 119-121, wherein the second neural organoid is introduced in the same region of the central nervous system as the first neural organoid.
   123. The method of any of clauses 119-121, wherein the second human neural organoid is introduced to the same central nervous system location to which the first human neural organoid is introduced except in the opposite brain hemisphere.
   124. The method of any of clauses 119-121, wherein the second neural organoid is introduced in a different region of the central nervous system as the first neural organoid.
   125. The method of any of clauses 119-121, wherein the second neural organoid comprises a second light activatable channel.
   126. The method of any of clauses 119-125, wherein the second light activable polypeptide hyperpolarizes the first human neural tissue.
   127. The method of any of clauses 119-125, wherein the second light activable polypeptide depolarizes the first human neural tissue.
   128. The method of clause 127, wherein the second light activatable polypeptide is a channel rhodopsin.
   129. The method of clause 127 or 128, wherein the channel rhodopsin is channelrhodopsin-2.
   130. The non-human mammal produced by the method of any of clauses 1-129.
   131. A non-human mammal comprising anatomically integrated human neural tissue.
   132. The method of clause 131, wherein the newborn non-human mammal is a rodent.
   133. The method of clause 132, wherein the rodent is a rat.
   134. The method of clause 132, wherein the rodent is a mouse.
   135. The method of clause 131, wherein the newborn non-human mammal is a primate.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1. A method of producing a non-human mammalian animal model comprising human neural tissue, the method comprising:
introducing a first human neural organoid into a central nervous system location of a newborn non-human mammal; and
allowing the newborn non-human mammal to mature to produce the non-human mammalian animal model comprising human neural tissue.

2. The method of claim 1, wherein the newborn non-human mammal is a rodent, preferably a rat or mouse, or a primate.

3. The method of claim 1, wherein the newborn non-human mammal is an immunocompromised non-human mammal.

4. The method of claim 1, wherein the first human neural organoid is an organoid generated from induced human pluripotent stem cells (hiPSCs).

5. The method of claim 1, wherein the first neural organoid is derived from a human having a neuropsychiatric disorder.

6. The method of claim 1, wherein the central nervous system location is selected from the group consisting of the frontal cortex, the motor cortex, somatosensory cortex, parietal cortex, occipital cortex, temporal cortex, striatum, spinal cord, thalamus, and the cerebellum.

7. The method of claim 1, further comprising introducing a second human neural organoid to a second central nervous system location of the newborn non-human mammal.

8. The method of claim 1, wherein the non-human mammalian animal model comprises anatomically integrated human neural tissue with advanced features of maturation (morphological and functional).

9. The method of claim 1, wherein the non-human mammalian animal model comprising anatomically integrated human neural tissue receives physiological sensory input from said human neural tissue.

10. The method of claim 1, wherein the non-human mammalian animal model comprising anatomically integrated human neural tissue is vascularized.

11. A method of modeling a neuropsychiatric disorder, the method comprising:
introducing a first human neural organoid produced from a cellular biological sample from an individual living with a neuropsychiatric disorder into a first central nervous system location of a newborn non-human mammal;
allowing the newborn non-human mammal to mature to produce the non-human mammalian animal model comprising a first human neural tissue; and
characterizing the first human neural tissue to model the neuropsychiatric disorder.

12. A method of determining the effectiveness of a candidate agent on a neuropsychiatric disorder, the method comprising:
administering the drug to the non-human mammalian animal model produced by the method of claim 11;
assaying the first human neural tissue; and
comparing the results of the assaying with mammals administered a control agent that is not the candidate agent.

13. (canceled)

14. (canceled)

15. A non-human mammal comprising anatomically integrated human neural tissue.

16. The non-human mammal of claim 15, wherein the non-human mammal is a rodent.

17. The non-human mammal of claim 16, wherein the rodent is a rat.

18. The non-human mammal of claim 16, wherein the rodent is a mouse.

19. The non-human mammal of claim 16, wherein the non-human mammal is a primate.

20. The non-human mammal of claim 15, wherein the non-human mammal is a newborn.
